(19) United States
(12) Patent Application Publication
(10) Pub. No.: US 2008/0039616 A1
(43) Pub. Date: Feb. 14, 2008

(54) TANDEM AFFINITY PURIFICATION SYSTEMS AND METHODS UTILIZING SUCH SYSTEMS

(75) Inventors: Nathan Zenser, Rock Hill, MO (US); Kristin Bettinger, St. Louis, MO (US); Keming Song, Ballwin, MO (US)

Correspondence Address: SIGMA-ALDRICH CO.;LAW DEPARTMENT, 3050 SPRUCE STREET, ST. LOUIS, MO, 63013, US

(73) Assignee: Sigma-Aldrich Co., St. Louis, MO (US)

(21) Appl. No.: 11/537,064

(22) Filed: Sep. 29, 2006

Related U.S. Application Data
(60) Provisional application No. 60/721,781, filed on Sep. 29, 2005.
(60) Provisional application No. 60/722,786, filed on Sep. 30, 2005.

(51) Int. Cl. C12N 15/11; C07H 21/00; C12N 15/63; C12N 1/00

(52) U.S. Cl. 536/23.1; 435/243; 435/320.1; 536/25.4

(57) ABSTRACT

The present invention generally relates to polypeptide tags and the purification of polypeptides, proteins, and protein fragments displaying such tags. More particularly, the invention is directed to novel polypeptides, nucleic acid sequences and vectors encoding novel polypeptides containing antigenic determinants. Also provided are methods and kits for using such polypeptides for the purification of target peptides, as well as methods of constructing vectors encoding the novel polypeptides and the novel polypeptides linked to a target peptide.

### RELATED APPLICATIONS

This application claims the benefit of U.S. Provisional Application Ser. No. 60/721,781, filed Sep. 29, 2005, and U.S. Provisional Application Ser. No. 60/722,786, filed Sep. 30, 2005, the entire content of each of which is hereby incorporated herein by reference.

### FIELD OF THE INVENTION

The present invention generally relates to polypeptide tags and the purification of polypeptides, proteins, and protein fragments displaying such tags. More particularly, the invention is directed to novel polypeptides and vectors encoding novel polypeptides containing antigenic determinants. Also provided are methods for using such polypeptides for the purification of target peptides, as well as methods of constructing vectors encoding the novel polypeptides and the novel polypeptides linked to a target peptide.

### BACKGROUND OF THE INVENTION

Various affinity purification protocols are currently employed to facilitate the isolation of fusion peptides and proteins. Affinity chromatography is based on the capacity of proteins to bind specifically and non-covalently with a ligand. Used alone, it can isolate proteins from very complex mixtures with not only a greater degree of purification than possible by sequential ion-exchange and gel column chromatography, but also without significant loss of activity. Typically, a ligand capable of binding with high specificity to an affinity matrix is chosen as the fusion partner. For example, p-aminophenyl-β-D-thiogalactosidyl-succinyldiaminohexyl-Sepharose selectively binds to β-galactosidase allowing the purification of β-gal fusion proteins. See Germino et al., Proc. Natl. Acad. Sci. USA 80:6848 (1983). Other expression systems which permit the affinity purification of fusion proteins include fusion proteins made with glutathione-S-transferase, which are selectively recovered on glutathione-agarose (Smith, D. B. and Johnson, K. S. Gene 67:31 (1988)); fusion proteins made with staphylococcal protein A, which are selectively recovered on IgG-Sepharose (Uhlen, M. et al. Gene 23:369 (1983)), fusion proteins made with calmodulin nickel binding proteins (CNBP), which are selectively recovered on calmodulin agarose (Stofko-Hahn, R. E. et al. FEBS Lett. 302(3):274-278); fusion proteins made with streptavidin, which are selectively recovered using biotin labeled beads or resins; fusion proteins made with biotinylated residues which are selectively recovered on avidin matrix ((Takashige, S. and Dale, G. L., Proc. Natl. Acad. Sci. USA. 85:1647-1651 (1988))); and fusion proteins made with the maltose-binding protein domain from the malE gene of E. coli, which are selectively recovered on amylose resins (Bach, H. et al., J. Mol. Biol. 312:79-93 (2001)).

Any number of these ligands may also be used in combination with one another. Examples include tandem affinity purification tags (sometimes referred to herein as TAPS or TAP tags) employing the calmodulin binding peptide (CBP) and the IgG binding domains of protein A (Rigaut, G. et al. Nat. Biotechnol., 17(10):1030-1032 (1999)) and calmodulin binding peptide, six histidine residues, and three copies of the hemagglutinin (HA) epitope (Honey, S. et al., Nuc. Acids Res. 29(4): e24 (2001)). These and other related TAPs, however, suffer from several limitations, including interference with complex assembly or protein function due to relatively large TAP tags, which are generally 20 KD or larger; a high rate of contamination with non-targeted endogenous proteins, thereby resulting in decreased purity of the isolated target protein; and the requirement of protease treatment for elution, which adds possible bacterial contaminants to the isolated target protein.

An alternative to the use of bulky ligands to detect and isolate proteins is the use of epitope tags. Epitope tagging utilizes antibodies against guest peptides to study protein localization at the cellular and subcellular levels. See, Kolodziej, P. A. and Young, R. A., Methods Enzymol. 194:508-519 (1991). Generally, these epitopes are fused to the amino or carboxy-terminus of the expressed protein making them more accessible to the antibody for detection and less likely to cause severe structural or functional perturbations. An epitope is exposed on the surface of the fusion protein, thereby making it available for recognition by the epitope-specific antibody and allowing for purification of proteins utilizing affinity purification techniques.

Like ligands, multiple copies of an epitope have been used to create fusion proteins with more than one copy of an epitope tag. U.S. Pat. No. 6,379,903 discloses the use of multiple copies of epitope tags, and in a particular embodiment, multiple copies of the FLAG® octapeptide, to detect and purify target proteins. Moreover, the multiple copies of the epitope tags may be combined with a metal affinity peptide to allow for co-purification using an antibody in combination with metal affinity chromatography. While these multiple epitope systems do not suffer from the same limitations of the multiple ligand systems discussed above, because multiple copies of the same epitope are typically used, co- or sequential purification using more than one antibody is typically not feasible.

### SUMMARY OF THE INVENTION

Thus, there exists a need for an epitope tag and expression system employing the use of such multiple and different epitope tags which would allow for co- or sequential purification, thereby increasing purity of a target protein, as well as increasing sensitivity and detection of recombinant peptides and proteins.

Briefly, therefore, among the aspects of the invention is a polypeptide, protein, or protein fragment comprising two or more antigenic domains. One aspect of the invention is a polypeptide, protein, or protein fragment represented by the formula R₁-Sp₁-Tag₁-Sp₂-R₂-Sp₃-Tag₂-Sp₄-R₃, wherein R₁ is hydrogen, a polypeptide, a protein, or a protein fragment; Sp₁ is a bond or a spacer comprising at least one amino acid residue; Tag₁ is an antigenic domain, the antigenic domain comprising at least one antigenic determinant selected from the group consisting of (i) a peptide sequence for which an antibody to the peptide sequence DYKDDDDK (SEQ ID NO: 1) has specificity, (ii) a peptide sequence for which an antibody to the peptide sequence DLYDDDDK (SEQ ID NO: 2) has specificity, (iii) the HA epitope, (iv) c-myc, (v) AcV5, and (vi) a peptide sequence having specificity for streptavidin or a streptavidin derivative; Sp₂ is a bond or a spacer comprising at least one amino acid residue; R₂ is a bond, a polypeptide, a protein, or a protein fragment; Sp₃ is a bond or a spacer comprising at least one amino acid; Tag₂ is an antigenic domain, said antigenic domain comprising at least one antigenic determinant selected from the group consisting of (i) a peptide sequence for which an antibody to the peptide sequence DYKDDDDK (SEQ ID NO: 1) has specificity, (ii) a peptide sequence for which an antibody to the peptide sequence DLYDDDDK (SEQ ID NO: 2) has specificity, (iii) the HA epitope, (iv) c-myc, (v) AcV5, and (vi) a peptide sequence having specificity for streptavidin or a streptavidin derivative; Sp₄ is a bond or a spacer comprising at least one amino acid; and R₃ is hydrogen, a polypeptide, a protein, or a protein fragment; wherein when Sp₂, R₂, and Sp₃ are each bonds Tag₁ and Tag₂ are not the same antigenic domain. Another aspect of the invention is a nucleic acid sequence encoding said polypeptide, protein, protein fragment.

Yet another aspect of the invention is a process for capturing a polypeptide, protein, or protein fragment of claim 1 from a sample, the process comprising: combining the sample with an antibody or receptor of Tag₁ to bind the polypeptide, protein, or protein fragment to the antibody or receptor; eluting the polypeptide, protein, protein fragment, or a portion thereof, from the antibody or receptor of Tag₁ to form a first eluant containing the polypeptide, protein, protein fragment, or a portion thereof; combining the first eluant with an antibody or receptor of Tag₂ to bind the polypeptide, protein, or protein fragment to the antibody or receptor; and eluting the polypeptide, protein, protein fragment, or a portion thereof, from the antibody or receptor of Tag₂ to form a second eluant containing the polypeptide, protein, or protein fragment, or a portion thereof.

Other objects and features will be in part apparent and in part pointed out hereinafter.

### BRIEF DESCRIPTION OF THE DRAWINGS

FIG. 1 is a schematic representation of the use of the FLAG® HA tandem affinity purification system, demonstrating the use of the tandem system to purify an endogenous “prey” protein from a proteinaceous sample using a “bait” protein tagged with both a FLAG® and HA epitope and co-purification as more fully described in Example 1. Tube 1 represents the starting material and contains the endogenous prey complex, contaminants, and bait protein. Tube 2 represents the of the EZView ANTI-FLAG® Resin. Tube 3 represents transfer of the wash resin. Tube 4 represents the first elution of the protein, complex. Tube 5 represents the binding of the ANTI-HA resin to the wash resin. Tube 6 represents the elution of the endogenous protein complex.

FIG. 2 is a schematic representation of the process for creating a fusion peptide comprising the FLAG® and HA epitope fused 5′ of a gene of interest as more fully described in Example 2. Referenced abbreviations are as follows: “GSP-F” is the gene specific primer-forward (not supplied); “R2” is the restriction enzyme overhang site 3′; “HA” is 20 of the HA epitope added to gene specific forward primer; “R1” is the restriction enzyme overhang 5′ (EcoRI, BamH I, or Xba I); “GSP-R” is the gene specific primer-reverse (not supplied); and “AP” is the anchor primer.

FIGS. 3A-3C demonstrate the ability of ANTI-FLAG® and anti-HA antibodies to bind to tandem-tagged proteins (tagged with both FLAG® and HA epitopes) as more fully described in Example 2. FIG. 3A is a gel demonstrating the incorporation of the FLAG®-HA tag into bacterial alkaline phosphotase (BAP). FIG. 3B discloses the nucleic acid and amino acid sequences of the FLAG®-HA tandem tag that was incorporated into BAP. FIG. 3C is a series of two gels demonstrating the ability of both ANTI-FLAG® and anti-HA antibodies (detected with anti-HA HRP) to bind to the tandem tagged FLAG®-HA BAP protein in different buffers ((buffers 1, 2, and 3).

FIGS. 4A-4C are a series of gels demonstrating the specificity of FLAG® and HA tags and the lack thereof of several other affinity resins as more fully described in Example 4. FIG. 4A is a series of two gels demonstrating affinity of the ANTI-FLAG® antibody and an anti-HA antibody for tandem tagged FLAG®-HA proteins in seven different plant species. FIG. 4B is a gel demonstrating the specificity of ANTI-FLAG® and anti-HA affinity resins in comparison to five other resins. FIG. 4C is a gel comparing the purity of a tandem tagged FLAG®-HA protein after single (either ANTI-FLAG® or anti-HA) immunoprecipitations to the purity of the same protein after consecutive immunoprecipitations (ANTI-FLAG® immunoprecipitation followed by anti-HA immunoprecipitation).

FIGS. 5A and 5B demonstrate the use of a tandem tagged FLAG®-HA p53 “bait” protein to isolate and purify an endogenous mammalian T antigen “prey” protein from a sample as more fully described in Example 5. FIG. 5A discloses a series of gels demonstrating the effect of eluting the bait and prey proteins using a series of elutions according to the experiments performed in Example 5. FIG. 5B discloses the peptide sequence (SEQ ID NO: 29) of the “prey” protein, confirmed by MS/MS to be the large tumor antigen T, isolated and purified according to the experiments performed in Example 5. Sequence coverage is 29%. Matched peptides are shown in bold.

FIG. 6 demonstrates the results of the use of a tandem tagged FLAG®-HA IAA1 “bait” protein to isolate and purify a c-myc labeled TIR1 “prey” protein expressed in Arabidopsis as more fully described in Example 6.

### DETAILED DESCRIPTION OF THE INVENTION

The present invention generally relates to processes for capturing recombinant polypeptides, proteins, or protein fragments containing multiple antigenic domains (sometimes referred to as a fusion peptide(s) or fusion protein(s)). Generally, this process comprises contacting a sample or liquid mixture comprising the recombinant polypeptide or protein, wherein the recombinant polypeptide or protein comprises one or more members of a binding pair, typically multiple antigenic domains, each domain comprising at least one antigenic determinant (sometimes referred to as an epitope or epitope tag), and has the sequence R₁-Sp₁-Tag₁-Sp₂-R₂-Sp₃-Tag₂-Sp₄-R₃ as defined below, with a complementary member of the binding pair, typically an antibody or receptor, to bind to one or more of the members of the binding pair contained in the recombinant polypeptide or protein. Generally, Tag₁ and Tag₂ may be a member of a binding pair, such as for example a ligand, a receptor, or an antigenic domain. Typically, however, Tag₁ and Tag₂ will be antigenic domains comprising at least one antigenic determinant that is non-cellularly derived. Moreover, such antigenic determinants will typically be short, comprising less than about 20 amino acids each. Typically, antibodies to the antigenic determinants may be immobilized, for example, on a solid support. The process may further comprise eluting the recombinant polypeptide or protein from the support. In certain embodiments, the process further comprises contacting the eluant with an antibody or receptor that binds one or more of the antigenic determinants contained in the recombinant polypeptide or protein. As mentioned above, the antibody or receptor may be immobilized. Moreover, the process may further comprise eluting the recombinant protein or polypeptide or a fragment thereof from the support. Often, the target polypeptide, protein, or protein fragment is a biologically active protein or protein fragment.

Alternatively, the recombinant polypeptide or protein may be isolated and purified by utilizing a solid support having immobilized metal ions to bind the recombinant protein or polypeptide. Specifically, the recombinant polypeptide or protein defined above may further comprise a metal ion-affinity peptide, sometimes referred to as a metal affinity tag (MAT or MAT tag), having the sequence His-Z₁-His-Arg-His-Z₂-His (SEQ ID NO: 35), wherein Z₁ is an amino acid residue selected from the group consisting of Ala, Arg, Asn, Asp, Gln, Glu, Ile, Lys, Phe, Pro, Ser, Thr, Trp, and Val; and Z₂ is an amino acid residue selected from the group consisting of Ala, Arg, Asn, Asp, Cys, Gln, Glu, Gly, Ile, Leu, Lys, Met, Pro, Ser, Thr, Tyr and Val. In addition to being identified, detected, isolated, captured, and/or purified by use of a solid support containing immobilized metal ions, a recombinant polypeptide or protein comprising the metal ion-affinity peptide sequence may also be identified, detected, isolated, captured, and/or purified using an antibody having specificity for the metal ion-affinity peptide as disclosed in U.S. application Ser. No. 11/128,486.

The Tandem Affinity Purification (TAP) Tags

Generally, Tag₁ and Tag₂ are members of a binding pair. For example, Tag₁ and Tag₂ may be a ligand, a receptor, an antigen, or an antibody. Examples of ligands and receptors are well known in the art, and include, for example, calmodulin binding peptide, streptavidin binding peptide, calmodulin and calmodulin derivatives, streptavidin and streptavidin derivatives, avidin and avidin derivatives, polyhistidine tag, polyarginine tag, antibodies and antigens, S-tag, cellulose binding domain, chitin-binding domain, glutathione S-transferase (GST), maltose-binding protein, TrxA, DsbA, hemagglutinin (HA) epitope, InaD, NorpA, GFP, strep I (AWRHPQFGG) (SEQ ID NO: 19), strep II (WSGPQFEK) (SEQ ID NO: 20), the SBP tag (Keefe, A. D. et al., Protein Expr. Purif. 23: 440-446 (2100)), S1 Aptamer (Li, Y. and Altman, S. Nuc. Acids Rec. 30(17): 3706-3711 (2002)), C-terminal streptavidin binding tag (Deka, R. et al., J. Biol. Chem. 277(44): 41857-41864 (2002)), and nano-tag (Lamla, T. and Erdmann, V A., Protein Expr. Purif. 33(1): 39-47 (2004)).

Regardless of whether Tag₁ or Tag₂ is a ligand, a receptor, an antigen, or an antibody, Tag₁ or Tag₂ may be a member of a binding pair that binds its complementary binding member (i.e., has a specificity for its complementary binding member) with at least a certain affinity. Generally, this affinity equates to a dissociation constant of at least about 1×10² M⁻¹, preferably at least about 1×10³ M⁻¹, more preferably at least about 1×10⁴ M⁻¹, still more preferably at least about 1×10⁵ M⁻¹, even more preferably at least about 1×10⁶ M⁻¹, still more preferably at least about 1×10⁹ M⁻¹, still more preferably at least about 1×10¹⁴ M⁻¹, even more preferably at least about 1×10¹⁵ M⁻¹, even more preferably at least about 1×10¹⁶ M⁻¹, still more preferably at least about 1×10²⁰ M⁻¹, or even greater.

Typically, Tag₁ and Tag₂ may comprise an antigenic domain comprising one or more antigenic determinants of the present invention. These antigenic determinants are typically non-cellularly derived. Generally, such antigenic determinants are synthetic nucleic acid or polypeptide sequences or fragments thereof or are nucleic acids or polypeptide sequences present in only the genomes of viruses or other non-cellular organisms. These antigenic domains should not have a high affinity to plant and mammalian proteins, thereby allowing for the use of such polypeptides, proteins, or protein fragments for the isolation of plant and mammalian proteins with minimal non-specific or undesired binding of the polypeptides, proteins, or protein fragments of the invention by antibodies endogenous to plant and mammalian cells.

Such antigenic determinants are generally also of minimal sequence length, generally being about 15 amino acids or less, preferably about 10 amino acids or less, and more preferably about 8 amino acids or less. Accordingly, for example, the antigenic domains will typically be about 45 amino acids or less if the antigenic domain comprises about three antigenic determinants and about 30 amino acids or less if the antigenic domain comprises about two antigenic determinants. Moreover, hydrophilic amino acids are preferred as they are more likely to be exposed on the protein surface thus resulting in increased accessibility to the antibody. See Hopp T. P. and Woods K. R., Proc. Natl. Acad. Sci. 78: 3824-3828 (1981). Accordingly, the antigenic determinants are typically hydrophilic, as generally about 50%, more preferably about 60%, even more preferably about 75%, and still more preferably about 80% of the residues of the antigenic determinants are hydrophilic residues. Optionally, however, the antigenic determinants may include one or more non-aromatic, hydrophobic residues.

Examples of such antigenic determinants include, for example, peptide sequences having affinity for the M1 (ATCC HB 9259), M2 and/or M5 antibodies, including for example, the FLAG® octapeptide (DYKDDDDK) (SEQ ID NO: 1), multiple copies of the FLAG® octapeptide, such as, for example, DYKDDDDKDYKDDDDK (SEQ ID NO: 25), DYKDDDDKDYKDDDDKDYKDDDDK (SEQ ID NO: 26), DYKDHDGDYKDDDDK (SEQ ID NO: 27), DYDDHDIDYKDDDDK (SEQ ID NO: 28), DYKDHDGDYKDHDIDYKDDDDK (SEQ ID NO: 6), and MDYKDHDGDYKDHDIDYKDDDDK (SEQ ID NO: 13), and multi-copy FLAG® peptides represented by the formulae disclosed below; peptide sequences having affinity for antibodies to the XPRESS® octapeptide (DLYDDDDK) (SEQ ID NO: 2), multiple copies of the XPRESS® octapeptide, such as for example, DLYDDDDKDLYDDDDK (SEQ ID NO: 30), DLYDDDDKDLYDDDDKDLYDDDDK (SEQ ID NO: 31), DLYDHDGDLYDDDDK (SEQ ID NO: 32), DLYDHDIDLYDDDDK (SEQ ID NO: 33), DLYDHDGDLYDHDIDLYDDDDK (SEQ ID NO: 7), and MDLYDHDGDLYDHDIDLYDDDDK (SEQ ID NO: 14), and multi-copy XPRESS™ peptides represented by the formulae disclosed below; the hemagglutinin (HA) epitope (YPYDVPDYA) (SEQ ID NO: 3); AcV5 (SWKDASGWS) (SEQ ID NO: 4); c-myc (EQKLISEEDL) (SEQ ID NO: 5); and peptide sequences having affinity for streptavidin or streptavidin derivatives, such as, for example, strep I (AWRHPQFGG) (SEQ ID NO: 19), and strep II (WSGPQFEK) (SEQ ID NO: 20), the SBP tag (Keefe, A. D. et al., Protein Expr. Purif. 23: 440-446 (2100)), S1 Aptamer (Li, Y. and Altman, S., Nuc. Acids Rec. 30(17): 3706-3711 (2002)), C-terminal streptavidin binding tag (Deka, R. et al., J. Biol. Chem. 277(44): 41857-41864 (2002)), and nano-tag (Lamla, T. and Erdmann, V A., Protein Expr. Purif. 33(1): 39-47 (2004)). Particularly preferred antigenic determinants include the FLAG® octapeptide, the XPRESS™ octapeptide, the HA epitope, c-myc, and AcV5, and multiple copies thereof.

One aspect of the invention, therefore, is a recombinant polypeptide, protein, or protein fragment comprising 5′ to 3′ two antigenic domains and a polypeptide, a protein, or a protein fragment. In one embodiment, the recombinant polypeptide, protein, or protein fragment has the sequence R₁-Sp₁-Tag₁-Sp₂-R₂-Sp₃-Tag₂-Sp₄-R₃, wherein R₁ is hydrogen; Sp₁ is a bond or a spacer comprising at least one amino acid residue; Tag₁ is a first antigenic domain, said antigenic domain comprising at least one antigenic determinant selected from the group consisting of a peptide sequence having affinity for the M1 antibody, a peptide sequence having affinity for the M2 antibody, a peptide sequence having affinity for the M5 antibody, a peptide sequence having affinity for the peptide sequence DYKDDDDK (SEQ ID NO: 1), a peptide sequence having affinity for the peptide sequence DLYDDDDK (SEQ ID NO: 2), the HA epitope, c-myc, AcV5, and a peptide sequence having affinity for streptavidin or streptavidin derivatives; Sp₂ is a bond or a spacer comprising at least one amino acid residue; R₂ is a bond; Sp₃ is a bond or a spacer comprising at least one amino acid; Tag₂ is a second antigenic domain, said antigenic domain comprising at least one antigenic determinant selected from the group consisting of a peptide sequence having affinity for the M1 antibody; a peptide sequence having affinity for the M2 antibody, a peptide sequence having affinity for the M5 antibody, a peptide sequence having affinity for the peptide sequence DYKDDDDK (SEQ ID NO: 1), a peptide sequence having affinity for the peptide sequence DLYDDDDK (SEQ ID NO: 2), the HA epitope, c-myc, AcV5, and a peptide sequence having affinity for streptavidin or streptavidin derivatives; Sp₄ is a bond or a spacer comprising at least one amino acid; and R₃ is a polypeptide, a protein, or a protein fragment.

Another aspect of the invention is a recombinant polypeptide, protein, or protein fragment comprising 5′ to 3′ a polypeptide, a protein, or a protein fragment and two antigenic domains. In one embodiment, the recombinant polypeptide, protein or protein fragment has the sequence R₁-Sp₁-Tag₁-Sp₂-R₂-Sp₃-Tag₂-Sp₄-R₃, wherein R₁ is a polypeptide, a protein, or a protein fragment; Sp₁ is a bond or a spacer comprising at least one amino acid residue; Tag₁ is a first antigenic domain, said antigenic domain comprising at least one antigenic determinant selected from the group consisting of a peptide sequence having affinity for the M1 antibody; a peptide sequence having affinity for the M2 antibody, a peptide sequence having affinity for the M5 antibody, a peptide sequence having affinity for the peptide sequence DYKDDDDK (SEQ ID NO: 1), a peptide sequence having affinity for the peptide sequence DLYDDDDK (SEQ ID NO: 2), the HA epitope, c-myc, AcV5, and a peptide sequence having affinity for streptavidin or streptavidin derivatives; Sp₂ is a bond or a spacer comprising at least one amino acid residue; R₂ is a bond; Sp₃ is a bond or a spacer comprising at least one amino acid; Tag₂ is a second antigenic domain, said antigenic domain comprising at least one antigenic determinant selected from the group consisting of a peptide sequence having affinity for the M1 antibody; a peptide sequence having affinity for the M2 antibody, a peptide sequence having affinity for the M5 antibody, a peptide sequence having affinity for the peptide sequence DYKDDDDK (SEQ ID NO: 1), a peptide sequence having affinity for the peptide sequence DLYDDDDK (SEQ ID NO: 2), the HA epitope, c-myc, AcV5, and a peptide sequence having affinity for streptavidin or streptavidin derivatives; Sp₄ is a bond or a spacer comprising at least one amino acid; and R₃ is hydrogen.

Another aspect of the present invention is a recombinant polypeptide, protein, or protein fragment comprising 5′ to 3′ a first antigenic domain, a polypeptide, protein, or protein fragment, and a second antigenic domain. In one embodiment, the recombinant polypeptide, protein, or protein fragment has the sequence R₁-Sp₁-Tag₁-Sp₂-R₂-Sp₃-Tag₂-Sp₄-R₃, wherein R₁ is hydrogen; Sp₁ is a bond or a spacer comprising at least one amino acid residue; Tag₁ is a first antigenic domain, said antigenic domain comprising at least one antigenic determinant selected from the group consisting of a peptide sequence having affinity for the M1 antibody; a peptide sequence having affinity for the M2 antibody, a peptide sequence having affinity for the M5 antibody, a peptide sequence having affinity for the peptide sequence DYKDDDDK (SEQ ID NO: 1), a peptide sequence having affinity for the peptide sequence DLYDDDDK (SEQ ID NO: 2), the HA epitope, c-myc, AcV5, and a peptide sequence having affinity for streptavidin or streptavidin derivatives; Sp₂ is a bond or a spacer comprising at least one amino acid residue; R₂ is a polypeptide, a protein, or a protein fragment; Sp₃ is a bond or a spacer comprising at least one amino acid; Tag₂ is a second antigenic domain, said antigenic domain comprising at least one antigenic determinant selected from the group consisting of a peptide sequence having affinity for the M1 antibody, a peptide sequence having affinity for the M2 antibody, a peptide sequence having affinity for the M5 antibody, a peptide sequence having affinity for the peptide sequence DYKDDDDK (SEQ ID NO: 1), a peptide sequence having affinity for the peptide sequence DLYDDDDK (SEQ ID NO: 2), the HA epitope, c-myc, AcV5, and a peptide sequence having affinity for streptavidin or streptavidin derivatives; Sp₄ is a bond or a spacer comprising at least one amino acid; and R₃ is hydrogen.

With respect to these embodiments of the invention, preferably Tag₁ is a first antigenic domain, said first antigenic domain comprising at least one antigenic determinant independently selected from the group consisting of the FLAG® octapeptide, the XPRESS™ octapeptide, the HA epitope, AcV5, c-myc, strep 1, and strep II and Tag₂ is a second antigenic domain, said second antigenic domain comprising at least one antigenic determinant independently selected from the group consisting of the FLAG® octapeptide, the XPRESS™ octapeptide, the HA epitope, AcV5, c-myc, strep 1, and strep II. In one particular embodiment, Tag₁ is the FLAG® octapeptide and Tag₂ is the XPRESS™ octapeptide. In another particular embodiment, Tag₁ is the FLAG® octapeptide and Tag₂ is the HA epitope. In yet another embodiment, Tag₁ is the FLAG® octapeptide and Tag₂ is AcV5. In still another particular embodiment, Tag₁ is the XPRESS™ octapeptide and Tag₂ is the FLAG® octapeptide. In yet another particular embodiment, Tag₁ is the HA epitope and Tag₂ is the FLAG® octapeptide. In still another particular embodiment, Tag₁ is AcV5 and Tag₂ is the FLAG® octapeptide. Generally, when Sp₂, R₂, and Sp₃ are each bonds Tag₁ and Tag₂ are not the same antigenic domain.

The antigenic domains may also comprise more than a single antigenic determinant. In such embodiments, each of the antigenic domains may comprise two, three, four, five, or more copies of an antigenic determinant. Each of the antigenic domains may independently comprise, for example, two, three, four, five, or more copies of the FLAG® octapeptide, the XPRESS™ octapeptide, the hemagglutinin (HA) epitope, AcV5, c-myc, strep 1, and strep II; more preferably, two, three, four, five, or more copies of the FLAG® octapeptide, the XPRESS™ octapeptide, the HA epitope, c-myc, and AcV5; still more preferably, two, three, four, five, or more copies of the FLAG® octapeptide, the HA epitope, and AcV5; and even more preferably, two, three, four, five, or more copies of the FLAG® octapeptide and the HA epitope. In a particularly preferred embodiment, each of the antigenic domains may comprise the 3×FLAG® peptide sequence DYKDHDGDYKDHDIDYKDDDDK (SEQ ID NO: 6), the peptide sequence DLYDHDGDLYDHDIDLYDDDDK (SEQ ID NO: 7), the peptide sequence YPYDVPDYAYPYDVPDYA (SEQ ID NO. 8), the peptide sequence YPYDVPDYAYPYDVPDYAYPYDVPDYA (SEQ ID NO: 9), the peptide sequence SWKDASGWSSWKDASGWS (SEQ ID NO. 10), or the peptide sequence SWKDASGWSSWKDASGWSSWKDASGWS (SEQ ID NO: 11).

One aspect of the invention, therefore, is a recombinant polypeptide, protein, or protein fragment comprising 5′ to 3′ two antigenic domains and a polypeptide, a protein, or a protein fragment. In one embodiment, the recombinant polypeptide, protein, or protein fragment has the sequence R₁-Sp₁-Tag₁-Sp₂-R₂-Sp₃-Tag₂-Sp₄-R₃, wherein R₁ is hydrogen; Sp₁ is a bond or a spacer comprising at least one amino acid residue; Tag₁ is a first antigenic domain independently selected from the group consisting of the peptide sequence DYKDHDGDYKDHDIDYKDDDDK (SEQ ID NO: 6), the peptide sequence DLYDHDGDLYDHDIDLYDDDDK (SEQ ID NO: 7), the peptide sequence YPYDVPDYAYPYDVPDYA (SEQ ID NO. 8), the peptide sequence YPYDVPDYAYPYDVPDYAYPYDVPDYA (SEQ ID NO: 9), the peptide sequence SWKDASGWSSWKDASGWS (SEQ ID NO. 10), the peptide sequence SWKDASGWSSWKDASGWSSWKDASGWS (SEQ ID NO: 11), the peptide sequence AWRHPQFGGAWRHPQFGG (SEQ ID NO: 21), the peptide sequence AWRHPQFGGAWRHPQFGGAWRHPQFGG (SEQ ID NO: 22) WSGPQFEKWSGPQFEK (SEQ ID NO: 23), and WSGPQFEKWSGPQFEKWSGPQFEK (SEQ ID NO: 24); Sp₂ is a bond or a spacer comprising at least one amino acid residue; R₂ is a bond; Sp₃ is a bond or a spacer comprising at least one amino acid; Tag₂ is a second antigenic domain independently selected from the group consisting of the peptide sequence DLYDHDGDLYDHDIDLYDDDDK (SEQ ID NO: 7), the peptide sequence YPYDVPDYAYPYDVPDYA (SEQ ID NO. 8), the peptide sequence YPYDVPDYAYPYDVPDYAYPYDVPDYA (SEQ ID NO: 9), the peptide sequence SWKDASGWSSWKDASGWS (SEQ ID NO. 10), the peptide sequence SWKDASGWSSWKDASGWSSWKDASGWS (SEQ ID NO: 11), the peptide sequence AWRHPQFGGAWRHPQFGG (SEQ ID NO: 21), the peptide sequence AWRHPQFGGAWRHPQFGGAWRHPQFGG (SEQ ID NO: 22) WSGPQFEKWSGPQFEK (SEQ ID NO: 23), and WSGPQFEKWSGPQFEKWSGPQFEK (SEQ ID NO: 24); Sp₄ is a bond or a spacer comprising at least one amino acid; and R₃ is a polypeptide, a protein, or a protein fragment.

Another aspect of the invention is a recombinant polypeptide, protein, or protein fragment comprising 5′ to 3′ a polypeptide, a protein, or a protein fragment and two antigenic domains. In one embodiment, the recombinant polypeptide, protein or protein fragment has the sequence R₁-Sp₁-Tag₁-Sp₂-R₂-Sp₃-Tag₂-Sp₄-R₃, wherein R₁ is a polypeptide, a protein, or a protein fragment; Sp₁ is a bond or a spacer comprising at least one amino acid residue Tag₂ is a second antigenic domain independently selected from the group consisting of the peptide sequence DLYDHDGDLYDHDIDLYDDDDK (SEQ ID NO: 7), the peptide sequence YPYDVPDYAYPYDVPDYA (SEQ ID NO. 8), the peptide sequence YPYDVPDYAYPYDVPDYAYPYDVPDYA (SEQ ID NO: 9), the peptide sequence SWKDASGWSSWKDASGWS (SEQ ID NO. 10), the peptide sequence SWKDASGWSSWKDASGWSSWKDASGWS (SEQ ID NO: 11), the peptide sequence AWRHPQFGGAWRHPQFGG (SEQ ID NO: 21), the peptide sequence AWRHPQFGGAWRHPQFGGAWRHPQFGG (SEQ ID NO: 22) WSGPQFEKWSGPQFEK (SEQ ID NO: 23), and WSGPQFEKWSGPQFEKWSGPQFEK (SEQ ID NO: 24); Sp₂ is a bond or a spacer comprising at least one amino acid residue; R₂ is a bond; Sp₃ is a bond or a spacer comprising at least one amino acid; Tag₂ is a second antigenic domain independently selected from the group consisting of the peptide sequence DLYDHDGDLYDHDIDLYDDDDK (SEQ ID NO: 7), the peptide sequence YPYDVPDYAYPYDVPDYA (SEQ ID NO. 8), the peptide sequence YPYDVPDYAYPYDVPDYAYPYDVPDYA (SEQ ID NO: 9), the peptide sequence SWKDASGWSSWKDASGWS (SEQ ID NO: 10), the peptide sequence SWKDASGWSSWKDASGWSSWKDASGWS (SEQ ID NO: 11), the peptide sequence AWRHPQFGGAWRHPQFGG (SEQ ID NO: 21), the peptide sequence AWRHPQFGGAWRHPQFGGAWRHPQFGG (SEQ ID NO: 22) WSGPQFEKWSGPQFEK (SEQ ID NO: 23), and WSGPQFEKWSGPQFEKWSGPQFEK (SEQ ID NO: 24); Sp₄ is a bond or a spacer comprising at least one amino acid; and R₃ is hydrogen.

Another aspect of the present invention is a recombinant polypeptide, protein, or protein fragment comprising 5′ to 3′ a first antigenic domain, a polypeptide, protein, or protein fragment, and a second antigenic domain. In one embodiment, the recombinant polypeptide, protein, or protein fragment has the sequence R₁-Sp₁-Tag₁-Sp₂-R₂-Sp₃-Tag₂-Sp₄-R₃, wherein R₁ is hydrogen; Sp₁ is a bond or a spacer comprising at least one amino acid residue; Tag₁ is a first antigenic domain independently selected from the group consisting of the peptide sequence DLYDHDGDLYDHDIDLYDDDDK (SEQ ID NO: 7), the peptide sequence YPYDVPDYAYPYDVPDYA (SEQ ID NO. 8), the peptide sequence YPYDVPDYAYPYDVPDYAYPYDVPDYA (SEQ ID NO: 9), the peptide sequence SWKDASGWSSWKDASGWS (SEQ ID NO. 10), the peptide sequence SWKDASGWSSWKDASGWSSWKDASGWS (SEQ ID NO: 11), the peptide sequence AWRHPQFGGAWRHPQFGG (SEQ ID NO: 21), the peptide sequence AWRHPQFGGAWRHPQFGGAWRHPQFGG (SEQ ID NO: 22) WSGPQFEKWSGPQFEK (SEQ ID NO: 23), and WSGPQFEKWSGPQFEKWSGPQFEK (SEQ ID NO: 24); Sp₂ is a bond or a spacer comprising at least one amino acid residue; R₂ is a polypeptide, a protein, or a protein fragment; Sp₃ is a bond or a spacer comprising at least one amino acid; Tag₂ is a second antigenic domain selected from the group consisting of the peptide sequence DLYDHDGDLYDHDIDLYDDDDK (SEQ ID NO: 7), the peptide sequence YPYDVPDYAYPYDVPDYA (SEQ ID NO. 8), the peptide sequence YPYDVPDYAYPYDVPDYAYPYDVPDYA (SEQ ID NO: 9), the peptide sequence SWKDASGWSSWKDASGWS (SEQ ID NO. 10), the peptide sequence SWKDASGWSSWKDASGWSSWKDASGWS (SEQ ID NO: 11), the peptide sequence AWRHPQFGGAWRHPQFGG (SEQ ID NO: 21), the peptide sequence AWRHPQFGGAWRHPQFGGAWRHPQFGG (SEQ ID NO: 22) WSGPQFEKWSGPQFEK (SEQ ID NO: 23), and WSGPQFEKWSGPQFEKWSGPQFEK (SEQ ID NO: 24); Sp₄ is a bond or a spacer comprising at least one amino acid; and R₃ is hydrogen.

With respect to these embodiments of the invention, preferably Tag₁ and Tag₂ are selected from the group consisting of DYKDHDGDYKDHDIDYKDDDDK (SEQ ID NO. 6), DLYDHDGDLYDHDIDLYDDDDK (SEQ ID NO: 7), YPYDVPDYAYPYDVPDYAYPYDVPDYA (SEQ ID NO: 9), is SWKDASGWSSWKDASGWSSWKDASGWS (SEQ ID NO: 11), In one particular embodiment, Tag₁ is DYKDHDGDYKDHDIDYKDDDDK (SEQ ID NO. 6) and Tag₂ is DLYDHDGDLYDHDIDLYDDDDK (SEQ ID NO: 7). In another particular embodiment, Tag₁ is DYKDHDGDYKDHDIDYKDDDDK (SEQ ID NO. 6) and Tag₂ is YPYDVPDYAYPYDVPDYAYPYDVPDYA (SEQ ID NO: 9). In yet another embodiment, Tag₁ is DYKDHDGDYKDHDIDYKDDDDK (SEQ ID NO. 6) and Tag₂ is SWKDASGWSSWKDASGWSSWKDASGWS (SEQ ID NO: 11). In still another embodiment, Tag₁ is DLYDHDGDLYDHDIDLYDDDDK (SEQ ID NO: 7) and Tag₂ is DYKDHDGDYKDHDIDYKDDDDK (SEQ ID NO. 6). In yet another embodiment, Tag₁ is YPYDVPDYAYPYDVPDYAYPYDVPDYA (SEQ ID NO:9) and Tag₂ is DYKDHDGDYKDHDIDYKDDDDK (SEQ ID NO. 6). In another embodiment, Tag₁ is SWKDASGWSSWKDASGWSSWKDASGWS (SEQ ID NO: 11) and Tag₂ is DYKDHDGDYKDHDIDYKDDDDK (SEQ ID NO. 6).

Further examples of combinations of Tag₁ and Tag₂ that may be used in the polypeptides, proteins, or protein fragments (or nucleic acids encoding the same) of the present invention are list in Table I.

TABLE I
Tag₁/Tag₂ Combinations

| | | |
|---|---|---|
| Combo # | Tag₁ | Tag₂ |

| | | |
|---|---|---|
| 1 | DYKDDDDK | DLYDDDDK |
| | (SEQ ID NO: 1) | (SEQ ID NO: 2) |
| 2 | DYKDDDDK | YPYDVPDYA |
| | (SEQ ID NO: 1) | (SEQ ID NO: 3) |
| 3 | DYKDDDDK | SWKDASGWS |
| | (SEQ ID NO: 1) | (SEQ ID NO: 4) |
| 4 | DYKDDDDK | EQKLISEEDL |
| | (SEQ ID NO: 1) | (SEQ ID NO: 5) |
| 5 | DYKDDDDK | AWRHPQFGG |
| | (SEQ ID NO: 1) | (SEQ ID NO: 19) |
| 6 | DYKDDDDK | WSGPQFEK |
| | (SEQ ID NO: 1) | (SEQ ID NO: 20) |
| 7 | DYKDDDDK | DLYDDDDKDLYDDDDK |
| | (SEQ ID NO: 1) | (SEQ ID NO: 30) |
| 8 | DYKDDDDK | YPYDVPDYAYPYDVPDYA |
| | (SEQ ID NO: 1) | (SEQ ID NO: 8) |
| 9 | DYKDDDDK | SWKDASGWSSWKDASGWS |
| | (SEQ ID NO: 1) | SEQ ID NO: 10) |
| 10 | DYKDDDDK | EQKLISEEDLEQKLISEEDL |
| | (SEQ ID NO: 1) | (SEQ ID NO: 36) |
| 11 | DYKDDDDK | AWRHPQFGGAWRHPQFGG |
| | (SEQ ID NO: 1) | (SEQ ID NO: 21) |
| 12 | DYKDDDDK | WSGPQFEKWSGPQFEK |
| | (SEQ ID NO: 1) | (SEQ ID NO: 23) |
| 13 | DYKDDDDK | DLYDDDDKDLYDDDDKDLYDDDDK |
| | (SEQ ID NO: 1) | (SEQ ID NO: 31) |
| 14 | DYKDDDDK | YPYDVPDYAYPYDVPDYAYPYDVPDYA |
| | (SEQ ID NO: 1) | 9SEQ ID NO: 9) |
| 15 | DYKDDDDK | SWKDASGWSSWKDASGWSSWKDASGWS |
| | (SEQ ID NO: 1) | (SEQ ID NO: 11) |
| 16 | DYKDDDDK | EQKLISEEDLEQKLISEEDLEQKLISEEDL |
| | (SEQ ID NO: 1) | (SEQ ID NO: 37) |
| 17 | DYKDDDDK | AWRHPQFGGAWRHPQFGGAWRHPQFGG |
| | (SEQ ID NO: 1) | (SEQ ID NO: 22) |
| 18 | DYKDDDDK | WSGPQFEKWSGPQFEKWSGPQFEK |
| | (SEQ ID NO: 1) | (SEQ ID NO: 24) |
| 19 | DYKDDDDK | DLYDHDGDLYDHDIDLYDDDDK |
| | (SEQ ID NO: 1) | (SEQ ID NO: 7) |
| 20 | DYKDDDDKDYKDDDDK | DLYDDDDK |
| | (SEQ ID NO: 25) | (SEQ ID NO: 2) |
| 21 | DYKDDDDKDYKDDDDK | YPYDVPDYA |
| | (SEQ ID NO: 25) | (SEQ ID NO: 3) |
| 22 | DYKDDDDKDYKDDDDK | SWKDASGWS |
| | (SEQ ID NO: 25) | (SEQ ID NO: 4) |
| 23 | DYKDDDDKDYKDDDDK | EQKLISEEDL |
| | (SEQ ID NO: 25) | (SEQ ID NO: 5) |
| 24 | DYKDDDDKDYKDDDDK | AWRHPQFGG |
| | (SEQ ID NO: 25) | (SEQ ID NO: 19) |
| 25 | DYKDDDDKDYKDDDDK | WSGPQFEK |
| | (SEQ ID NO: 25) | (SEQ ID NO: 20) |
| 26 | DYKDDDDKDYKDDDDK | DLYDDDDKDLYDDDDK |
| | (SEQ ID NO: 25) | (SEQ ID NO: 30) |
| 27 | DYKDDDDKDYKDDDDK | YPYDVPDYAYPYDVPDYA |
| | (SEQ ID NO: 25) | (SEQ ID NO: 8) |
| 28 | DYKDDDDKDYKDDDDK | SWKDASGWSSWKDASGWS |
| | (SEQ ID NO: 25) | (SEQ ID NO: 10) |
| 29 | DYKDDDDKDYKDDDDK | EQKLISEEDLEQKLISEEDL |
| | (SEQ ID NO: 25) | (SEQ ID NO: 36) |
| 30 | DYKDDDDKDYKDDDDK | AWRHPQFGGAWRHPQFGG |
| | (SEQ ID NO: 25) | (SEQ ID NO: 21) |
| 31 | DYKDDDDKDYKDDDDK | WSGPQFEKWSGPQFEK |
| | (SEQ ID NO: 25) | (SEQ ID NO: 23) |
| 32 | DYKDDDDKDYKDDDDK | DLYDDDDKDLYDDDDKDLYDDDDK |
| | (SEQ ID NO: 25) | (SEQ ID NO: 31) |
| 33 | DYKDDDDKDYKDDDDK | YPYDVPDYAYPYDVPDYAYPYDVPDYA |
| | (SEQ ID NO: 25) | (SEQ ID NO: 9) |
| 34 | DYKDDDDKDYKDDDDK | SWKDASGWSSWKDASGWSSWKDASGWS |
| | (SEQ ID NO: 25) | (SEQ ID NO: 11) |
| 35 | DYKDDDDKDYKDDDDK | EQKLISEEDLEQKLISEEDLEQKLISEEDL |
| | (SEQ ID NO: 25) | (SEQ ID NO: 37) |
| 36 | DYKDDDDKDYKDDDDK | AWRHPQFGGAWRHPQFGGAWRHPQFGG |
| | (SEQ ID NO: 25) | (SEQ ID NO: 22) |
| 37 | DYKDDDDKDYKDDDDK | WSGPQFEKWSGPQFEKWSGPQFEK |
| | (SEQ ID NO: 25) | (SEQ ID NO: 24) |
| 38 | DYKDDDDKDYKDDDDK | DLYDHDGDLYDHDIDLYDDDDK |
| | (SEQ ID NO: 25) | (SEQ ID NO: 7) |
| 39 | DYKDDDDKDYKDDDDKDYKDDDDK | DLYDDDDK |
| | (SEQ ID NO: 26) | (SEQ ID NO: 2) |
| 40 | DYKDDDDKDYKDDDDKDYKDDDDK | YPYDVPDYA |
| | (SEQ ID NO: 26) | (SEQ ID NO: 3) |
| 41 | DYKDDDDKDYKDDDDKDYKDDDDK | SWKDASGWS |
| | (SEQ ID NO: 26) | (SEQ ID NO: 4) |
| 42 | DYKDDDDKDYKDDDDKDYKDDDDK | EQKLISEEDL |
| | (SEQ ID NO: 26) | )SEQ ID NO: 5) |
| 43 | DYKDDDDKDYKDDDDKDYKDDDDK | AWRHPQFGG |
| | (SEQ ID NO: 26) | (SEQ ID NO: 19) |
| 44 | DYKDDDDKDYKDDDDKDYKDDDDK | WSGPQFEK |
| | (SEQ ID NO: 26) | (SEQ ID NO: 20) |
| 45 | DYKDDDDKDYKDDDDKDYKDDDDK | DLYDDDDKDLYDDDDK |
| | (SEQ ID NO: 26) | (SEQ ID NO: 30) |
| 46 | DYKDDDDKDYKDDDDKDYKDDDDK | YPYDVPDYAYPYDVPDYA |
| | (SEQ ID NO: 26) | (SEQ ID NO: 8) |
| 47 | DYKDDDDKDYKDDDDKDYKDDDDK | SWKDASGWSSWKDASGWS |
| | (SEQ ID NO: 26) | (SEQ ID NO: 10) |
| 48 | DYKDDDDKDYKDDDDKDYKDDDDK | EQKLISEEDLEQKLISEEDL |
| | (SEQ ID NO: 26) | (SEQ ID NO: 36) |
| 49 | DYKDDDDKDYKDDDDKDYKDDDDK | AWRHPQFGGAWRHPQFGG |
| | (SEQ ID NO: 26) | (SEQ ID NO: 21) |
| 50 | DYKDDDDKDYKDDDDKDYKDDDDK | WSGPQFEKWSGPQFEK |
| | (SEQ ID NO: 26) | (SEQ ID NO: 23) |
| 51 | DYKDDDDKDYKDDDDKDYKDDDDK | DLYDDDDKDLYDDDDKDLYDDDDK |
| | (SEQ ID NO: 26) | (SEQ ID NO: 31) |
| 52 | DYKDDDDKDYKDDDDKDYKDDDDK | YPYDVPDYAYPYDVPDYAYPYDVPDYA |
| | (SEQ ID NO: 26) | (SEQ ID NO: 9) |
| 53 | DYKDDDDKDYKDDDDKDYKDDDDK | SWKDASGWSSWKDASGWSSWKDASGWS |
| | (SEQ ID NO: 26) | (SEQ ID NO: 11) |
| 54 | DYKDDDDKDYKDDDDKDYKDDDDK | EQKLISEEDLEQKLISEEDLEQKLISEEDL |
| | (SEQ ID NO: 26) | (SEQ ID NO: 37) |
| 55 | DYKDDDDKDYKDDDDKDYKDDDDK | AWRHPQFGGAWRHPQFGGAWRHPQFGG |
| | (SEQ ID NO: 26) | (SEQ ID NO: 22) |
| 56 | DYKDDDDKDYKDDDDKDYKDDDDK | WSGPQFEKWSGPQFEKWSGPQFEK |
| | (SEQ ID NO: 26) | (SEQ ID NO: 24) |
| 57 | DYKDDDDKDYKDDDDKDYKDDDDK | DLYDHDGDLYDHDIDLYDDDDK |
| | (SEQ ID NO: 26) | (SEQ ID NO: 7) |
| 58 | DYKDHDGDYKDDDDK | DLYDDDDK |
| | (SEQ ID NO: 27) | (SEQ ID NO: 2) |
| 59 | DYKDHDGDYKDDDDK | YPYDVPDYA |
| | (SEQ ID NO: 27) | (SEQ ID NO: 3) |
| 60 | DYKDHDGDYKDDDDK | SWKDASGWS |
| | (SEQ ID NO: 27) | (SEQ ID NO: 4) |
| 61 | DYKDHDGDYKDDDDK | EQKLISEEDL |
| | (SEQ ID NO: 27) | (SEQ ID NO: 5) |
| 62 | DYKDHDGDYKDDDDK | AWRHPQFGG |
| | (SEQ ID NO: 27) | (SEQ ID NO: 19) |
| 63 | DYKDHDGDYKDDDDK | WSGPQFEK |
| | (SEQ ID NO: 27) | (SEQ ID NO: 20) |
| 64 | DYKDHDGDYKDDDDK | DLYDDDDKDLYDDDDK |
| | (SEQ ID NO: 27) | (SEQ ID NO: 30) |
| 65 | DYKDHDGDYKDDDDK | YPYDVPDYAYPYDVPDYA |
| | (SEQ ID NO: 27) | 9SEQ ID NO: 8) |
| 66 | DYKDHDGDYKDDDDK | SWKDASGWSSWKDASGWS |
| | (SEQ ID NO: 27) | (SEQ ID NO: 10) |
| 67 | DYKDHDGDYKDDDDK | EQKLISEEDLEQKLISEEDL |
| | (SEQ ID NO: 27) | (SEQ ID NO: 36) |
| 68 | DYKDHDGDYKDDDDK | AWRHPQFGGAWRHPQFGG |
| | (SEQ ID NO: 27) | (SEQ ID NO: 21) |
| 69 | DYKDHDGDYKDDDDK | WSGPQFEKWSGPQFEK |
| | (SEQ ID NO: 27) | (SEQ ID NO: 23) |
| 70 | DYKDHDGDYKDDDDK | DLYDDDDKDLYDDDDKDLYDDDDK |
| | (SEQ ID NO: 27) | (SEQ ID NO: 31) |
| 71 | DYKDHDGDYKDDDDK | YPYDVPDYAYPYDVPDYAYPYDVPDYA |
| | (SEQ ID NO: 27) | (SEQ ID NO: 9) |
| 72 | DYKDHDGDYKDDDDK | SWKDASGWSSWKDASGWSSWKDASGWS |
| | (SEQ ID NO: 27) | (SEQ ID NO: 11) |
| 73 | DYKDHDGDYKDDDDK | EQKLISEEDLEQKLISEEDLEQKLISEEDL |
| | (SEQ ID NO: 27) | (SEQ ID NO: 37) |
| 74 | DYKDHDGDYKDDDDK | AWRHPQFGGAWRHPQFGGAWRHPQFGG |
| | (SEQ ID NO: 27) | (SEQ ID NO: 22) |
| 75 | DYKDHDGDYKDDDDK | WSGPQFEKWSGPQFEKWSGPQFEK |
| | (SEQ ID NO: 27) | (SEQ ID NO: 24) |
| 76 | DYKDHDGDYKDDDDK | DLYDHDGDLYDHDIDLYDDDDK |
| | (SEQ ID NO: 27) | (SEQ ID NO: 7) |
| 77 | DYKDHDGDYKDHDIDYKDDDDK | DLYDDDD |
| | (SEQ ID NO: 6) | (SEQ ID NO: 2)K |
| 78 | DYKDHDGDYKDHDIDYKDDDDK | YPYDVPDYA |
| | (SEQ ID NO: 6) | (SEQ ID NO: 3) |
| 79 | DYKDHDGDYKDHDIDYKDDDDK | SWKDASGWS |
| | (SEQ ID NO: 6) | (SEQ ID NO: 4) |
| 80 | DYKDHDGDYKDHDIDYKDDDDK | EQKLISEEDL |
| | (SEQ ID NO: 6) | (SQ ID NO: 5) |
| 81 | DYKDHDGDYKDHDIDYKDDDDK | AWRHPQFGG |
| | (SEQ ID NO: 6) | (SEQ ID NO 19) |
| 82 | DYKDHDGDYKDHDIDYKDDDDK | WSGPQFEK |
| | (SEQ ID NO: 6) | (SEQ ID NO: 20) |
| 83 | DYKDHDGDYKDHDIDYKDDDDK | DLYDDDDKDLYDDDDK |
| | (SEQ ID NO: 6) | 9SEQ ID NO: 30) |
| 84 | DYKDHDGDYKDHDIDYKDDDDK | YPYDVPDYAYPYDVPDYA |
| | (SEQ ID NO: 6) | 9SEQ ID NO: 8) |
| 85 | DYKDHDGDYKDHDIDYKDDDDK | SWKDASGWSSWKDASGWS |
| | (SEQ ID NO: 6) | (SEQ ID NO: 10) |
| 86 | DYKDHDGDYKDHDIDYKDDDDK | EQKLISEEDLEQKLISEEDL |
| | (SEQ ID NO: 6) | (SEQ ID NO: 36) |
| 87 | DYKDHDGDYKDHDIDYKDDDDK | AWRHPQFGGAWRHPQFGG |
| | (SEQ ID NO: 6) | (SEQ ID NO: 21) |
| 88 | DYKDHDGDYKDHDIDYKDDDDK | WSGPQFEKWSGPQFEK |
| | (SEQ ID NO: 6) | (SEQ ID NO: 23) |
| 89 | DYKDHDGDYKDHDIDYKDDDDK | DLYDDDDKDLYDDDDKDLYDDDDK |
| | (SEQ ID NO: 6) | (SEQ ID NO: 31) |
| 90 | DYKDHDGDYKDHDIDYKDDDDK | YPYDVPDYAYPYDVPDYAYPYDVPDYA |
| | (SEQ ID NO: 6) | (SEQ ID NO: 9) |
| 91 | DYKDHDGDYKDHDIDYKDDDDK | SWKDASGWSSWKDASGWSSWKDASGWS |
| | (SEQ ID NO: 6) | (SEQ ID NO: 11) |
| 92 | DYKDHDGDYKDHDIDYKDDDDK | EQKLISEEDLEQKLISEEDLEQKLISEEDL |
| | (SEQ ID NO: 6) | (SEQ ID NO: 37) |
| 93 | DYKDHDGDYKDHDIDYKDDDDK | AWRHPQFGGAWRHPQFGGAWRHPQFGG |
| | (SEQ ID NO: 6) | (SEQ ID NO: 22) |
| 94 | DYKDHDGDYKDHDIDYKDDDDK | WSGPQFEKWSGPQFEKWSGPQFEK |
| | (SEQ ID NO: 6) | (SEQ ID NO: 24) |
| 95 | DYKDHDGDYKDHDIDYKDDDDK | DLYDHDGDLYDHDIDLYDDDDK |
| | (SEQ ID NO: 6) | (SEQ ID NO: 7) |
| 96 | DLYDDDDK | DYKDDDDK |
| | (SEQ ID NO: 2) | (SEQ ID NO: 1) |
| 97 | YPYDVPDYA | DYKDDDDK |
| | (SEQ ID NO: 3 | (SEQ ID NO: 1) |
| 98 | SWKDASGWS | DYKDDDDK |
| | (SEQ ID NO: 4) | (SEQ ID NO: 1) |
| 99 | EQKLISEEDL | DYKDDDDK |
| | (SEQ ID NO: 5) | (SEQ ID NO: 1) |
| 100 | AWRHPQFGG | DYKDDDDK |
| | (SEQ ID NO: 19) | (SEQ ID NO: 1) |
| 101 | WSGPQFEK | DYKDDDDK |
| | (SEQ ID NO: 20) | (SEQ ID NO: 1) |
| 102 | DLYDDDDKDLYDDDDK | DYKDDDDK |
| | (SEQ ID NO: 30) | (SEQ ID NO: 1) |
| 103 | YPYDVPDYAYPYDVPDYA | DYKDDDDK |
| | (SEQ ID NO: 8) | (SEQ ID NO: 1) |
| 104 | SWKDASGWSSWKDASGWS | DYKDDDDK |
| | (SEQ ID NO: 10) | (SEQ ID NO: 1) |
| 105 | EQKLISEEDLEQKLISEEDL | DYKDDDDK |
| | (SEQ ID NO: 36) | (SEQ ID NO: 1) |
| 106 | AWRHPQFGGAWRHPQFGG | DYKDDDDK |
| | (SEQ ID NO: 21) | (SEQ ID NO: 1) |
| 107 | WSGPQFEKWSGPQFEK | DYKDDDDK |
| | (SEQ ID NO: 23) | (SEQ ID NO: 1) |
| 108 | DLYDDDDKDLYDDDDKDLYDDDDK | DYKDDDDK |
| | (SEQ ID NO: 31) | (SEQ ID NO: 1) |
| 109 | YPYDVPDYAYPYDVPDYAYPYDVPDYA | DYKDDDDK |
| | (SEQ ID NO: 9) | (SEQ ID NO: 1) |
| 110 | SWKDASGWSSWKDASGWSSWKDASGWS | DYKDDDDK |
| | (SEQ ID NO: 11) | (SEQ ID NO: 1) |
| 111 | QKLISEEDLEQKLISEEDLEQKLISEEDL | DYKDDDDK |
| | (SEQ ID NO: 37) | (SEQ ID NO: 1) |
| 112 | AWRHPQFGGAWRHPQFGGAWRHPQFGG | DYKDDDDK |
| | (SEQ ID NO: 22) | (SEQ ID NO: 1) |
| 113 | WSGPQFEKWSGPQFEKWSGPQFEK | DYKDDDDK |
| | (SEQ ID NO: 24) | (SEQ ID NO: 1) |
| 114 | DLYDHDGDLYDHDIDLYDDDDK | DYKDDDDK |
| | (SEQ ID NO: 7) | (SEQ ID NO: 1) |
| 115 | DLYDDDDK | DYKDDDDKDYKDDDDK |
| | (SEQ ID NO: 2) | (SEQ ID NO: 25) |
| 116 | YPYDVPDYA | DYKDDDDKDYKDDDDK |
| | (SEQ ID NO: 3) | (SEQ ID NO: 25) |
| 117 | SWKDASGWS | DYKDDDDKDYKDDDDK |
| | (SEQ ID NO: 4) | (SEQ ID NO: 25) |
| 118 | EQKLISEEDL | DYKDDDDKDYKDDDDK |
| | (SEQ ID NO: 5) | (SEQ ID NO: 25) |
| 119 | AWRHPQFGG | DYKDDDDKDYKDDDDK |
| | (SEQ ID NO 19) | (SEQ ID NO: 25) |
| 120 | WSGPQFEK | DYKDDDDKDYKDDDDK |
| | (SEQ ID NO: 20) | (SEQ ID NO: 25) |
| 121 | DLYDDDDKDLYDDDDK | DYKDDDDKDYKDDDDK |
| | (SEQ ID NO: 30) | (SEQ ID NO: 25) |
| 122 | YPYDVPDYAYPYDVPDYA | DYKDDDDKDYKDDDDK |
| | (SEQ ID NO: 8) | (SEQ ID NO: 25) |
| 123 | SWKDASGWSSWKDASGWS | DYKDDDDKDYKDDDDK |
| | (SEQ ID NO: 10) | (SEQ ID NO: 25) |
| 124 | EQKLISEEDLEQKLISEEDL | DYKDDDDKDYKDDDDK |
| | (SEQ ID NO: 36) | (SEQ ID NO: 25) |
| 125 | AWRHPQFGGAWRHPQFGG | DYKDDDDKDYKDDDDK |
| | (SEQ ID NO: 21) | (SEQ ID NO: 25) |
| 126 | WSGPQFEKWSGPQFEK | DYKDDDDKDYKDDDDK |
| | (SEQ ID NO: 23) | (SEQ ID NO: 25) |
| 127 | DLYDDDDKDLYDDDDKDLYDDDDK | DYKDDDDKDYKDDDDK |
| | (SEQ ID NO: 31) | (SEQ ID NO: 25) |
| 128 | YPYDVPDYAYPYDVPDYAYPYDVPDYA | DYKDDDDKDYKDDDDK |
| | (SEQ ID NO: 9) | (SEQ ID NO: 25) |
| 129 | SWKDASGWSSWKDASGWSSWKDASGWS | DYKDDDDKDYKDDDDK |
| | (SEQ ID NO: 11) | (SEQ ID NO: 25) |
| 130 | EQKLISEEDLEQKLISEEDLEQKLISEEDL | DYKDDDDKDYKDDDDK |
| | (SEQ ID NO: 37) | (SEQ ID NO: 25) |
| 131 | AWRHPQFGGAWRHPQFGGAWRHPQFGG | DYKDDDDKDYKDDDDK |
| | (SEQ ID NO: 22) | (SEQ ID NO: 25) |
| 132 | WSGPQFEKWSGPQFEKWSGPQFEK | DYKDDDDKDYKDDDDK |
| | (SEQ ID NO: 24) | (SEQ ID NO: 25) |
| 133 | DLYDHDGDLYDHDIDLYDDDDK | DYKDDDDKDYKDDDDK |
| | (SEQ ID NO: 7) | (SEQ ID NO: 25) |
| 134 | DLYDDDDK | DYKDDDDKDYKDDDDKDYKDDDDK |
| | (SEQ ID NO: 2) | (SEQ ID NO: 26) |
| 135 | YPYDVPDYA | DYKDDDDKDYKDDDDKDYKDDDDK |
| | (SEQ ID NO: 3) | (SEQ ID NO: 26) |
| 136 | SWKDASGWS | DYKDDDDKDYKDDDDKDYKDDDDK |
| | (SEQ ID NO: 4) | (SEQ ID NO: 26) |
| 137 | EQKLISEEDL | DYKDDDDKDYKDDDDKDYKDDDDK |
| | (SEQ ID NO: 5) | (SEQ ID NO: 26) |
| 138 | AWRHPQFGG | DYKDDDDKDYKDDDDKDYKDDDDK |
| | (SEQ ID NO: 19) | (SEQ ID NO: 26) |
| 139 | WSGPQFEK | DYKDDDDKDYKDDDDKDYKDDDDK |
| | (SEQ ID NO: 20) | (SEQ ID NO: 26) |
| 140 | DLYDDDDKDLYDDDDK | DYKDDDDKDYKDDDDKDYKDDDDK |
| | (SEQ ID NO: 30) | (SEQ ID NO: 26) |
| 141 | YPYDVPDYAYPYDVPDYA | DYKDDDDKDYKDDDDKDYKDDDDK |
| | (SEQ ID NO: 8) | (SEQ ID NO: 26) |
| 142 | SWKDASGWSSWKDASGWS | DYKDDDDKDYKDDDDKDYKDDDDK |
| | (SEQ ID NO: 10) | (SEQ ID NO: 26) |
| 143 | EQKLISEEDLEQKLISEEDL | DYKDDDDKDYKDDDDKDYKDDDDK |
| | (SEQ ID NO: 36) | (SEQ ID NO: 26) |
| 144 | AWRHPQFGGAWRHPQFGG | DYKDDDDKDYKDDDDKDYKDDDDK |
| | (SEQ ID NO: 21) | (SEQ ID NO: 26) |
| 145 | WSGPQFEKWSGPQFEK | DYKDDDDKDYKDDDDKDYKDDDDK |
| | (SEQ ID NO: 23) | (SEQ ID NO: 26) |
| 146 | DLYDDDDKDLYDDDDKDLYDDDD | DYKDDDDKDYKDDDDKDYKDDDDK |
| | (SEQ ID NO: 31) | (SEQ ID NO: 26) |
| 147 | YPYDVPDYAYPYDVPDYAYPYDVPDYA | DYKDDDDKDYKDDDDKDYKDDDDK |
| | (SEQ ID NO: 9) | (SEQ ID NO: 26) |
| 148 | SWKDASGWSSWKDASGWSSWKDASGWS | DYKDDDDKDYKDDDDKDYKDDDDK |
| | (SEQ ID NO: 11) | (SEQ ID NO: 26) |
| 149 | EQKLISEEDLEQKLISEEDLEQKLISEEDL | DYKDDDDKDYKDDDDKDYKDDDDK |
| | (SEQ ID NO: 37) | (SEQ ID NO: 26) |
| 150 | AWRHPQFGGAWRHPQFGGAWRHPQFGG | DYKDDDDKDYKDDDDKDYKDDDDK |
| | (SEQ ID NO: 22) | (SEQ ID NO: 26) |
| 151 | WSGPQFEKWSGPQFEKWSGPQF | DYKDDDDKDYKDDDDKDYKDDDDK |
| | (SEQ ID NO: 24) | (SEQ ID NO: 26) |
| 152 | DLYDHDGDLYDHDIDLYDDDDK | DYKDDDDKDYKDDDDKDYKDDDDK |
| | (SEQ ID NO: 7) | (SEQ ID NO: 26) |
| 153 | DLYDDDDK | DYKDHDGDYKDDDDK |
| | (SEQ ID NO: 2) | (SEQ ID NO: 27) |
| 154 | YPYDVPDYA | DYKDHDGDYKDDDDK |
| | (SEQ ID NO: 3) | (SEQ ID NO: 27) |
| 155 | SWKDASGWS | DYKDHDGDYKDDDDK |
| | (SEQ ID NO: 4) | (SEQ ID NO: 27) |
| 156 | EQKLISEEDL | DYKDHDGDYKDDDDK |
| | (SEQ ID NO: 5) | (SEQ ID NO: 27) |
| 157 | AWRHPQFGG | DYKDHDGDYKDDDDK |
| | (SEQ ID NO: 19) | (SEQ ID NO: 27) |
| 158 | WSGPQFEK | DYKDHDGDYKDDDDK |
| | (SEQ ID NO: 20) | (SEQ ID NO: 27) |
| 159 | DLYDDDDKDLYDDDDK | DYKDHDGDYKDDDDK |
| | (SEQ ID NO: 30) | (SEQ ID NO: 27) |
| 160 | YPYDVPDYAYPYDVPDYA | DYKDHDGDYKDDDDK |
| | (SEQ ID NO: 8) | (SEQ ID NO: 27) |
| 161 | SWKDASGWSSWKDASGWS | DYKDHDGDYKDDDDK |
| | (SEQ ID NO: 10) | (SEQ ID NO: 27) |
| 162 | EQKLISEEDLEQKLISEEDL | DYKDHDGDYKDDDDK |
| | (SEQ ID NO: 36) | (SEQ ID NO: 27) |
| 163 | AWRHPQFGGAWRHPQFGG | DYKDHDGDYKDDDDK |
| | (SEQ ID NO: 21) | (SEQ ID NO: 27) |
| 164 | WSGPQFEKWSGPQFEK | DYKDHDGDYKDDDDK |
| | (SEQ ID NO: 23) | (SEQ ID NO: 27) |
| 165 | DLYDDDDKDLYDDDDKDLYDDDDK | DYKDHDGDYKDDDDK |
| | (SEQ ID NO: 31) | (SEQ ID NO: 27) |
| 166 | YPYDVPDYAYPYDVPDYAYPYDVPDYA | DYKDHDGDYKDDDDK |
| | (SEQ ID NO: 9) | (SEQ ID NO: 27) |
| 167 | SWKDASGWSSWKDASGWSSWKDASGWS | DYKDHDGDYKDDDDK |
| | (SEQ ID NO: 11) | (SEQ ID NO: 27) |
| 168 | EQKLISEEDLEQKLISEEDLEQKLISEEDL | DYKDHDGDYKDDDDK |
| | (SEQ ID NO: 37) | (SEQ ID NO: 27) |
| 169 | AWRHPQFGGAWRHPQFGGAWR | DYKDHDGDYKDDDDK |
| | (SEQ ID NO: 22) | (SEQ ID NO: 27) |
| 170 | WSGPQFEKWSGPQFEKWSGPQFEK | DYKDHDGDYKDDDDK |
| | (SEQ ID NO: 24) | (SEQ ID NO: 27) |
| 171 | DLYDHDGDLYDHDIDLYDDDDK | DYKDHDGDYKDDDDK |
| | (SEQ ID NO: 7) | (SEQ ID NO: 27) |
| 172 | DLYDDDDK | DYKDHDGDYKDHDIDYKDDDDK |
| | (SEQ ID NO: 2) | (SEQ ID NO: 6) |
| 173 | YPYDVPDYA | DYKDHDGDYKDHDIDYKDDDDK |
| | (SEQ ID NO: 3) | (SEQ ID NO: 6) |
| 174 | SWKDASGWS | DYKDHDGDYKDHDIDYKDDDDK |
| | (SEQ ID NO: 4) | (SEQ ID NO: 6) |
| 175 | EQKLISEEDL | DYKDHDGDYKDHDIDYKDDDDK |
| | (SEQ ID NO: 5) | (SEQ ID NO: 6) |
| 176 | AWRHPQFGG | DYKDHDGDYKDHDIDYKDDDDK |
| | (SEQ ID NO: 19) | (SEQ ID NO: 6) |
| 177 | WSGPQFEK | DYKDHDGDYKDHDIDYKDDDDK |
| | (SEQ ID NO: 20) | (SEQ ID NO: 6) |
| 178 | DLYDDDDKDLYDDDDK | DYKDHDGDYKDHDIDYKDDDDK |
| | (SEQ ID NO: 30) | (SEQ ID NO: 6) |
| 179 | YPYDVPDYAYPYDVPDYA | DYKDHDGDYKDHDIDYKDDDDK |
| | (SEQ ID NO: 8) | (SEQ ID NO: 6) |
| 180 | SWKDASGWSSWKDASGWS | DYKDHDGDYKDHDIDYKDDDDK |
| | (SEQ ID NO: 10) | (SEQ ID NO: 6) |
| 181 | EQKLISEEDLEQKLISEEDL | DYKDHDGDYKDHDIDYKDDDDK |
| | (SEQ ID NO: 36) | (SEQ ID NO: 6) |
| 182 | AWRHPQFGGAWRHPQFGG | DYKDHDGDYKDHDIDYKDDDDK |
| | (SEQ ID NO: 21) | (SEQ ID NO: 6) |
| 183 | WSGPQFEKWSGPQFEK | DYKDHDGDYKDHDIDYKDDDDK |
| | (SEQ ID NO: 23) | (SEQ ID NO: 6) |
| 184 | DLYDDDDKDLYDDDDKDLYDDDDK | DYKDHDGDYKDHDIDYKDDDDK |
| | (SEQ ID NO: 31) | (SEQ ID NO: 6) |
| 185 | YPYDVPDYAYPYDVPDYAYPYDVPDYA | DYKDHDGDYKDHDIDYKDDDDK |
| | (SEQ ID NO: 9) | (SEQ ID NO: 6) |
| 186 | SWKDASGWSSWKDASGWSSWKDASGWS | DYKDHDGDYKDHDIDYKDDDDK |
| | (SEQ ID NO: 11) | (SEQ ID NO: 6) |
| 187 | EQKLISEEDLEQKLISEEDLEQKLISEEDL | DYKDHDGDYKDHDIDYKDDDDK |
| | (SEQ ID NO: 37) | (SEQ ID NO: 6) |
| 188 | AWRHPQFGGAWRHPQFGGAWRHPQFGG | DYKDHDGDYKDHDIDYKDDDDK |
| | (SEQ ID NO: 22) | (SEQ ID NO: 6) |
| 189 | WSGPQFEKWSGPQFEKWSGPQFEK | DYKDHDGDYKDHDIDYKDDDDK |
| | (SEQ ID NO: 24) | (SEQ ID NO: 6) |
| 190 | DLYDHDGDLYDHDIDLYDDDDK | DYKDHDGDYKDHDIDYKDDDDK |
| | (SEQ ID NO: 7) | (SEQ ID NO: 6) |

The antigenic domain may also comprise a sequence containing one or more antigenic determinants, wherein one of the antigenic determinants contains a linking sequence (this copy of the antigenic determinant sometimes being referred to simply as a linking sequence) that is cleavable by use of a sequence specific protease (as discussed in greater detail below). For example, in one embodiment of the present invention, the recombinant polypeptide, protein, or protein fragment comprises one or more antigenic domains each comprising one or more copies of an antigenic determinant generally corresponding to the FLAG® (Sigma-Aldrich, St. Louis, Mo.) peptide sequence wherein one of the copies of the antigenic determinant contains a single enterokinase cleavage site. Such antigenic domains generally correspond to the sequence:
X²⁰—(X¹—Y—K—X²—X³-D-X⁴)ₙ—X⁵—(X¹—Y—K—X⁷—X⁸-D-X⁹—K)—X²¹
where:
D, Y and K are their representative amino acids, as known in the art;
X²⁰ and X²¹ are independently a hydrogen or a covalent bond;
each X¹ and X⁴ is independently a covalent bond or at least one amino acid residue, if other than a covalent bond, preferably at least one amino acid residue selected from the group consisting of aromatic amino acid residues and hydrophilic amino acid residues, more preferably at least one hydrophilic amino acid residue, and still more preferably at least one an aspartate residue;
each X², X³, X⁷ and X⁸ is independently an amino acid residue, preferably an amino acid residue selected from the group consisting of aromatic amino acid residues and hydrophilic amino acid residues, more preferably a hydrophilic amino acid residue, and still more preferably an aspartate residue;
X⁵ is a covalent bond or a spacer domain comprising at least one amino acid, if other than a covalent bond, preferably a histidine residue, a glycine residue or a combination of multiple or alternating histidine residues, said combination comprising His-Gly-His, or -(His-X)ₘ—, wherein m is 1 to 6 and X is selected from the group consisting of Ala, Arg, Asn, Asp, Cys, Gln, Glu, Gly, H is, lie, Leu, Lys, Phe, Pro, Ser, Thr, Trp, Tyr and Val;
X⁹ is a covalent bond or D; and
n is 0, 1 or 2.

In this embodiment, the amino acid sequence X²⁰—(X¹—Y—K—X²—X³-D-X⁴)ₙ comprises or may comprise antigenic determinants —X¹—Y—K—X²—X³-D-X⁴ joined in tandem which are joined to another copy of the antigenic determinant containing a linking sequence (X¹—Y—K—X⁷—X⁸-D-X⁹—K). The antigenic determinants may be immediately adjacent to each other when n is at least one and X⁴ is a covalent bond; optionally, X⁴ may be a spacer domain interposed between the multiple copies of antigenic determinants. The linking sequence contains a single enterokinase cleavable site which is represented by the sequence —X⁷—X⁸-D-X⁹—K, where X⁷ and X⁸ may be an amino acid residue or a covalent bond and X⁹ is a covalent bond or an aspartate residue. In one embodiment, each X⁷, X⁸ and X⁹ is independently an aspartate residue thus resulting in the enterokinase cleavable site DDDDK (SEQ ID NO: 12) which is preferably located immediately adjacent to the amino terminus of the target peptide. When n is at least one and X⁵ is a covalent bond, the multiple copies of antigenic determinants may be immediately adjacent to the linking sequence; optionally, X⁵ may be a spacer domain interposed between the linking sequence and the antigenic determinants. When each X⁴ and X⁵ is independently a spacer domain, it is preferred that the amino acid residue(s) of each X⁴ and X⁵ impart one or more desired properties to the antigenic determinant; for example, the amino acids of the spacer domain may be selected to impart a desired folding to the identification polypeptide thereby increasing accessibility to the antibody. In another embodiment, the amino acids of the spacer domain X⁴ and X⁵ may be selected to impart a desired affinity characteristic such as a combination of multiple or alternating histidine residues capable of chelating to an immobilized metal ion on a resin or other matrix. Furthermore, these desired properties may be designed into other areas of the identification polypeptide; for example, the amino acids represented by X² and X³ may be selected to impart a desired peptide folding or a desired affinity characteristic for use in affinity purification.

In another embodiment, one or both of the antigenic domains may comprise a linking sequence containing a single enterokinase or other cleavage site, or generally correspond to the sequence:
X²⁰-(D-Y—K—X²—X³-D)ₙ-X⁵-(D-Y—K—X⁷—X⁸-D-X⁹—K)—X²¹
where:
D, Y, K are their representative amino acids;
X²⁰ and X²¹ are independently a hydrogen or a covalent bond;
each X², X³, X⁷ and X⁸ is independently an amino acid residue, preferably an amino acid residue selected from the group consisting of aromatic amino acid residues and hydrophilic amino acid residues, more preferably a hydrophilic amino acid residue, and still more preferably an aspartate residue;
X⁵ is a covalent bond or a spacer domain comprising at least one amino acid, if other than a covalent bond, preferably a histidine residue, a glycine residue or a combination of multiple or alternating histidine residues, said combination comprising His-Gly-His, or -(His-X)ₘ—, wherein m is 1 to 6 and X is selected from the group consisting of Ala, Arg, Asn, Asp, Cys, Gln, Glu, Gly, H is, lie, Leu, Lys, Met, Phe, Pro, Ser, Thr, Trp, Tyr and Val;
X⁹ is a covalent bond or an aspartate residue; and
n is at least 2.

In this embodiment, the amino acid sequence X²⁰-(D-Y—K—X²—X³-D)ₙ represents multiple copies of the antigenic determinant D-Y—K—X²—X³-D in tandem which are joined to a linking sequence (D-Y—K—X⁷—X⁸-D-X⁹—K). In this embodiment, one antigenic determinant is immediately adjacent to another antigenic determinant, i.e., no intervening spacer domains, and the multiple copies of the antigenic determinant are immediately adjacent to the linking sequence when X⁵ is a covalent bond. The linking sequence contains a single enterokinase cleavable site which is represented by the sequence —X⁷—X⁸-D-X⁹—K, where X⁷ and X⁸ may be a covalent bond or an amino acid residue, preferably an aspartate residue, and X⁹ is a covalent bond or an aspartate residue. In one embodiment, each X⁷, X⁸ and X⁹ is independently an aspartate residue thus resulting in the enterokinase cleavable site DDDDK (SEQ ID NO: 12) which is preferably adjacent to the amino terminus of the target peptide. Optionally, the multiple copies of the antigenic domain are joined to the linking sequence by a spacer X⁵ when X⁵ is at least one amino acid residue. When X⁵ is a spacer domain, it is preferred that the amino acid residue(s) of X⁵ impart one or more desired properties to the recombinant polypeptide, protein, or protein fragment; for example, the amino acids of the spacer domain may be selected to impart a desired folding to the recombinant polypeptide, protein, or protein fragment thereby increasing accessibility to the antibody. In another embodiment, the amino acids of the spacer domain may be selected to impart a desired affinity characteristic such as a combination of multiple or alternating histidine residues capable of chelating to an immobilized metal ion on a resin or other matrix. Furthermore, these desired properties may be designed into other areas of the spacer; for example, the amino acids represented by X² and X³ may be selected to impart a desired peptide folding or a desired affinity characteristic for use in affinity purification.

When the affinity polypeptide is located at the amino terminus of the target polypeptide, protein, or protein fragment, it is often desirable to design the amino acid sequence such that an initiator methionine is present. Accordingly, in one embodiment of the present invention, the recombinant polypeptide, protein or protein fragment comprises multiple copies of an antigenic determinant, a linking sequence containing a single enterokinase cleavage site and generally corresponds to the sequence:
X²⁰—X¹⁰-(D-Y—K—X²—X³-D)ₙ-X⁵-(D-Y—K—X⁷—X⁸-D-X⁹—K)—X²¹
where:
D, Y, and K are their representative amino acids;
X²⁰ and X²¹ are independently a hydrogen or a covalent bond;
X¹⁰ is a covalent bond or an amino acid, if other than a covalent bond, preferably a methionine residue;
each X², X³, X⁷ and X⁸ is independently an amino acid residue, preferably an amino acid residue selected from the group consisting of aromatic amino acid residues and hydrophilic amino acid residues, more preferably a hydrophilic amino acid residue, and still more preferably an aspartate residue;
X⁵ is a covalent bond or a spacer domain comprising at least one amino acid, if other than a bond, preferably a histidine residue, a glycine residue or a combination of multiple or alternating histidine residues, said combination comprising His-Gly-His, or -(His-X)ₘ—, wherein m is 1 to 6 and X is selected from the group consisting of Ala, Arg, Asn, Asp, Cys, Gln, Glu, Gly, H is, lie, Leu, Lys, Met, Phe, Pro, Ser, Thr, Trp, Tyr, and Val;
X⁹ is a covalent bond or an aspartate residue; and
n is at least 2.

In this embodiment, the amino acid sequence X²⁰-(D-Y—K—X²—X³-D)ₙ represents the multiple copies of the antigenic determinant D-Y—K—X²—X³-D in tandem which is flanked by a linking sequence (D-Y—K—X⁷—X⁸-D-X⁹—K) and an initiator amino acid X¹⁰, preferably methionine. The antigenic determinant D-Y—K—X²—X³-D with an initiator methionine is recognized by the Monoclonal ANTI-FLAG® M5 antibody (Sigma-Aldrich, St. Louis, Mo.). In this embodiment, one antigenic determinant is immediately adjacent to another antigenic determinant, i.e., no intervening spacer domains, and the multiple copies of the antigenic determinant are immediately adjacent to the linking sequence when X⁵ is a covalent bond. The linking sequence contains an enterokinase cleavable site which is represented by the amino acid sequence —X⁷—X⁸-D-X⁹—K, where X⁷ and X⁸ may be a covalent bond or an amino acid residue, preferably an aspartate residue, and X⁹ is a covalent bond or an aspartate residue. In one embodiment, each X⁷, X⁸ and X⁹ is independently an aspartate residue thus resulting in the enterokinase cleavable site DDDDK (SEQ ID NO: 12) which is preferably adjacent to the amino terminus of the target peptide. Optionally, the multiple copies of the antigenic determinant are joined to the linking sequence by a spacer domain X⁵ when X⁵ is at least one amino acid residue. When X⁵ is a spacer domain, it is preferred that the amino acid residue(s) of X⁵ impart one or more desired properties to the affinity polypeptide; for example, the amino acids of the spacer domain may be selected to impart a desired folding to the recombinant polypeptide, protein, or protein fragment thereby increasing accessibility to the antibody. In another embodiment, the amino acids of the spacer domain may be selected to impart a desired affinity characteristic such as a combination of multiple or alternating histidine residues capable of chelating to an immobilized metal ion on a resin or other matrix. Furthermore, these desired properties may be designed into other areas of the affinity polypeptide; for example, the amino acids represented by X² and X³ may be selected to impart a desired peptide folding or a desired affinity characteristic for use in affinity purification. In a particular embodiment, the amino acid sequence is M-D-Y—K-D-H-D-G-D-Y—K-D-H-D-I-D-Y—K-D-D-D-D-K—X²¹ (SEQ ID NO: 13), wherein X²¹ is a hydrogen or a covalent bond. In a particularly preferred embodiment, X²¹ is a hydrogen.

In another embodiment of the present invention, the recombinant polypeptide, protein, or protein fragment comprises one or more copies of an antigenic determinant, a linking sequence containing a single enterokinase cleavable site and generally corresponds to the sequence:
X²⁰-(D-X¹¹—Y—X¹²—X¹³)ₙ—X¹⁴-(D-X¹¹—Y—X¹²—X¹³-D-X¹⁵—K)—X²¹
where:
D, Y and K are their representative amino acids;
X²⁰ and X²¹ are independently a hydrogen or a covalent bond;
each X¹¹ is a covalent bond or an amino acid, preferably Leu;
each X¹² is an amino acid, preferably selected from the group consisting of aromatic amino acid residues and hydrophilic amino acid residues, more preferably a hydrophilic amino acid residue, and still more preferably an aspartate residue;
each X¹³ is a covalent bond or at least one amino acid, if other than a covalent bond, preferably selected from the group consisting of aromatic amino acid residues and hydrophilic amino acid residues, more preferably a hydrophilic amino acid residue, and still more preferably an aspartate residue;
X¹⁴ is a covalent bond or a spacer domain comprising at least one amino acid, if other than a covalent bond, preferably a histidine residue, a glycine residue or a combination of multiple or alternating histidine residues, said combination comprising His-Gly-His, or -(His-X)ₘ—, wherein m is 1 to 6 and X is selected from the group consisting of Ala, Arg, Asn, Asp, Cys, Gln, Glu, Gly, H is, lie, Leu, Lys, Met, Phe, Pro, Ser, Thr, Trp, Tyr and Val;
X¹⁵ is a covalent bond or an aspartate residue; and
n is at least 0 or at least 1.

In this embodiment, when n is at least 2, the amino acid sequence X²⁰-(D-X¹¹—Y—X¹²—X¹³)ₙ constitutes multiple copies of the antigenic determinant D-X¹¹—Y—X¹²—X¹³ in tandem which are joined to a linking sequence (D-X¹¹—Y—X¹²—X¹³-D-X¹⁵—K). Additionally, one antigenic determinant may be immediately adjacent to another antigenic determinant, i.e., no intervening spacer domains, and the multiple copies of the antigenic domain may be immediately adjacent to the linking sequence when X¹⁴ is a covalent bond. The linking sequence contains a single enterokinase cleavable site which is represented by the sequence —X¹²—X¹³-D-X¹⁵—K where X¹² and X¹³ may be a covalent bond or an amino acid residue, preferably an aspartate residue, and X¹⁵ is a covalent bond or an aspartate residue. In one embodiment, each X¹², X¹³ and X¹⁵ is independently an aspartate residue thus resulting in the enterokinase cleavable site DDDDK (SEQ ID NO: 12) which is preferably adjacent to the amino terminus of the target peptide. Optionally, when n is at least two, the multiple copies of the antigenic determinant are joined to the linking sequence by a spacer X¹⁴ when X¹⁴ is at least one amino acid residue. When X¹⁴ is a spacer domain, it is preferred that the amino acid residue(s) of X¹⁴ impart one or more desired properties to the recombinant polypeptide, protein or protein fragment; for example, the amino acids of the spacer domain may be selected to impart a desired folding to the recombinant polypeptide, protein or protein fragment thereby increasing accessibility to the antibody. In another embodiment, the amino acids of the spacer domain X¹⁴ may be selected to impart a desired affinity characteristic such as a combination of multiple or alternating histidine residues capable of chelating to an immobilized metal ion on a resin or other matrix.

When the affinity polypeptide is located at the amino terminus of the target polypeptide, protein or protein fragment, it is often desirable to design the amino acid sequence such that an initiator methionine is present. In another embodiment of the present invention, the recombinant polypeptide, protein, or protein fragment comprises one or more copies of an antigenic determinant, a linking sequence containing a single enterokinase cleavable site and generally corresponds to the sequence:
X²⁰—X¹⁰-(D-X¹¹—Y—X¹²—X¹³)ₙ—X¹⁴-(D-X¹¹—Y—X¹²—X¹³-D-X¹⁵—K)—X²¹
where:
D, Y and K are their representative amino acids;
X²⁰ and X²¹ are independently a hydrogen or a covalent bond;
X¹⁰ is a covalent bond or an amino acid, if other than a covalent bond, preferably a methionine residue;
each X¹¹ is a covalent bond or an amino acid, preferably Leu;
each X¹² is an amino acid, preferably selected from the group consisting of aromatic amino acid residues and hydrophilic amino acid residues, more preferably a hydrophilic amino acid residue, and still more preferably an aspartate residue;
each X¹³ is a covalent bond or at least one amino acid, if other than a covalent bond, preferably selected from the group consisting of aromatic amino acid residues and hydrophilic amino acid residues, more preferably a hydrophilic amino acid residue, and still more preferably an aspartate residue;
X¹⁴ is a covalent bond or a spacer domain comprising at least one amino acid, if other than a covalent bond, preferably a histidine residue, a glycine residue or a combination of multiple or alternating histidine residues, said combination comprising His-Gly-His, or -(His-X)ₘ—, wherein m is 1 to 6 and X is selected from the group consisting of Ala, Arg, Asn, Asp, Cys, Gln, Glu, Gly, H is, Ile, Leu, Lys, Met, Phe, Pro, Ser, Thr, Trp, Tyr and Val;
X¹⁵ is a covalent bond or an aspartate residue; and
n is at least 0 or at least 1.

In this embodiment, when n is at least 2, the amino acid sequence X²⁰-(D-X¹¹—Y—X¹²—X¹³)ₙ constitutes multiple copies of the antigenic determinant D-X¹¹—Y—X¹²—X¹³ in tandem which are joined to a linking sequence (D-X¹¹—Y—X¹²—X¹³-D-X¹⁵—K). Additionally, one antigenic determinant may be immediately adjacent to another antigenic determinant, i.e., no intervening spacer domains, and the multiple copies of the antigenic domain may be immediately adjacent to the linking sequence when X¹⁴ is a covalent bond. The linking sequence contains a single enterokinase cleavable site which is represented by the sequence —X¹²—X¹³-D-X¹⁵—K where X¹² and X¹³ may be a covalent bond or an amino acid residue, preferably an aspartate residue, and X¹⁵ is a covalent bond or an aspartate residue. In one embodiment, each X¹², X¹³ and X¹⁵ is independently an aspartate residue thus resulting in the enterokinase cleavable site DDDDK (SEQ ID NO: 12) which is preferably adjacent to the amino terminus of the target peptide. Optionally, when n is at least two, the multiple copies of the antigenic determinant are joined to the linking sequence by a spacer X¹⁴ when X¹⁴ is at least one amino acid residue. When X¹⁴ is a spacer domain, it is preferred that the amino acid residue(s) of X¹⁴ impart one or more desired properties to the recombinant polypeptide, protein or protein fragment; for example, the amino acids of the spacer domain may be selected to impart a desired folding to the recombinant polypeptide, protein or protein fragment thereby increasing accessibility to the antibody. In another embodiment, the amino acids of the spacer domain X¹⁴ may be selected to impart a desired affinity characteristic such as a combination of multiple or alternating histidine residues capable of chelating to an immobilized metal ion on a resin or other matrix. In a particular embodiment, the amino acid sequence is M-D-L-Y-D-H-D-G-D-L-Y-D-H-D-I-D-L-Y-D-D-D-D-K—X²¹ (SEQ ID NO: 14), wherein X²¹ is a hydrogen or a covalent bond. In a particularly preferred embodiment, X²¹ is a hydrogen.

Target Polypeptide, Protein, or Protein Fragment

The target polypeptide, protein or protein fragment may be composed of any proteinaceous substance that can be expressed in transformed host cells. Accordingly, the present invention may be beneficially employed to produce substantially any prokaryotic or eukaryotic, simple or conjugated, protein that can be expressed by a vector in a transformed host cell. For example, the target protein may be
a) an enzyme, whether oxidoreductase, transferase, hydrolase, lyase, isomerase or ligase;
b) a storage protein, such as ferritin or ovalbumin or a transport protein, such as hemoglobin, serum albumin or ceruloplasmin;
c) a protein that functions in contractile and motile systems such as actin or myosin;
d) any of a class of proteins that serve a protective or defense function, such as the blood protein fibrinogen or a binding protein, such as antibodies or immunoglobulins that bind to and thus neutralize antigens;
e) a hormone such as human Growth Hormone, somatostatin, prolactin, estrone, progesterone, melanocyte, thyrotropin, calcitonin, gonadotropin and insulin;
f) a hormone involved in the immune system, such as interleukin-1, interleukin-2, colony stimulating factor, macrophage-activating factor and interferon;
g) a toxic protein, such as ricin from castor bean or gossypin from cotton linseed;
h) a protein that serves as structural elements such as collagen, elastin, alpha-keratin, glyco-proteins, viral proteins and mucoproteins; or
i) a synthetic protein, defined generally as any sequence of amino acids not occurring in nature.

In general, the target polypeptide, protein, or protein fragment may be a constituent of the R₁, R₂, and R₃ moieties of the recombinant polypeptides, proteins or protein fragments corresponding to general formula described above.

Genes coding for the various types of protein molecules identified above may be obtained from a variety of prokaryotic or eukaryotic sources, such as plant or animal cells or bacteria cells. The genes can be isolated from the chromosome material of these cells or from plasmids of prokaryotic cells by employing standard, well-known techniques known to those skilled in the art. A variety of naturally occurring and synthesized plasmids having genes coding for many different protein molecules are not commercially available from a variety of sources. The desired DNA also can be produced from mRNA by using the enzyme reverse transcriptase. This enzyme permits the synthesis of DNA from an RNA template.

In one embodiment, R₁ may be a protein, which enhances expression and R₂ or R₃ is the target polypeptide, protein, or protein fragment. It is well known that the presence of some proteins in a cell result in expression of genes. If a chimeric protein contains an active portion of the protein, which prompts or enhances expression of the gene encoding it, greater quantities of the protein may be expressed than if it were not present.

Expression of Recombinant Proteins

The polypeptides, proteins, and protein fragments of the present invention are generally prepared and expressed as a fusion protein using conventional recombinant DNA technology. The fusion protein is thus produced by host cells transformed with the genetic information encoding the fusion protein. The host cells may secrete the fusion protein into the culture media or store it in the cells whereby the cells must be collected and disrupted in order to extract the product. As hosts, E. coli, yeast, insect cells, mammalian cells and plants are suitable. Of these two, E. coli will typically be the more preferred host for most applications. In one embodiment, the recombinant polypeptides, proteins, and protein fragments are produced in a soluble form or secreted from the host.

In general, a chimeric gene is inserted into an expression vector which allows for the expression of the desired fusion protein in a suitable transformed host. The expression vector provides the inserted chimeric gene with the necessary regulatory sequences to control expression in the suitable transformed host.

There are six elements of control expression sequence for proteins which are to be secreted from a host into the medium, while five of these elements apply to fusion proteins expressed intracellularly. These elements, in the order they appear in the gene, are: a) the promoter region; b) the 5′ untranslated region; c) signal sequence; d) the chimeric coding sequence; e) the 3′ untranslated region; and f) the transcription termination site. Fusion proteins which are not secreted do not typically contain c), the signal sequence.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, operably linked to the nucleic acid sequence to be expressed. It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

Vector DNA can be introduced into prokaryotic or eukaryotic cells via conventional transformation or transfection techniques. For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin, and methotrexate. Nucleic acid encoding a selectable marker can be introduced into a host cell on the same vector as that encoding the antigenic domain containing fusion protein or can be introduced on a separate vector. Cells stably transfected with the introduced nucleic acid can be identified by drug selection (e.g., cells that have incorporated the selectable marker gene will survive, while the other cells die). Methods and materials for preparing recombinant vectors, transforming host cells using replicating vectors, and expressing biologically active foreign polypeptides and proteins are generally well known and are described in Sambrook, et al., Molecular Cloning: a Laboratory Manual, 3rd ed (2001) Cold Spring Harbor Press.

Accordingly, another aspect of the present invention is a nucleic acid sequence encoding any of the aforementioned polypeptides, protein, or protein fragments represented by the formula R₁—Sp₁-Tag₁-Sp₂-R₂—Sp₃-Tag₂-Sp₄-R₃, wherein R₁ is hydrogen, a polypeptide, a protein, or a protein fragment; Sp₁ is a bond or a spacer comprising at least one amino acid residue; Tag₁ is an antigenic domain, said antigenic domain comprising at least one antigenic determinant selected from the group consisting of a peptide sequence having affinity for the M1 antibody, a peptide sequence having affinity for the M2 antibody, a peptide sequence having affinity for the M5 antibody, a peptide sequence having affinity for the peptide sequence DYKDDDDK (SEQ ID NO: 1), a peptide sequence having affinity for the peptide sequence DLYDDDDK (SEQ ID NO: 2), the HA epitope, c-myc, AcV5, and a peptide sequence having affinity for streptavidin or streptavidin derivatives; Sp₂ is a bond or a spacer comprising at least one amino acid residue; R₂ is a bond, a polypeptide, a protein, or a protein fragment; Sp₃ is a bond or a spacer comprising at least one amino acid; Tag₂ is an antigenic domain, said antigenic domain comprising at least one antigenic determinant selected from the group consisting of a peptide sequence having affinity for the M1 antibody, a peptide sequence having affinity for the M2 antibody, a peptide sequence having affinity for the M5 antibody, a peptide sequence having affinity for the peptide sequence DYKDDDDK (SEQ ID NO: 1), a peptide sequence having affinity for the peptide sequence DLYDDDDK (SEQ ID NO: 2), the HA epitope, c-myc, AcV5, and a peptide sequence having affinity for streptavidin or streptavidin derivatives; Sp₄ is a bond or a spacer comprising at least one amino acid; and R₃ is hydrogen, a polypeptide, a protein, or a protein fragment; wherein when Sp₂, R₂, and Sp₃ are each bonds Tag₁ and Tag₂ are not the same antigenic domain. Another aspect of the present invention is vectors comprising such nucleic acid sequences. Still another aspect of the present invention is host cells contain these expression vectors.

Spacers and Linkers and Other Optional Elements

Spacers and Linkers

In one embodiment, the recombinant polypeptide, protein or protein fragment includes a spacer (Sp₁ or Sp₂) between the metal ion-affinity polypeptide and the target polypeptide, protein or protein fragment. If present, the spacer may simply comprise one or more, e.g., three to ten amino acid residues, separating the metal ion-affinity peptide from the target polypeptide, protein or protein fragment. Alternatively, the spacer may comprise a sequence which imparts other functionality, such as a proteolytic cleavage site, a fusion protein, a secretion sequence (e.g. OmpA or OmpT for E. coli, preprotrypsin for mammalian cells, a-factor for yeast, and melittin for insect cells), a leader sequence for cellular targeting, antibody epitopes, or IRES (internal ribosomal entry sequences) sequences.

In one embodiment, the spacer is selected from among hydrophilic amino acids to increase the hydrophilic character of the recombinant polypeptide, protein or protein fragment. Alternatively, the amino acid(s) of the spacer domain may be selected to impart a desired folding to the recombinant polypeptide, protein or protein fragment thereby increasing accessability to one or more regions of the molecule. For example, the spacer domain may comprise glycine residues which results in a protein folding conformation which allows for improved accessibility to antibodies.

In another embodiment, the spacer comprises a cleavage site which consists of a unique amino acid sequence cleavable by use of a sequence-specific proteolytic agent. Such a site would enable the metal ion-affinity polypeptide to be readily cleaved from the target polypeptide, protein or protein fragment by digestion with a proteolytic agent specific for the amino acids of the cleavage site. Alternatively, the metal ion-affinity peptide may be removed from the desired protein by chemical cleavage using methods known to the art.

When present, the cleavable site may be located at the amino or carboxy terminus of the target peptide. Preferably, the cleavable site is immediately adjacent to the desired protein to enable separation of the desired protein from the metal ion-affinity peptide. This cleavable site preferably does not appear in the desired protein. In one embodiment, the cleavable site is located at the amino terminus of the desired protein. If the cleavable site is located at the amino terminus of the desired protein and if there are remaining extraneous amino acids on the desired protein after cleavage with the proteolytic agent, an endopeptidase such as trypsin, clostropain or furin may be utilized to remove these remaining amino acids, thus resulting in a highly purified desired protein. Further examples of proteolytic enzymatic agents useful for cleavage are papain, pepsin, plasmin, thrombin, enterokinase, and the like. Each affects cleavage at a particular amino acid sequence which it recognizes.

Digestion with a proteolytic agent may occur while the fusion protein is still bound to the affinity resin or alternatively, the fusion protein may be eluted from the affinity resin and then digested with the proteolytic agent in order to further purify the desired protein. Preferably, the amino acid sequence of the proteolytic cleavage site is unique, thus minimizing the possibility that the proteolytic agent will cleave the desired protein. In one embodiment, the cleavable site comprises amino acids for an enterokinase, thrombin or a Factor Xa cleavage site.

Enterokinase recognizes several sequences: Asp-Lys; Asp-Asp-Lys; Asp-Asp-Asp-Lys (SEQ ID NO: 34); and Asp-Asp-Asp-Asp-Lys (SEQ ID NO: 12). The only known natural occurrence of Asp-Asp-Asp-Asp-Lys (SEQ ID NO: 12) is in the protein trypsinogen which is a natural substrate for bovine enterokinase and some yeast proteins. As such, by interposing a fragment containing the amino acid sequence Asp-Asp-Asp-Asp-Lys (SEQ ID NO: 12) as a cleavable site between the metal ion-affinity polypeptide and the amino terminus of the target polypeptide, protein or protein fragment, the metal ion-affinity polypeptide can be liberated from the desired protein by use of bovine enterokinase with very little likelihood that this enzyme will cleave any portion of the desired protein itself.

Thrombin cleaves on the carboxy-terminal side of arginine in the following sequence: Leu-Val-Pro-Arg-Gly-X (SEQ ID NO: 15), where X is a non-acidic amino acid. Factor Xa protease (i.e., the activated form of Factor X) cleaves after the Arg in the following sequences: Ile-Glu-Gly-Arg-X (SEQ ID NO: 16), Ile-Asp-Gly-Arg-X (SEQ ID NO: 17), and Ala-Glu-Gly-Arg-X (SEQ ID NO: 18), where X is any amino acid except proline or arginine. A fusion protein comprising the 31 amino-terminal residues of the cII protein, a Factor Xa cleavage site and human -globin was shown to be cleaved by Factor Xa and generate authentic β-globin. A limitation of the Factor Xa-based fusion systems is the fact that Factor Xa has been reported to cleave at arginine residues that are not present within in the Factor Xa recognition sequence (Nagai, K, et al., Prot. Expr. and Purif., 2:372 (1991)).

While less preferred, other unique amino acid sequences for other cleavable sites may also be employed in the spacer without departing from the spirit or scope of the present invention. For instance, the spacer may be composed, at least in part, of a pair of basic amino acids, i.e., Arg, H is or Lys. This sequence is cleaved by kallikreins, a glandular enzyme. Also, the spacer may be composed, at least in part, of Arg-Gly, since it is known that the enzyme thrombin will cleave after the Arg if this residue is followed by Gly.

Metal Affinity (MAT) Tags

In addition to the multiple antigenic domains, the recombinant polypeptide, protein, or protein fragment may also comprise a metal ion-affinity peptide, sometimes referred to as a metal affinity tag (MAT or MAT tag), having the sequence His-Z₁-His-Arg-His-Z₂-His (SEQ ID NO: 35), wherein Z₁ is an amino acid residue selected from the group consisting of Ala, Arg, Asn, Asp, Gln, Glu, Ile, Lys, Phe, Pro, Ser, Thr, Trp, and Val; and Z₂ is an amino acid residue selected from the group consisting of Ala, Arg, Asn, Asp, Cys, Gln, Glu, Gly, Ile, Leu, Lys, Met, Pro, Ser, Thr, Tyr and Val. The recombinant polypeptide, protein, or protein fragment may comprise comprising multiple copies (i.e., two, three, four, five, or more) of the metal ion-affinity peptide (His-Z₁-His-Arg-His-Z₂-His (SEQ ID NO: 35)) wherein Z₁ and Z₂ are as previously defined above. In this embodiment, the additional copies of the metal affinity peptide may occur in either or both of the spacer domains (Sp₁ and Sp₂) or in either or both of the other domains (R₁ and R₂) of the recombinant polypeptides, proteins or protein fragments. Thus, for example, in one embodiment a second copy of the metal ion-affinity peptide (His-Z₁-His-Arg-His-Z₂-His) wherein Z₁ and Z₂ are as previously defined is located in one of the spacer domains (Sp₁ or Sp₂) or other domains (R₁ and R₂) of the recombinant polypeptides, proteins or protein fragments. By way of further example, in another embodiment two additional copies of the metal ion-affinity peptide (His-Z₁-His-Arg-His-Z₂-His (SEQ ID NO: 35)) wherein Z₁ and Z₂ are as previously defined are located in the spacer domains (Sp₁ or Sp₂) or other domains (R₁ and R₂) of the recombinant polypeptides, proteins or protein fragments. By way of further example, in another embodiment at least three additional copies of the metal ion-affinity peptide (His-Z₁-His-Arg-His-Z₂-His (SEQ ID NO: 35)) wherein Z₁ and Z₂ are as previously defined are located in the spacer domains (Sp₁ or Sp₂) or other domains (R₁ and R₂) of the recombinant polypeptides, proteins or protein fragments. In each of these embodiments, the multiple copies of the metal ion-affinity peptide may be separated by one or more amino acid residues (i.e., a spacer) as described herein. Alternatively, in each of these embodiments the multiple copies of the metal ion-affinity peptide may be directly linked to each other without any intervening amino acid residues.

Detection, Identification, Isolation, Capture, and Purification of Recombinant Peptides and Proteins Using the TAP Tags

The binding of antibodies to the recombinant polypeptides, proteins, or protein fragments of the invention allows for the capture of the fusion peptide or protein on a solid support. Once the antibodies bind the fusion peptide or protein, the antibodies, and therefore, the fusion peptide or protein, can be detected, for example, by simple visualization, isolated, and then purified. Methods of capturing, and subsequently detecting, isolating, and purifying, the antibody bound fusion peptide or protein is well known to those of skill in the art.

In accordance with the preferred embodiment of the invention, to capture a recombinant polypeptide, protein, or protein fragment of the invention, two affinity purification steps are performed. Each affinity step consists of a binding step in which the polypeptides, proteins, or protein fragments of the invention are bound via one of its antigenic domains to a support material which is covered with an antibody specific for that antigenic domain. Unbound substances are removed and the polypeptides, proteins, or protein fragments of the invention are recovered from the support material. This can be done, for example, in at least two ways. Conventional elution techniques such as varying the pH, the salt, or buffer concentrations and the like depending on the antigenic domain bound, can be performed. Alternatively, the protein to be purified can be released from the support material by proteolytically cleaving recombinant polypeptides, proteins, or protein fragments from the support by cleaving the antigenic domain attached thereto from the support or from the recombinant polypeptides, proteins, or protein fragments. If the cleavage step is performed, the protein can be recovered in the form of a truncated polypeptide, protein, or protein fragment if only a single antigenic domain is cleaved or if all antigenic domains affinity tags have been cleaved off, as the target polypeptide or protein if all of the antigenic domains are cleaved. Preferably, the protein to be purified is released from the support material by competition with another peptide that competitively binds either the support material or one or more of the antigenic domains of the recombinant polypeptide, protein, or protein fragment.

For example, Western blotting is a technique used to detect and isolate a captured fusion peptide or protein comprising a one or more tags or antigenic domains. Generally, small quantities of a fusion protein are electrophoresed on a polyacrylamide gel and transferred (by blotting) to a polymer sheet or membrane. The membrane is then incubated with a first antibody which binds to one or more of the antigenic domains represented by Tag₁ or Tag₂ of the fusion peptide or protein. The membrane containing the antibody-fusion peptide or protein is then incubated with a second labeled antibody specific for the first antibody. The tagged fusion peptide or protein may then be detected and visualized by known methods such as autoradiography, colorimetric and chemiluminescent detection. This process may be repeated wherein the fusion protein is further incubated with an antibody which binds to Tag₂ (or Tag₁ if initially separated by Tag₂, as the order of separation is unimportant) to further detect and isolate the fusion peptide or protein as described with respect to the first Western blotting procedure.

An example of an affinity technique is separation of antibody-bound fusion peptides or proteins marked by antibodies to Tag₁ and Tag₂ from peptides or proteins which do not express Tag₁ and Tag₂ or otherwise were not labeled by the antibodies. One example of a suitable affinity technique includes fluorescence activated cell sorting (FACS). In FACS, a secondary antibody which is tagged with a fluorescence material, such as fluorescein isothiocyanate (FITC), or rhodamine isothiocyanate (RITC), is introduced into a sample containing the antibody-bound fusion peptide or protein. The anti-Tag₁ or anti-Tag₂ antibody is attached to the fusion peptide or protein. The secondary antibody binds to the anti-Tag₁ or anti-Tag₂ antibody. Fusion peptides or proteins having the secondary antibody will fluoresce and separation may be achieved by a fluorescence activated cell sorter. FACS can be used to separate fusion peptides or proteins expressing antigenic domains such as Tag₁ and Tag₂ and bound by an anti-Tag₁ or anti-Tag₂ antibody by using, for example, an appropriate anti-mouse IgG secondary antibody tagged with a fluorescent material. These methods are well known in the art and are commonly used for other antibodies as well, as disclosed and described in Kunz et al., J. Biol. Chem., Vol. 267, pg. 9101 (1992), incorporated herein by reference. Once a first FACS separation is performed, the sorted fusion peptides or proteins can then be subjected to a second FACS separation wherein the fusion protein is further sorted based upon Tag₂ (or Tag₁ if initially separated by Tag₂, as the order of separation is unimportant) to further purify the fusion peptide or protein as described with respect to the first FACS separation.

Another affinity technique is immunoprecipitation. Immunoprecipitation employs antibodies raised against a peptide tag to precipitate a fusion peptide or protein comprising the tag from a sample containing the fusion peptide or protein. The use of immunoprecipitation is known to one skilled in the art. See, for example, Molecular Cloning, A Laboratory Manual, 2d Edition, Maniatis, T. et al. eds. (1989) Cold Spring Harbor Press and Antibodies, A Laboratory Manual, Harlow, E. and Lane, D., eds. (1988) Cold Spring Harbor Press. An example of immunoprecipitation is the use of antibodies coupled to beads. The antibodies coupled to the beads can bind directly to the Tag₁ or Tag₂. Alternatively, the secondary antibodies may be coupled to the beads, and the secondary antibodies can be specific to the anti-Tag₁ or anti-Tag₂ antibodies. A method of attaching antibodies to beads is disclosed and described in U.S. Pat. No. 5,011,912, incorporated herein by reference. For example, fusion peptides or proteins expressing multiple antigenic domains may be separated from cells which do not express the same by using anti-Tag₁ or anti-Tag₂ antibodies which are coupled to beads by means of a hydrazide linkage. Such methods are generally described with respect to the use of the FLAG® peptide in Brizzard et al., BioTechniques, 16: 730 (1994), hereby incorporated herein by reference. By way of example, to accomplish separation using this affinity separation technique, a sample containing the fusion peptide or protein of the present invention is mixed with beads which are coupled to an anti-Tag₁ antibody. Fusion peptides or proteins expressing Tag₁ will bind to the antibodies coupled to the beads, while peptides or proteins not expressing Tag₁ will not. The proteins or peptides bound to the beads can then be recovered by, for example, centrifugation. The pellet is then resuspended and subjected to a second round of immunoprecipitation wherein the suspension is mixed with beads which are coupled to an anti-Tag₂ antibody. Fusion peptides or proteins expressing Tag₂ will bind to the antibodies coupled to the beads, while peptides or proteins not expressing Tag₂ will not. The proteins or peptides bound to the beads can then be recovered by, for example, a second round of centrifugation. Alternatively, subsequent to mixing the fusion peptide or protein with beads coupled to an anti-Tag₁ antibody, but prior to centrifugation of the same, the anti-body bound fusion peptide or protein may be mixed with beads which are coupled to a an anti-Tag₂ antibody, thereby resulting in fusion peptides or proteins that are bound to both beads coupled to anti-Tag₁ antibodies and to beads coupled to anti-Tag₂ antibodies. The peptides or proteins bound to the beads may then be recovered by, for example, centrifugation.

Another well-known affinity technique is to couple a ligand, such as biotin, to antibodies having an affinity for a fusion peptide or protein of the present invention. For example, antibodies to the fusion peptides or proteins of present invention may be coupled to biotin by a hydrazide linkage. The fusion peptides or proteins of the present invention may then be separated from peptides or proteins that do not express Tag₁ or Tag₂ through the use of avidin or streptavidin attached to magnetic beads. When the sample is placed in a magnetic field only the peptides or proteins of the present invention expressing Tag₁ or Tag₂ will bind to the magnetic beads via the linkage between the anti-Tag₁ or anti-Tag₂ antibody and the bonds between, for example, the biotin and avidin. The peptides or proteins attached to the beads can be recovered and the others washed away. Of course, this process may be performed once using antibodies directed to Tag₁ or Tag₂ and then performed a second time, either before or after a wash, using antibodies directed to the Tag not utilized in the first separation. The peptides or proteins attached to the beads can be recovered and the others washed away.

Other methods of detection, identification, isolation, capture, and/or purification of tagged fusion peptides or proteins are well known in the art, as demonstrated in “Principles and Practice of Immunoassay,” Price and Newman, eds., Stochton Press (1991), Molecular Cloning, A Laboratory Manual, 3rd Edition, Sambrook et al. eds., Cold Spring Harbor Press (2001) and “Antibodies, A Laboratory Manual,” Harlow, E. and Lane, D., eds. (1988) Cold Spring Harbor Press.

Accordingly, one embodiment of the present invention is a process capturing a polypeptide, protein, or protein fragment in or from a sample, the process comprising combining an antibody specific for one or more of the antigenic domains of the polypeptide, protein, or protein fragment of the invention with the sample containing the polypeptide, protein, or protein fragment of the invention to bind the polypeptide, protein or protein fragment of the invention to the antibody. The antibody may be either mobilized or immobilized and may be labeled or unlabeled. The process may further comprise repeating the process utilizing a second antibody specific for an antigenic domain of the polypeptide, protein, or protein fragment of the invention not utilized in the first detection, identification, isolation, capture or purification process. The process may further comprise releasing the polypeptide, protein, protein fragment, or a portion thereof from either or both of the antibodies.

Another embodiment of the present invention is a process for capturing a polypeptide, protein, or protein fragment of the present invention from a sample, the process comprising combining the sample with an antibody or receptor of Tag₁ to bind the polypeptide, protein, or protein fragment to the antibody or receptor; eluting the polypeptide, protein, protein fragment, or a portion thereof, from the antibody or receptor of Tag₁ to form a first eluant containing the polypeptide, protein, protein fragment, or a portion thereof; combining the first eluant with an antibody or receptor of Tag₂ to bind the polypeptide, protein, or protein fragment to the antibody or receptor; and eluting the polypeptide, protein, protein fragment, or a portion thereof, from the antibody or receptor of Tag₂ to form a second eluant containing the polypeptide, protein, or protein fragment, or a portion thereof. The eluant may be formed by eluting the polypeptide, protein, protein fragment, or a portion thereof, from the antibody or receptor of Tag₁, Tag₂, or both Tag₁ and Tag₂ by using an excess of a polypeptide, protein, or protein fragment that competitively binds to the antibody or receptor of Tag₁, Tag₂, or both Tag₁ and Tag₂. The eluant may also be formed by cleaving the polypeptide, protein, protein fragment, or a portion thereof, from the antibody or receptor of Tag₁, Tag₂, or both Tag₁ and Tag₂. The antibody or receptor may be either separated from the solid support or material to which it is attached (thereby resulting in an eluant that contains both the peptide of interest as well as one or more of the tags and the antibody or receptor) or may remain attached to the solid support or material, the peptide instead being released from the antibody or receptor (thereby resulting in an eluant that contains only the protein of interest and one or more of the tags).

In another embodiment wherein the recombinant polypeptide, protein, or protein fragment also contains a metal ion-affinity tag, the process for capturing a polypeptide, protein, or protein fragment in or from a sample additionally comprises combining the polypeptide, protein, protein fragment, or a portion thereof with an immobilized metal ion. This may occur either before or after the polypeptide, protein, or protein fragment in or from the sample is combined with one or more antibodies. The polypeptide, protein, or protein fragment may thereafter be selectively released from immobilized metal. For example, if there is a cleavage site between the target polypeptide, protein or protein fragment and the metal ion-affinity peptide, and if the bound recombinant polypeptide, protein or protein fragment is treated with the appropriate enzyme, the target polypeptide, protein or protein fragment may be selectively released while the metal ion-affinity polypeptide fragment remains bound to the immobilized metal. For this purpose, the cleavage is preferably an enzymatically cleavable linker peptide having the ability to undergo site-specific proteolysis. Suitable cleaving enzymes in accordance with this invention are activated factor X (factor Xa), DPP I, DPP II, DPP IV, carboxylpeptidase A, collagen, enterokinase, human renin, thrombin, trypsin, subtilisn and V5.

It is to be appreciated by one skilled in the art that some polypeptide or protein molecules will possess the desired enzymatic or biological activity with the metal chelate peptide still attached either at the C-terminal end or at the N-terminal end or both. In those cases the purification of the chimeric protein will be accomplished without subjecting the protein to site-specific proteolysis. In such an instance, the polypeptide or protein molecule may be released from the immobilized metal by competition with another free or mobile metal ion that either competitively binds the immobilized metal ions of the solid support or competitively binds the metal affinity-ion tag of the recombinant polypeptide, protein, or protein fragment. Alternatively,

The present invention may be used to purify any prokaryotic or eukaryotic protein that can be expressed as the product of recombinant DNA technology in a transformed host cell. These recombinant protein products include hormones, receptors, enzymes, storage proteins, blood proteins, mutant proteins produced by protein engineering techniques, or synthetic proteins. The purification process of the present invention can be used batch-wise or in continuously run columns.

The present invention may also be used to capture, and subsequently isolate, purify, and identify, an unknown peptide protein using a recombinant polypeptide, protein, or protein fragment of the invention, wherein a peptide sequence (bait peptide or protein) contained in the recombinant polypeptide, protein, or protein fragment of the invention binds to the unknown protein forming a protein complex between the bait peptide or protein and the unknown peptide or protein. The protein complex may then be captured, and subsequently detected, identified, isolated, or purified, based upon binding of the antigenic domains contained in the bait peptide or protein to one or more antibodies or receptors having affinity for an antigenic domain of the bait peptide or protein.

Having described the invention in detail, it will be apparent and appreciated by one of skill in the art that modifications and variations are possible without departing from the scope of the invention defined in the appended claims.

### EXAMPLES

The following non-limiting examples are provided to further illustrate and clarify the present invention. It should be appreciated by those of skill in the art that the techniques disclosed in the examples that follow, represent approaches the inventors have found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit, scope and claims of the invention.

### Example 1

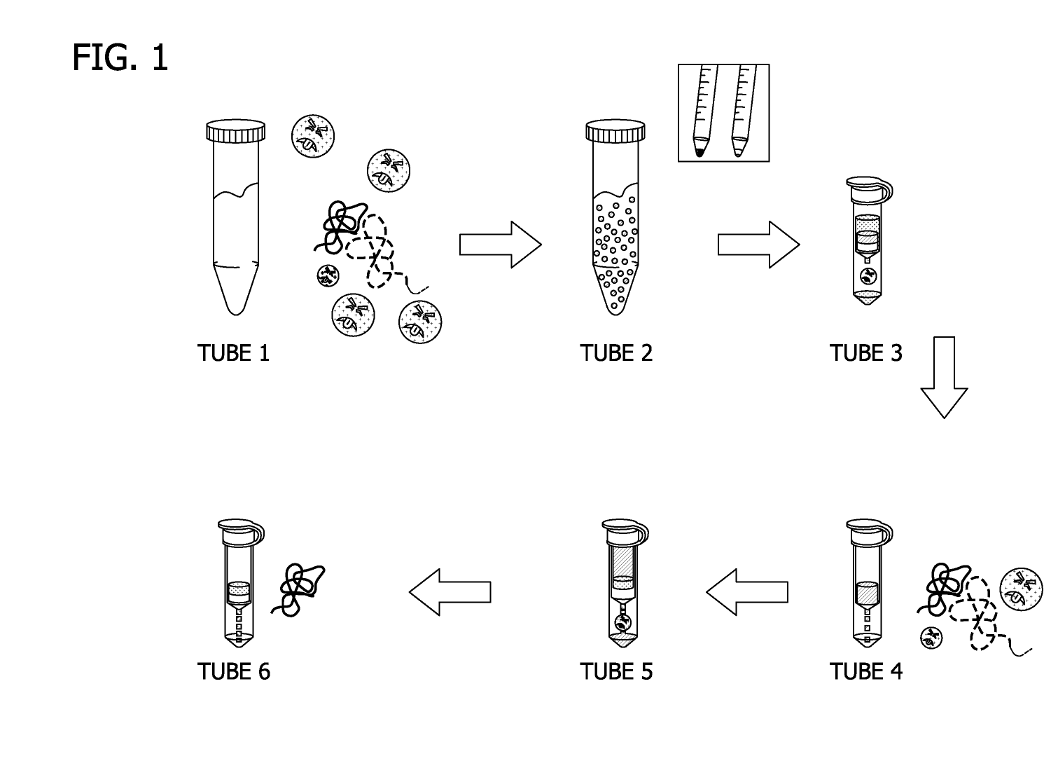

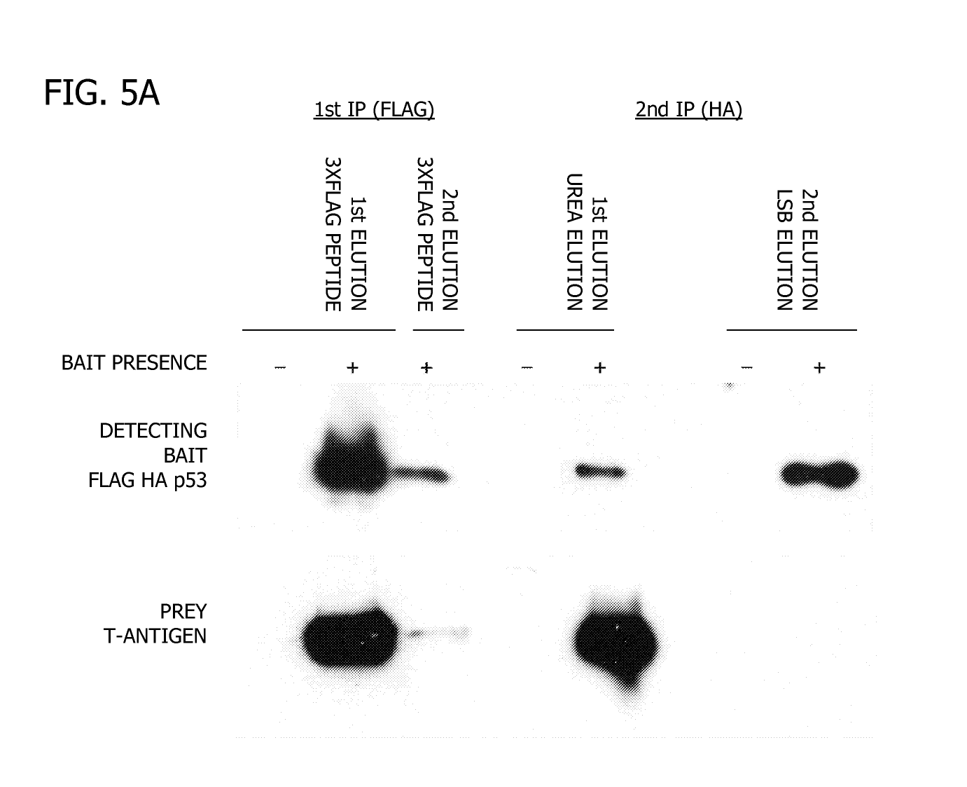

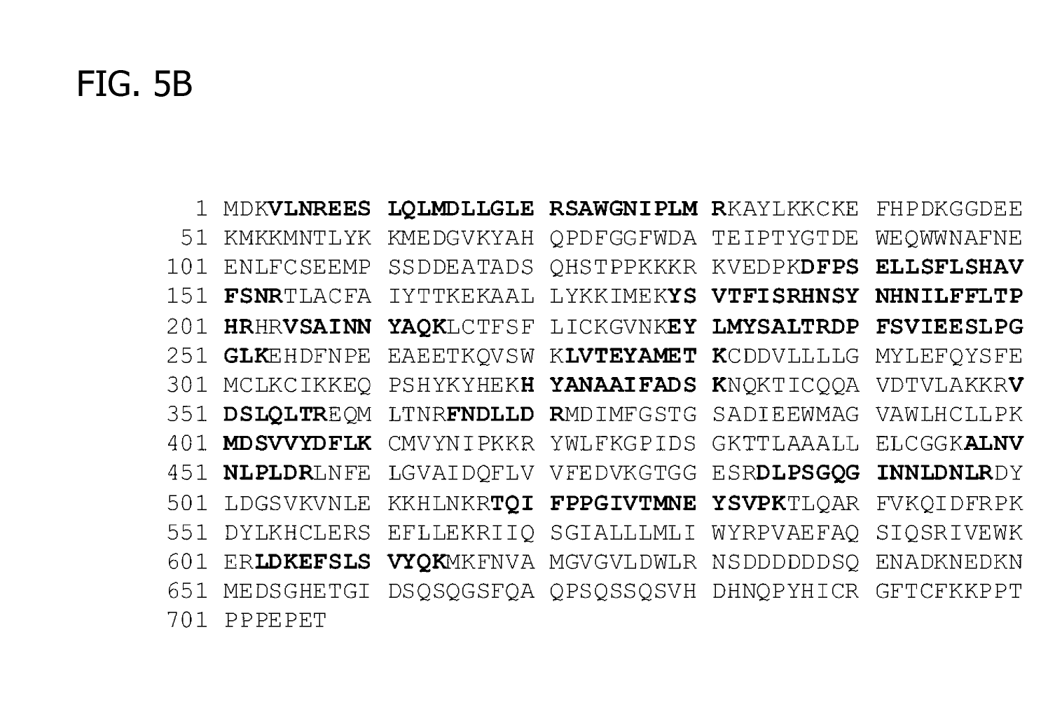

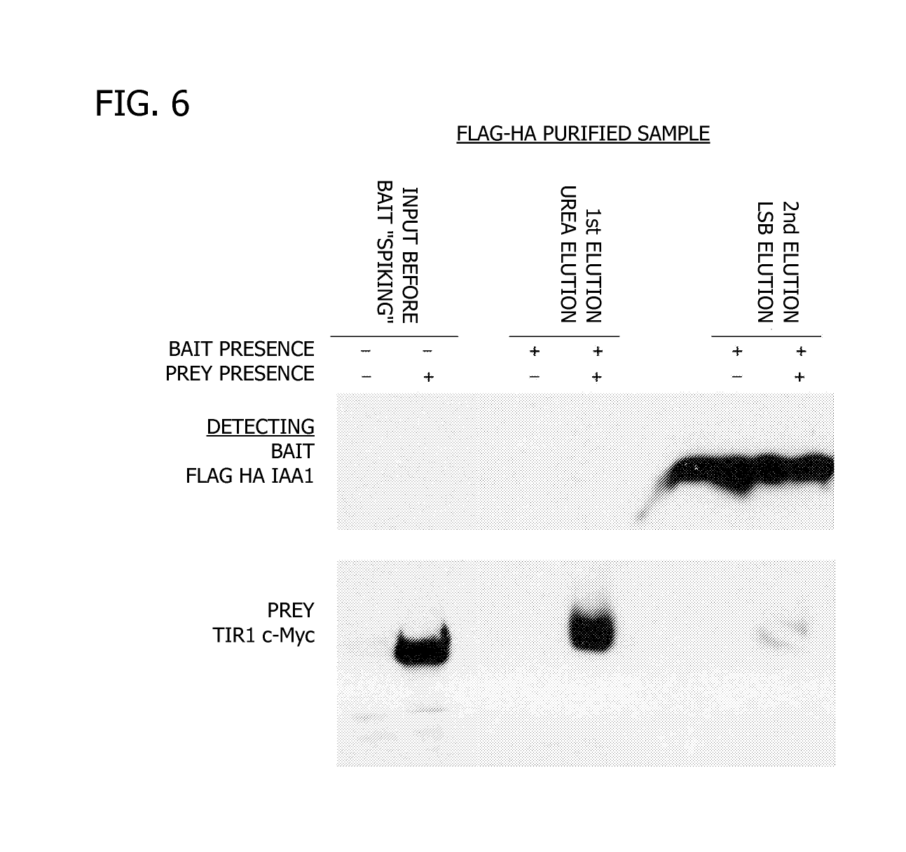

A schematic representation of the FLAG® HA Tandem Affinity Purification system is illustrated in FIG. 1. The protein of interest is tandem tagged with FLAG® HA epitopes and referred to as the bait protein. Endogenous protein complexes (referred to as prey complex) interact with the tandem tagged bait protein and will be sequentially co-purified. The whole procedure involves six simple steps: 1) Prepare the starting material that contains FLAG® HA tagged bait protein; this is normally conducted in 15 ml conical tubes. Extracts should be prepared in an immunoprecipitation-compatible buffer, such as RIPA (150 mM NaCl, 1.0% Igepal CA-630, 0.5% sodium deoxycholate, 0.1% SDS, 50 mM Tris, pH 8.0) and with proteinase inhibitors. The bait protein can be expressed in vivo (see FIG. 5) or expressed in bacteria, purified, and added exogenously to extract (see FIG. 6); 2) Add EZview ANTI-FLAG® resin directly to the lysate and incubate at least for 2 hours at 4° C. The red color of the resin provides better visibility during incubation and for transfer (see sub-panel); 3) transfer the resin to a spin column (˜700 ul) and wash with RIPA by agitating to resuspend the resin; 4) conduct 1^{st} elution with 150 ng/μl 3×FLAG® peptide in TBS (Tris buffered saline) for 10 minutes. This elution strategy is very efficient and mild and would not interrupt protein complex, multiple 3×FLAG® peptide elutions increases yield but the returns are dramatically reduced after 3 times; 5) Transfer the 3×FLAG® eluate (pool) directly to a spin column containing anti-HA affinity resin, bind and wash with RIPA; and 6) Conduct the 2^{nd} elution with one of the three different elution buffers (8Murea, 1 μg/μl of HA peptide, or 2× Laemmli Sample Buffer (LSB)) depending on downstream manipulations. Elute for 10 minutes at room temperature. The advantages of the urea elution buffer are its compatibility with MS analysis (data not shown) and that it preferentially elutes the prey proteins so that contamination from the bait protein is minimized (see FIGS. 5 & 6). The HA peptide elution strategy is the mildest elution, suitable for recovery of single dual-tagged proteins or protein complexes. The LSB elution strategy is the harshest. Protein from the resin will be eluted too. It is useful for analysis of tagged proteins and protein complexes by PAGE gel and Western blotting.

### Example 2

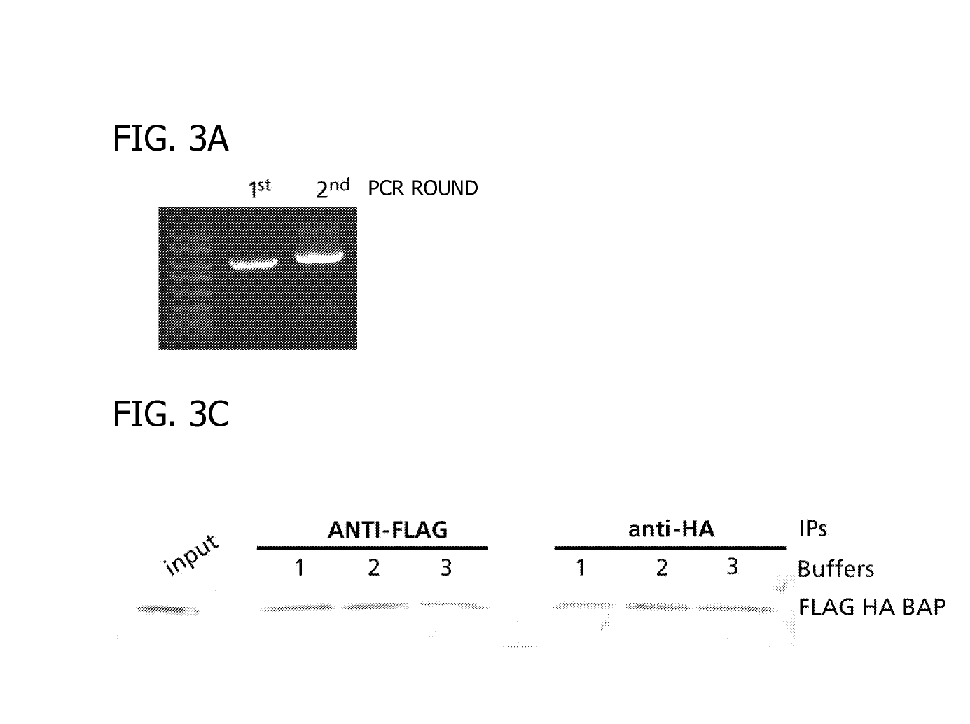

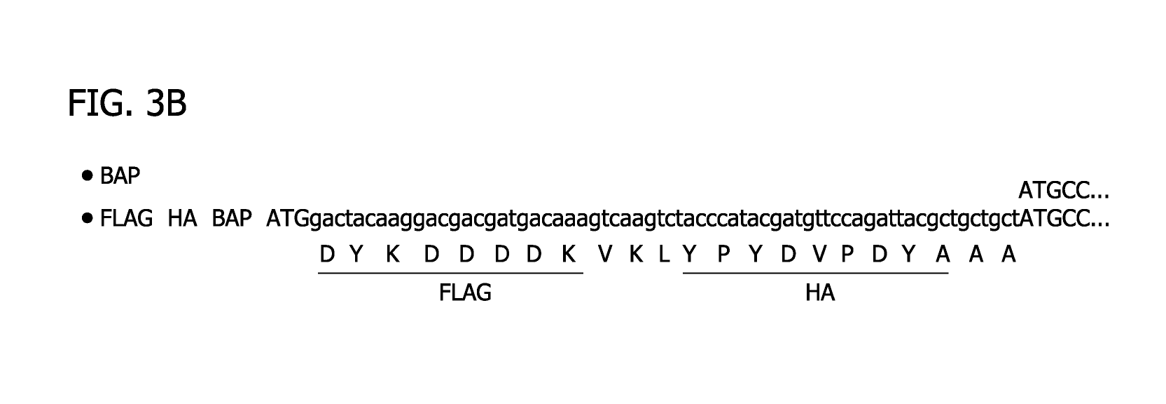

The TAP Tag Generation System allows for the rapid generation of ligation-ready DNA inserts that can be used with any expression vector to produce N-terminal FLAG®-HA fusion proteins. The whole process of generating DNA inserts is completed in four simple steps. The first step is using gene-specific primers to amplify the gene of interest. Two unique additions are required for these gene-specific primers, one is to add a 20 base pair sequence corresponding to the HA tag to the gene-specific forward primer (GSP-F). The other addition is a five base pair sequence corresponding to restriction site of choice to the gene-specific reverse primer (GSP-R). The restriction site is required for cloning of DNA inserts into expression vectors. The second step is to conduct PCR using the FLAG®-HA anchor, an anchor primer, and the GSP-R. The FLAG®-HA anchor is a double-stranded oligonucleotide containing FLAG® and HA sequences and an overhang that is complimentary to the HA overhang in the GSP-F. The anchor primer contains complementary sequence to the FLAG®-HA anchor and a restriction enzyme overhang. The choice of three overhangs, BamH I, EcoR I, and Xba I, are included in the kit to provide flexibility in choosing expression vectors. The third step is to digest PCR product with Exonuclease III to create cohesive ends for cloning. Using Exonuclease III digestion instead of conventional restriction enzyme digestion alleviates the concern about internal restriction sites in DNA inserts. To prevent over-digestion of Exonuclease III, a proprietary dNTP mix that contains dATPαS and dGTPαS is provided in the kit for PCR. This specially formulated dNTP mix has been optimized so that dATPαS and dGTPαS can be randomly incorporated into PCR products in a ratio that provides protection from Exonuclease III digestion and ensures consistency in creation of cohesive ends. The final step is to ligate dual-tagged DNA inserts to a double-digested expression vector of choice, which contains overhangs complementary to those in the DNA inserts. FIG. 3 shows the creation of and immunoprecipitations with FLAG® HA protein-of-interest.

### Example 3

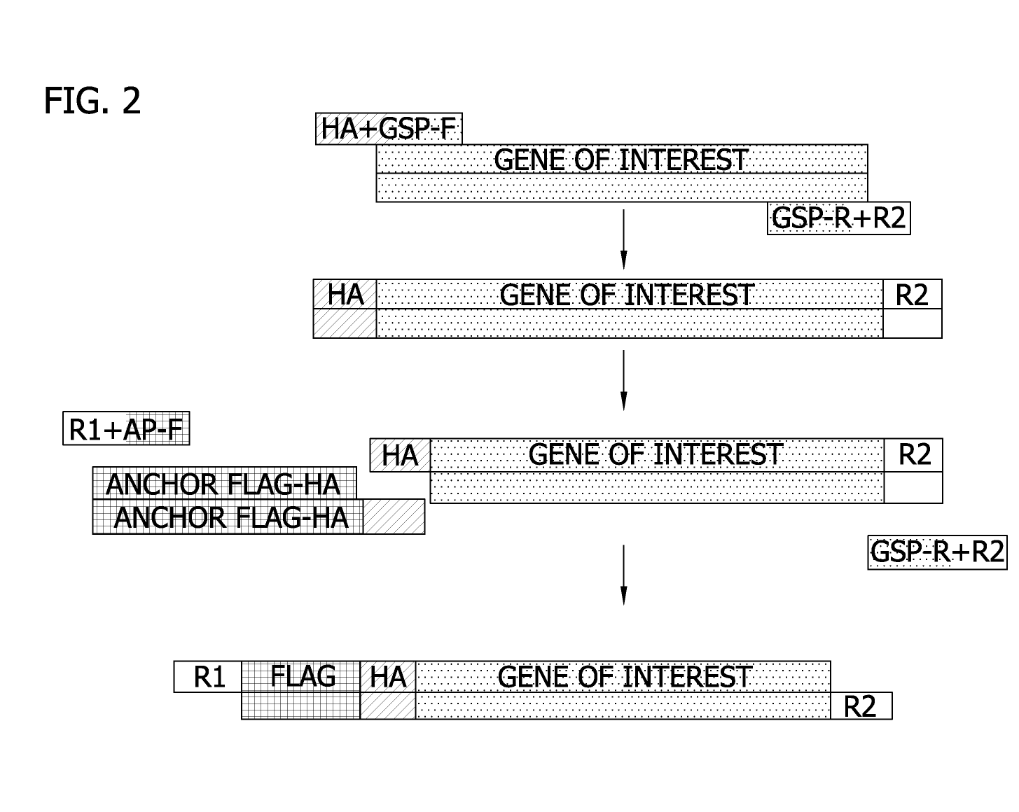

Both ANTI-FLAG® and anti-HA resins reproducibly capture a high percentage of tandem tagged protein with different buffer systems. FIG. 3A demonstrates that the FLAG® HA tandem tag was incorporated into bacterial alkaline phosphatase (BAP) using the FLAG® HA TAP Tag Generation Kit (see FIG. 2) and the amplicon ligated into a bacterial expression vector (MAC, RDCLIG1-1KT). FIG. 3B discloses the nucleic acid and peptide sequence of the FLAG® HA BAP fusion protein. The FLAG® and HA epitopes are underlined. FIG. 3C demonstrates that the dual tagged FLAG®-HA BAP protein was expressed in and purified from the bacteria host, BL21. Purified FLAG® HA BAP was incubated in 3 different extraction buffers (1-3) at 4° C. for at least 30 minutes and immunoprecipitated using ANTI-FLAG® and anti-HA affinity resins, separately. Protein recovery from both ANTI-FLAG® and anti-HA affinity resins is greater or equal to 70% of input protein. 150 ng/113×FLAG® peptide does not interfere with anti-HA IP (data not shown). Detection was shown with Anti-HA-Peroxidase conjugated antibody.

### Example 4

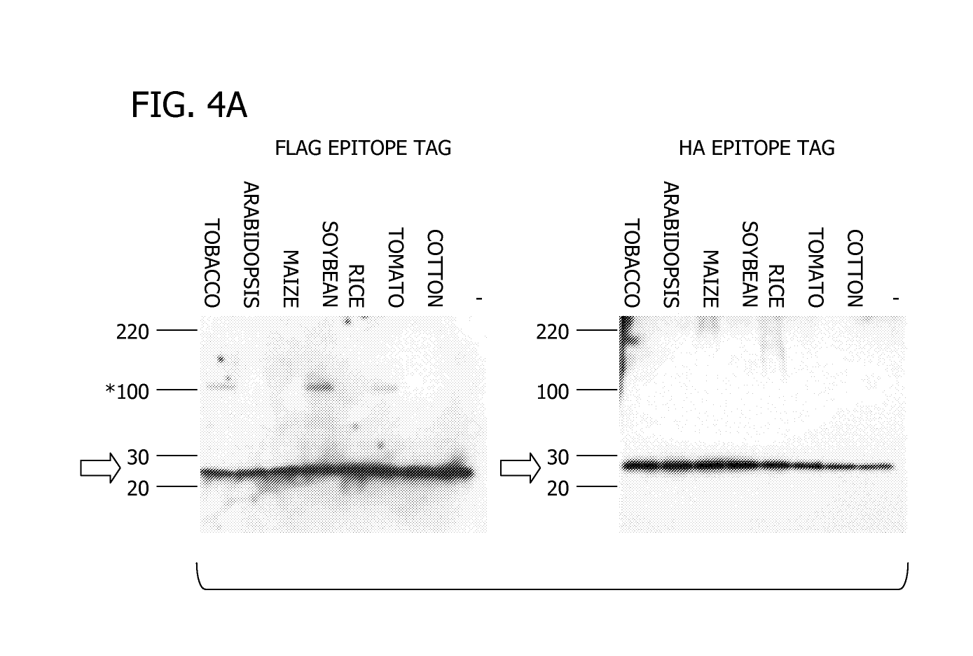

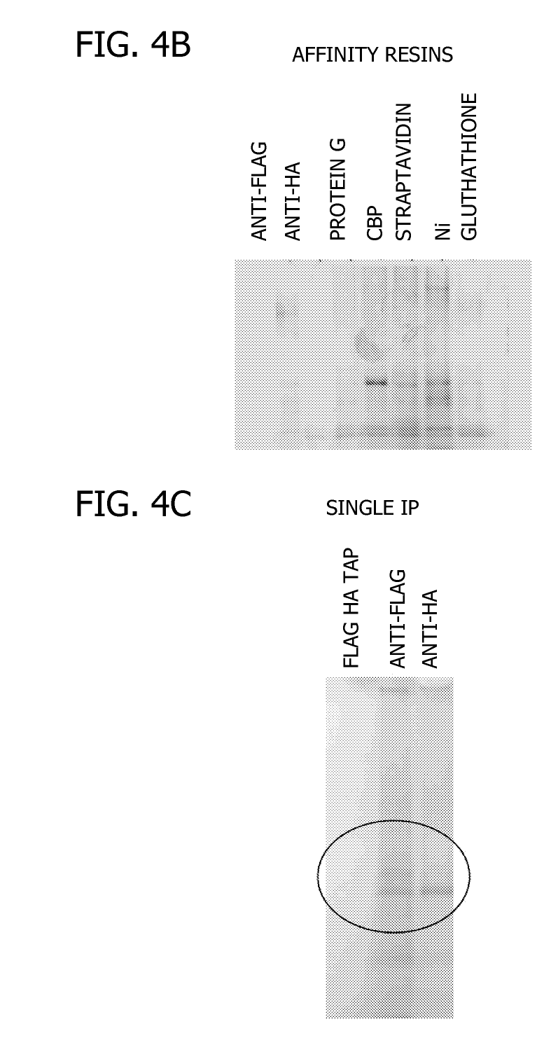

Specificity analysis of FLAG® and HA tags and affinity resins using plant materials. FIG. 4A is a gel showing the high specificity of the ANTI-FLAG® and anti-HA antibodies to FLAG®- and HA-tagged fusion proteins, respectively, in 7 different plant extracts. 0.1 μg of epitope-tagged proteins (FLAG® GST or GST HA) were spiked into 20 μg of total leaf proteins extracted from tobacco, Arabidopsis, maize, soybean, rice, tomato, and cotton. Chemilumenscent blot detection was with ANTI-FLAG® M2-AP and anti-HA-HRP antibody conjugates and their respective substrates. FIG. 4B is a gel showing the specificity of ANTI-FLAG® and anti-HA affinity resins in comparison with existing resins. 40 μl of resin slurries (ANTI-FLAG®, anti-HA, Protein G, CBP, Streptavidin, Nickel, and Glutathione) were incubated with 6 mg total leaf protein from light grown Arabidopsis 4° C. for 2 hours. After washing with RIPA twice followed by a TBS wash, proteins that were non-specifically bound to the resins were eluted, resolved on PAGE gel, and detected by silver stain. Both ANTI-FLAG® and anti-HA resin showed minimal cross-reactivity compared to other resins. FIG. 4C is a gel illustrating that consecutive immunoprecipitations provide a higher purity of sample compared to a single immunoprecipitation. 8 mg of Arabidopsis leaf protein was immunoprecipitated with FLAG® then HA (FLAG® HA TAP) or FLAG® and HA alone. After incubation, resins were washed 3× with RIPA, then a TBS wash, eluted with FLAG® or HA peptide and silver stained on a PAGE to see contaminants.

### Example 5

A well-known protein-protein interaction system in mammalian cells, p53 and T-antigen, was used to characterize the FLAG® HA TAP system. FLAG HA tandem tag was incorporated into p53 (see FLAG® HA Tandem TAP Tag Generation Kit TP 0020) and transiently expressed in COS-7 cells (a mammalian cell line). The tagged p53, used as the bait protein, interacts with the endogenous T-antigen to form a complex. The protein complex was isolated using the TAP procedure. FIG. 5A illustrates two major features: one is that FLAG® HA tandem tag does not interfere with the interaction between the bait and the prey, and the other is that the urea solution preferentially elutes the prey protein (T-antigen) and leaves the bait protein (p53) on the affinity resin. The first immunoprecipitation (IP) was conducted using ANTI-FLAG® resin and the protein complex was eluted with 3×FLAG® peptide (once or twice). Both the bait and the prey were eluted off the resin equally. The second IP was conducted using anti-HA resin and eluted by urea solution followed by LSB. Almost all prey protein was eluted by urea, whereas the bait protein remains on the resin and only elutes with the addition of Laemmli Sample Buffer. Sample volumes loaded on each gel were normalized. Blots were probed with ANTI-FLAG® M2-Peroxidase or anti-T-antigen biotin (BD Biosciences)/streptavidin peroxidase. The TAP procedure was performed on COS-7 cells expressing FLAG® HA p53 and the final elution was with urea. The elution was run on a PAGE gel and silver stained. The protein band cut out was identified with MS/MS as T-antigen, the targeted “prey”, the sequence of which is disclosed in FIG. 5B.

### Example 6

Further characterization of the FLAG® HA TAP system was performed using a protein-protein interaction model in plants, the Aux/IAA-TIR1 interaction (Gray, W. M. et al., Nature 414: 271-276 (2001) and Dharmasiri, N. et al., Current Biology 13: 1418-1422 (2003)). TIR1 is an auxin receptor (Dharmasiri, N. et al., Nature 435: 441-445 (2005)). Auxin increases the affinity TIR1 has for Aux/IAAs (IAA1 is a family member) (Gray, W. M. et al., Nature 414: 271-276 (2001)). FLAG® HA IAA1 (bait protein) was expressed in bacteria and purified using ANTI-FLAG® resin and 3×FLAG® peptide (data not shown). The purified FLAG® HA IAA1 protein was incubated with protein extracts from wild type Arabidopsis or from transgenic Arabidopsis plant expressing TIR1 c-Myc in the presence of auxin. Samples were then purified using ANTI-FLAG® followed by anti-HA resin. Anti-HA resin was eluted with urea (to elute the prey protein) then LSB (to elute the bait protein). Blots were probed with ANTI-FLAG® antibody to detect FLAG®-HA tagged IAA1 (the bait) or with anti-c-myc antibody to detect c-myc-tagged TIR1 (the prey). Similar to p53/T-antigen model, the prey protein is predominantly eluted with the urea elution, while the bait protein is not.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained. As various changes could be made in the above polypeptides, proteins, or protein fragments or the methods of making or using the same without departing from the scope of the invention, it is intended that all matter contained in the above description and shown in the accompanying drawing[s] shall be interpreted as illustrative and not in a limiting sense.

What is claimed is:

1. A nucleic acid sequence encoding for a polypeptide, a protein, or a protein fragment represented by a formula comprising:
R₁-Sp₁-Tag₁-Sp₂-R₂-Sp₃-Tag₂-Sp₄-R₃
wherein,
(a) R₁ is hydrogen, a polypeptide, a protein, or a protein fragment;
(b) Sp₁ is a bond or a spacer comprising at least one amino acid residue;
(c) Tag₁ is an antigenic domain, said antigenic domain comprising at least one antigenic determinant selected from the group consisting of (i) a peptide sequence having specificity for an antibody to the peptide sequence DYKDDDDK (SEQ ID NO: 1), (ii) a peptide sequence having specificity for an antibody to the peptide sequence DLYDDDDK (SEQ ID NO: 2), (iii) the HA epitope (SEQ ID NO: 3), (iv) c-myc (SEQ ID NO: 5), (v) AcV5 (SEQ ID NO: 4); and (vi) a peptide sequence having specificity for streptavidin or a streptavidin derivative;
(d) Sp₂ is a bond or a spacer comprising at least one amino acid residue;
(e) R₂ is a bond, a polypeptide, a protein, or a protein fragment;
(f) Sp₃ is a bond or a spacer comprising at least one amino acid;
(g) Tag₂ is an antigenic domain, said antigenic domain comprising at least one antigenic determinant selected from the group consisting of (i) a peptide sequence having specificity for an antibody to the peptide sequence DYKDDDDK (SEQ ID NO: 1), (ii) a peptide sequence having specificity for an antibody to the peptide sequence DLYDDDDK (SEQ ID NO: 2), (iii) the HA epitope (SEQ ID NO: 3), (iv) c-myc (SEQ ID NO: 5), (v) AcV5 (SEQ ID NO: 4); and (vi) a peptide sequence having specificity for streptavidin or a streptavidin derivative;
(h) Sp₄ is a bond or a spacer comprising at least one amino acid; and
(i) R₃ is hydrogen, a polypeptide, a protein, or a protein fragment; wherein, Tag₁ and Tag₂ are independently different antigenic domains.

2. A vector comprising the nucleic acid sequence of claim 1.

3. A host cell comprising the nucleic acid sequence of claim 1.

4. A host cell comprising the vector of claim 2.

5. The nucleic acid of claim 1, wherein the peptide sequence having specificity for an antibody to the peptide sequence DYKDDDDK (SEQ ID NO: 1) is the M1 antibody, the M2 antibody, or the M5 antibody.

6. The nucleic acid of claim 1, wherein the antigenic domain of Tag₁ or Tag₂ is a peptide sequence selected from the group consisting of:

| | | |
|---|---|---|
| (a) | DYKDDDDK; | (SEQ ID NO: 1) |
| (b) | DYKDHDGDYKDHDIDYKDDDDK; | (SEQ ID NO: 6) |
| (c) | MDYKDHDGDYKDHDIDYKDDDDK; | (SEQ ID NO: 13) |
| (d) | DLYDDDDK; | (SEQ ID NO: 2) |
| and | | |
| (e) | DLYDHDGDLYDHDIDLYDDDDK. | (SEQ ID NO: 7) |

7. The nucleic acid of claim 1, wherein the peptide sequence having specificity for streptavidin or a streptavidin derivative is selected from the group consisting of the SBP-tag, the S1 Aptamer, the C-Terminal streptavidin binding tag, the Nano-tag, strep tag I, and strep tag II.

8. The nucleic acid of claim 1, wherein Tag₁ and Tag₂ comprise at least one copy of an antigenic domain selected from the group consisting of DYKDDDDK (SEQ ID NO: 1) or the HA epitope.

9. The nucleic acid of claim 12, wherein Tag₁ and Tag₂ comprise at least one copy of an antigenic domain selected from the group consisting of DYKDDDDK (SEQ ID NO: 1) or c-myc.

10. The nucleic acid of claim 1, wherein Tag₁ and Tag₂ comprise at least one copy of an antigenic domain selected from the group consisting of DYKDDDDK (SEQ ID NO: 1) or AcV5.

11. The nucleic acid of claim 1, wherein Tag₁ and Tag₂ are selected from the group consisting of DYKDHDGDYKDHDIDYKDDDDK (SEQ ID NO: 6) or DLYDHDGDLYDHDIDLYDDDDK (SEQ ID NO: 7).

12. The nucleic acid of claim 1, wherein Tag₁ and Tag₂ are selected from the group consisting of DYKDHDGDYKDHDIDYKDDDDK (SEQ ID NO: 6) or the HA epitope.

13. The nucleic acid of claim 1, wherein Tag₁ and Tag₂ are selected from the group consisting of DYKDHDGDYKDHDIDYKDDDDK (SEQ ID NO: 6) or c-myc.

14. The nucleic acid of claim 1, wherein Tag₁ and Tag₂ are selected from the group consisting of DYKDHDGDYKDHDIDYKDDDDK (SEQ ID NO: 6) or AcV5.

15. The nucleic acid of claim 1, wherein Tag₁ and Tag₂ are selected from the group consisting of DYKDHDGDYKDHDIDYKDDDDK (SEQ ID NO: 6) or YPYDVPDYAYPYDVPDYA (SEQ ID NO: 8).

16. The nucleic acid of claim 1, wherein Tag₁ and Tag₂ are selected from the group consisting of DYKDHDGDYKDHDIDYKDDDDK (SEQ ID NO: 6) or YPYDVPDYAYPYDVPDYAYPYDVPDYA (SEQ ID NO: 9).

17. The nucleic acid of claim 1, wherein when Tag₁ is a peptide sequence having specificity for an antibody to the peptide sequence DYKDDDDK (SEQ ID NO: 1), and Tag₂ is selected from a group consisting of (i) a peptide sequence having specificity for an antibody to the peptide sequence DLYDDDDK (SEQ ID NO: 2), (ii) the HA epitope, (iii) c-myc, (iv) AcV5, and (v) a peptide sequence having specificity for streptavidin or a streptavidin derivative.

18. The nucleic acid of claim 1, wherein when Tag₁ is a peptide sequence having specificity for an antibody to the peptide sequence DLYDDDDK (SEQ ID NO: 2), and Tag₂ is selected from a group consisting of (i) a peptide sequence having specificity for an antibody to the peptide sequence DYKDDDDK (SEQ ID NO: 1), (ii) the HA epitope, (iii) c-myc, (iv) AcV5, and (v) a peptide sequence having specificity for streptavidin or a streptavidin derivative.

19. The nucleic acid of claim 1, wherein when Tag₁ is the HA epitope, Tag₂ is selected from a group consisting of (i) a peptide sequence having specificity for an antibody to the peptide sequence DLYDDDDK (SEQ ID NO: 2), (ii) a peptide sequence having specificity for an antibody to the peptide sequence DYKDDDDK (SEQ ID NO: 1), (iii) c-myc, (iv) AcV5, and (v) a peptide sequence having specificity for streptavidin or a streptavidin derivative.

20. The nucleic acid of claim 1, wherein when Tag₁ is c-myc, and Tag₂ is selected from a group consisting of (i) a peptide sequence having specificity for an antibody to the peptide sequence DLYDDDDK (SEQ ID NO: 2), (ii) a peptide sequence having specificity for an antibody to the peptide sequence DYKDDDDK (SEQ ID NO: 1), (iii) the HA epitope, (iv) AcV5, and (v) a peptide sequence having specificity for streptavidin or a streptavidin derivative.

21. The nucleic acid of claim 1, wherein when Tag₁ is AcV5, and Tag₂ is selected from a group consisting of (i) a peptide sequence having specificity for an antibody to the peptide sequence DLYDDDDK (SEQ ID NO: 2), (ii) a peptide sequence having specificity for an antibody to the peptide sequence DYKDDDDK (SEQ ID NO: 1), (iii) the HA epitope, (iv) c-myc, and (v) a peptide sequence having specificity for streptavidin or a streptavidin derivative.

22. The nucleic acid of claim 1, wherein when Tag₁ is a peptide sequence having specificity for streptavidin or a streptavidin derivative, and Tag₂ is selected from a group consisting of (i) a peptide sequence having specificity for an antibody to the peptide sequence DLYDDDDK (SEQ ID NO: 2), (ii) a peptide sequence having specificity for an antibody to the peptide sequence DYKDDDDK (SEQ ID NO: 1), (iii) the HA epitope, (iv) c-myc, and (v) AcV5.

23. The nucleic acid of claim 1, wherein Tag₁ and Tag₂ are, in combination, selected from combinations 1-190 of the following table:

| | | |
|---|---|---|
| Combo # | Tag₁ | Tag₂ |
| | | |
|---|---|---|
| 1 | DYKDDDDK | DLYDDDDK |
| | (SEQ ID NO: 1) | (SEQ ID NO: 2) |
| 2 | DYKDDDDK | YPYDVPDYA |
| | (SEQ ID NO: 1) | (SEQ ID NO: 3) |
| 3 | DYKDDDDK | SWKDASGWS |
| | (SEQ ID NO: 1) | (SEQ ID NO: 4) |
| 4 | DYKDDDDK | EQKLISEEDL |
| | (SEQ ID NO: 1) | (SEQ ID NO: 5) |
| 5 | DYKDDDDK | AWRHPQFGG |
| | (SEQ ID NO: 1) | (SEQ ID NO: 19) |
| 6 | DYKDDDDK | WSGPQFEK |
| | (SEQ ID NO: 1) | (SEQ ID NO: 20) |
| 7 | DYKDDDDK | DLYDDDDKDLYDDDDK |
| | (SEQ ID NO: 1) | (SEQ ID NO: 30) |
| 8 | DYKDDDDK | YPYDVPDYAYPYDVPDYA |
| | (SEQ ID NO: 1) | (SEQ ID NO: 8) |
| 9 | DYKDDDDK | SWKDASGWSSWKDASGWS |
| | (SEQ ID NO: 1) | SEQ ID NO: 10) |
| 10 | DYKDDDDK | EQKLISEEDLEQKLISEEDL |
| | (SEQ ID NO: 1) | (SEQ ID NO: 36) |
| 11 | DYKDDDDK | AWRHPQFGGAWRHPQFGG |
| | (SEQ ID NO: 1) | (SEQ ID NO: 21) |
| 12 | DYKDDDDK | WSGPQFEKWSGPQFEK |
| | (SEQ ID NO: 1) | (SEQ ID NO: 23) |
| 13 | DYKDDDDK | DLYDDDDKDLYDDDDKDLYDDDDK |
| | (SEQ ID NO: 1) | (SEQ ID NO: 31) |
| 14 | DYKDDDDK | YPYDVPDYAYPYDVPDYAYPYDVPDYA |
| | (SEQ ID NO: 1) | (SEQ ID NO: 9) |
| 15 | DYKDDDDK | SWKDASGWSSWKDASGWSSWKDASGWS |
| | (SEQ ID NO: 1) | (SEQ ID NO: 11) |
| 16 | DYKDDDDK | EQKLISEEDLEQKLISEEDLEQKLISEEDL |
| | (SEQ ID NO: 1) | (SEQ ID NO: 37) |
| 17 | DYKDDDDK | AWRHPQFGGAWRHPQFGGAWRHPQFGG |
| | (SEQ ID NO: 1) | (SEQ ID NO: 22) |
| 18 | DYKDDDDK | WSGPQFEKWSGPQFEKWSGPQFEK |
| | (SEQ ID NO: 1) | (SEQ ID NO: 24) |
| 19 | DYKDDDDK | DLYDHDGDLYDHDIDLYDDDDK |
| | (SEQ ID NO: 1) | (SEQ ID NO: 7) |
| 20 | DYKDDDDKDYKDDDDK | DLYDDDDK |
| | (SEQ ID NO: 25) | (SEQ ID NO: 2) |
| 21 | DYKDDDDKDYKDDDDK | YPYDVPDYA |
| | (SEQ ID NO: 25) | (SEQ ID NO: 3) |
| 22 | DYKDDDDKDYKDDDDK | SWKDASGWS |
| | (SEQ ID NO: 25) | (SEQ ID NO: 4) |
| 23 | DYKDDDDKDYKDDDDK | EQKLISEEDL |
| | (SEQ ID NO: 25) | (SEQ ID NO: 5) |
| 24 | DYKDDDDKDYKDDDDK | AWRHPQFGG |
| | (SEQ ID NO: 25) | (SEQ ID NO: 19) |
| 25 | DYKDDDDKDYKDDDDK | WSGPQFEK |
| | (SEQ ID NO: 25) | (SEQ ID NO: 20) |
| 26 | DYKDDDDKDYKDDDDK | DLYDDDDKDLYDDDDK |
| | (SEQ ID NO: 25) | (SEQ ID NO: 30) |
| 27 | DYKDDDDKDYKDDDDK | YPYDVPDYAYPYDVPDYA |
| | (SEQ ID NO: 25) | (SEQ ID NO: 8) |
| 28 | DYKDDDDKDYKDDDDK | SWKDASGWSSWKDASGWS |
| | (SEQ ID NO: 25) | (SEQ ID NO: 10) |
| 29 | DYKDDDDKDYKDDDDK | EQKLISEEDLEQKLISEEDL |
| | (SEQ ID NO: 25) | (SEQ ID NO: 36) |
| 30 | DYKDDDDKDYKDDDDK | AWRHPQFGGAWRHPQFGG |
| | (SEQ ID NO: 25) | (SEQ ID NO: 21) |
| 31 | DYKDDDDKDYKDDDDK | WSGPQFEKWSGPQFEK |
| | (SEQ ID NO: 25) | (SEQ ID NO: 23) |
| 32 | DYKDDDDKDYKDDDDK | DLYDDDDKDLYDDDDKDLYDDDDK |
| | (SEQ ID NO: 25) | (SEQ ID NO: 31) |
| 33 | DYKDDDDKDYKDDDDK | YPYDVPDYAYPYDVPDYAYPYDVPDYA |
| | (SEQ ID NO: 25) | (SEQ ID NO: 9) |
| 34 | DYKDDDDKDYKDDDDK | SWKDASGWSSWKDASGWSSWKDASGWS |
| | (SEQ ID NO: 25) | (SEQ ID NO: 11) |
| 35 | DYKDDDDKDYKDDDDK | EQKLISEEDLEQKLISEEDLEQKLISEEDL |
| | (SEQ ID NO: 25) | (SEQ ID NO: 37) |
| 36 | DYKDDDDKDYKDDDDK | AWRHPQFGGAWRHPQFGGAWRHPQFGG |
| | (SEQ ID NO: 25) | (SEQ ID NO: 22) |
| 37 | DYKDDDDKDYKDDDDK | WSGPQFEKWSGPQFEKWSGPQFEK |
| | (SEQ ID NO: 25) | (SEQ ID NO: 24) |
| 38 | DYKDDDDKDYKDDDDK | DLYDHDGDLYDHDIDLYDDDDK |
| | (SEQ ID NO: 25) | (SEQ ID NO: 7) |
| 39 | DYKDDDDKDYKDDDDKDYKDDDDK | DLYDDDDK |
| | (SEQ ID NO: 26) | (SEQ ID NO: 2) |
| 40 | DYKDDDDKDYKDDDDKDYKDDDDK | YPYDVPDYA |
| | (SEQ ID NO: 26) | (SEQ ID NO: 3) |
| 41 | DYKDDDDKDYKDDDDKDYKDDDDK | SWKDASGWS |
| | (SEQ ID NO: 26) | (SEQ ID NO: 4) |
| 42 | DYKDDDDKDYKDDDDKDYKDDDDK | EQKLISEEDL |
| | (SEQ ID NO: 26) | )SEQ ID NO: 5) |
| 43 | DYKDDDDKDYKDDDDKDYKDDDDK | AWRHPQFGG |
| | (SEQ ID NO: 26) | (SEQ ID NO: 19) |
| 44 | DYKDDDDKDYKDDDDKDYKDDDDK | WSGPQFEK |
| | (SEQ ID NO: 26) | (SEQ ID NO: 20) |
| 45 | DYKDDDDKDYKDDDDKDYKDDDDK | DLYDDDDKDLYDDDDK |
| | (SEQ ID NO: 26) | (SEQ ID NO: 30) |
| 46 | DYKDDDDKDYKDDDDKDYKDDDDK | YPYDVPDYAYPYDVPDYA |
| | (SEQ ID NO: 26) | (SEQ ID NO: 8) |
| 47 | DYKDDDDKDYKDDDDKDYKDDDDK | SWKDASGWSSWKDASGWS |
| | (SEQ ID NO: 26) | (SEQ ID NO: 10) |
| 48 | DYKDDDDKDYKDDDDKDYKDDDDK | EQKLISEEDLEQKLISEEDL |
| | (SEQ ID NO: 26) | (SEQ ID NO: 36) |
| 49 | DYKDDDDKDYKDDDDKDYKDDDDK | AWRHPQFGGAWRHPQFGG |
| | (SEQ ID NO: 26) | (SEQ ID NO: 21) |
| 50 | DYKDDDDKDYKDDDDKDYKDDDDK | WSGPQFEKWSGPQFEK |
| | (SEQ ID NO: 26) | (SEQ ID NO: 23) |
| 51 | DYKDDDDKDYKDDDDKDYKDDDDK | DLYDDDDKDLYDDDDKDLYDDDDK |
| | (SEQ ID NO: 26) | (SEQ ID NO: 31) |
| 52 | DYKDDDDKDYKDDDDKDYKDDDDK | YPYDVPDYAYPYDVPDYAYPYDVPDYA |
| | (SEQ ID NO: 26) | (SEQ ID NO: 9) |
| 53 | DYKDDDDKDYKDDDDKDYKDDDDK | SWKDASGWSSWKDASGWSSWKDASGWS |
| | (SEQ ID NO: 26) | (SEQ ID NO: 11) |
| 54 | DYKDDDDKDYKDDDDKDYKDDDDK | EQKLISEEDLEQKLISEEDLEQKLISEEDL |
| | (SEQ ID NO: 26) | (SEQ ID NO: 37) |
| 55 | DYKDDDDKDYKDDDDKDYKDDDDK | AWRHPQFGGAWRHPQFGGAWRHPQFGG |
| | (SEQ ID NO: 26) | (SEQ ID NO: 22) |
| 56 | DYKDDDDKDYKDDDDKDYKDDDDK | WSGPQFEKWSGPQFEKWSGPQFEK |
| | (SEQ ID NO: 26) | (SEQ ID NO: 24) |
| 57 | DYKDDDDKDYKDDDDKDYKDDDDK | DLYDHDGDLYDHDIDLYDDDDK |
| | (SEQ ID NO: 26) | (SEQ ID NO: 7) |
| 58 | DYKDHDGDYKDDDDK | DLYDDDDK |
| | (SEQ ID NO: 27) | (SEQ ID NO: 2) |
| 59 | DYKDHDGDYKDDDDK | YPYDVPDYA |
| | (SEQ ID NO: 27) | (SEQ ID NO: 3) |
| 60 | DYKDHDGDYKDDDDK | SWKDASGWS |
| | (SEQ ID NO: 27) | (SEQ ID NO: 4) |
| 61 | DYKDHDGDYKDDDDK | EQKLISEEDL |
| | (SEQ ID NO: 27) | (SEQ ID NO: 5) |
| 62 | DYKDHDGDYKDDDDK | AWRHPQFGG |
| | (SEQ ID NO: 27) | (SEQ ID NO: 19) |
| 63 | DYKDHDGDYKDDDDK | WSGPQFEK |
| | (SEQ ID NO: 27) | (SEQ ID NO: 20) |
| 64 | DYKDHDGDYKDDDDK | DLYDDDDKDLYDDDDK |
| | (SEQ ID NO: 27) | (SEQ ID NO: 30) |
| 65 | DYKDHDGDYKDDDDK | YPYDVPDYAYPYDVPDYA |
| | (SEQ ID NO: 27) | 9SEQ ID NO: 8) |
| 66 | DYKDHDGDYKDDDDK | SWKDASGWSSWKDASGWS |
| | (SEQ ID NO: 27) | (SEQ ID NO: 10) |
| 67 | DYKDHDGDYKDDDDK | EQKLISEEDLEQKLISEEDL |
| | (SEQ ID NO: 27) | (SEQ ID NO: 36) |
| 68 | DYKDHDGDYKDDDDK | AWRHPQFGGAWRHPQFGG |
| | (SEQ ID NO: 27) | (SEQ ID NO: 21) |
| 69 | DYKDHDGDYKDDDDK | WSGPQFEKWSGPQFEK |
| | (SEQ ID NO: 27) | (SEQ ID NO: 23) |
| 70 | DYKDHDGDYKDDDDK | DLYDDDDKDLYDDDDKDLYDDDDK |
| | (SEQ ID NO: 27) | (SEQ ID NO: 31) |
| 71 | DYKDHDGDYKDDDDK | YPYDVPDYAYPYDVPDYAYPYDVPDYA |
| | (SEQ ID NO: 27) | (SEQ ID NO: 9) |
| 72 | DYKDHDGDYKDDDDK | SWKDASGWSSWKDASGWSSWKDASGWS |
| | (SEQ ID NO: 27) | (SEQ ID NO: 11) |
| 73 | DYKDHDGDYKDDDDK | EQKLISEEDLEQKLISEEDLEQKLISEEDL |
| | (SEQ ID NO: 27) | (SEQ ID NO: 37) |
| 74 | DYKDHDGDYKDDDDK | AWRHPQFGGAWRHPQFGGAWRHPQFGG |
| | (SEQ ID NO: 27) | (SEQ ID NO: 22) |
| 75 | DYKDHDGDYKDDDDK | WSGPQFEKWSGPQFEKWSGPQFEK |
| | (SEQ ID NO: 27) | (SEQ ID NO: 24) |
| 76 | DYKDHDGDYKDDDDK | DLYDHDGDLYDHDIDLYDDDDK |
| | (SEQ ID NO: 27) | (SEQ ID NO: 7) |
| 77 | DYKDHDGDYKDHDIDYKDDDDK | DLYDDDD |
| | (SEQ ID NO: 6) | (SEQ ID NO: 2)K |
| 78 | DYKDHDGDYKDHDIDYKDDDDK | YPYDVPDYA |
| | (SEQ ID NO: 6) | (SEQ ID NO: 3) |
| 79 | DYKDHDGDYKDHDIDYKDDDDK | SWKDASGWS |
| | (SEQ ID NO: 6) | (SEQ ID NO: 4) |
| 80 | DYKDHDGDYKDHDIDYKDDDDK | EQKLISEEDL |
| | (SEQ ID NO: 6) | (SQ ID NO: 5) |
| 81 | DYKDHDGDYKDHDIDYKDDDDK | AWRHPQFGG |
| | (SEQ ID NO: 6) | (SEQ ID NO 19) |
| 82 | DYKDHDGDYKDHDIDYKDDDDK | WSGPQFEK |
| | (SEQ ID NO: 6) | (SEQ ID NO: 20) |
| 83 | DYKDHDGDYKDHDIDYKDDDDK | DLYDDDDKDLYDDDDK |
| | (SEQ ID NO: 6) | 9SEQ ID NO: 30) |
| 84 | DYKDHDGDYKDHDIDYKDDDDK | YPYDVPDYAYPYDVPDYA |
| | (SEQ ID NO: 6) | 9SEQ ID NO: 8) |
| 85 | DYKDHDGDYKDHDIDYKDDDDK | SWKDASGWSSWKDASGWS |
| | (SEQ ID NO: 6) | (SEQ ID NO: 10) |
| 86 | DYKDHDGDYKDHDIDYKDDDDK | EQKLISEEDLEQKLISEEDL |
| | (SEQ ID NO: 6) | (SEQ ID NO: 36) |
| 87 | DYKDHDGDYKDHDIDYKDDDDK | AWRHPQFGGAWRHPQFGG |
| | (SEQ ID NO: 6) | (SEQ ID NO: 21) |
| 88 | DYKDHDGDYKDHDIDYKDDDDK | WSGPQFEKWSGPQFEK |
| | (SEQ ID NO: 6) | (SEQ ID NO: 23) |
| 89 | DYKDHDGDYKDHDIDYKDDDDK | DLYDDDDKDLYDDDDKDLYDDDDK |
| | (SEQ ID NO: 6) | (SEQ ID NO: 31) |
| 90 | DYKDHDGDYKDHDIDYKDDDDK | YPYDVPDYAYPYDVPDYAYPYDVPDYA |
| | (SEQ ID NO: 6) | (SEQ ID NO: 9) |
| 91 | DYKDHDGDYKDHDIDYKDDDDK | SWKDASGWSSWKDASGWSSWKDASGWS |
| | (SEQ ID NO: 6) | (SEQ ID NO: 11) |
| 92 | DYKDHDGDYKDHDIDYKDDDDK | EQKLISEEDLEQKLISEEDLEQKLISEEDL |
| | (SEQ ID NO: 6) | (SEQ ID NO: 37) |
| 93 | DYKDHDGDYKDHDIDYKDDDDK | AWRHPQFGGAWRHPQFGGAWRHPQFGG |
| | (SEQ ID NO: 6) | (SEQ ID NO: 22) |
| 94 | DYKDHDGDYKDHDIDYKDDDDK | WSGPQFEKWSGPQFEKWSGPQFEK |
| | (SEQ ID NO: 6) | (SEQ ID NO: 24) |
| 95 | DYKDHDGDYKDHDIDYKDDDDK | DLYDHDGDLYDHDIDLYDDDDK |
| | (SEQ ID NO: 6) | (SEQ ID NO: 7) |
| 96 | DLYDDDDK | DYKDDDDK |
| | (SEQ ID NO: 2) | (SEQ ID NO: 1) |
| 97 | YPYDVPDYA | DYKDDDDK |
| | (SEQ ID NO: 3 | (SEQ ID NO: 1) |
| 98 | SWKDASGWS | DYKDDDDK |
| | (SEQ ID NO: 4) | (SEQ ID NO: 1) |
| 99 | EQKLISEEDL | DYKDDDDK |
| | (SEQ ID NO: 5) | (SEQ ID NO: 1) |
| 100 | AWRHPQFGG | DYKDDDDK |
| | (SEQ ID NO: 19) | (SEQ ID NO: 1) |
| 101 | WSGPQFEK | DYKDDDDK |
| | (SEQ ID NO: 20) | (SEQ ID NO: 1) |
| 102 | DLYDDDDKDLYDDDDK | DYKDDDDK |
| | (SEQ ID NO: 30) | (SEQ ID NO: 1) |
| 103 | YPYDVPDYAYPYDVPDYA | DYKDDDDK |
| | (SEQ ID NO: 8) | (SEQ ID NO: 1) |
| 104 | SWKDASGWSSWKDASGWS | DYKDDDDK |
| | (SEQ ID NO: 10) | (SEQ ID NO: 1) |
| 105 | EQKLISEEDLEQKLISEEDL | DYKDDDDK |
| | (SEQ ID NO: 36) | (SEQ ID NO: 1) |
| 106 | AWRHPQFGGAWRHPQFGG | DYKDDDDK |
| | (SEQ ID NO: 21) | (SEQ ID NO: 1) |
| 107 | WSGPQFEKWSGPQFEK | DYKDDDDK |
| | (SEQ ID NO: 23) | (SEQ ID NO: 1) |
| 108 | DLYDDDDKDLYDDDDKDLYDDDDK | DYKDDDDK |
| | (SEQ ID NO: 31) | (SEQ ID NO: 1) |
| 109 | YPYDVPDYAYPYDVPDYAYPYDVPDYA | DYKDDDDK |
| | (SEQ ID NO: 9) | (SEQ ID NO: 1) |
| 110 | SWKDASGWSSWKDASGWSSWKDASGWS | DYKDDDDK |
| | (SEQ ID NO: 11) | (SEQ ID NO: 1) |
| 111 | QKLISEEDLEQKLISEEDLEQKLISEEDL | DYKDDDDK |
| | (SEQ ID NO: 37) | (SEQ ID NO: 1) |
| 112 | AWRHPQFGGAWRHPQFGGAWRHPQFGG | DYKDDDDK |
| | (SEQ ID NO: 22) | (SEQ ID NO: 1) |
| 113 | WSGPQFEKWSGPQFEKWSGPQFEK | DYKDDDDK |
| | (SEQ ID NO: 24) | (SEQ ID NO: 1) |
| 114 | DLYDHDGDLYDHDIDLYDDDDK | DYKDDDDK |
| | (SEQ ID NO: 7) | (SEQ ID NO: 1) |
| 115 | DLYDDDDK | DYKDDDDKDYKDDDDK |
| | (SEQ ID NO: 2) | (SEQ ID NO: 25) |
| 116 | YPYDVPDYA | DYKDDDDKDYKDDDDK |
| | (SEQ ID NO: 3) | (SEQ ID NO: 25) |
| 117 | SWKDASGWS | DYKDDDDKDYKDDDDK |
| | (SEQ ID NO: 4) | (SEQ ID NO: 25) |
| 118 | EQKLISEEDL | DYKDDDDKDYKDDDDK |
| | (SEQ ID NO: 5) | (SEQ ID NO: 25) |
| 119 | AWRHPQFGG | DYKDDDDKDYKDDDDK |
| | (SEQ ID NO 19) | (SEQ ID NO: 25) |
| 120 | WSGPQFEK | DYKDDDDKDYKDDDDK |
| | (SEQ ID NO: 20) | (SEQ ID NO: 25) |
| 121 | DLYDDDDKDLYDDDDK | DYKDDDDKDYKDDDDK |
| | (SEQ ID NO: 30) | (SEQ ID NO: 25) |
| 122 | YPYDVPDYAYPYDVPDYA | DYKDDDDKDYKDDDDK |
| | (SEQ ID NO: 8) | (SEQ ID NO: 25) |
| 123 | SWKDASGWSSWKDASGWS | DYKDDDDKDYKDDDDK |
| | (SEQ ID NO: 10) | (SEQ ID NO: 25) |
| 124 | EQKLISEEDLEQKLISEEDL | DYKDDDDKDYKDDDDK |
| | (SEQ ID NO: 36) | (SEQ ID NO: 25) |
| 125 | AWRHPQFGGAWRHPQFGG | DYKDDDDKDYKDDDDK |
| | (SEQ ID NO: 21) | (SEQ ID NO: 25) |
| 126 | WSGPQFEKWSGPQFEK | DYKDDDDKDYKDDDDK |
| | (SEQ ID NO: 23) | (SEQ ID NO: 25) |
| 127 | DLYDDDDKDLYDDDDKDLYDDDDK | DYKDDDDKDYKDDDDK |
| | (SEQ ID NO: 31) | (SEQ ID NO: 25) |
| 128 | YPYDVPDYAYPYDVPDYAYPYDVPDYA | DYKDDDDKDYKDDDDK |
| | (SEQ ID NO: 9) | (SEQ ID NO: 25) |
| 129 | SWKDASGWSSWKDASGWSSWKDASGWS | DYKDDDDKDYKDDDDK |
| | (SEQ ID NO: 11) | (SEQ ID NO: 25) |
| 130 | EQKLISEEDLEQKLISEEDLEQKLISEEDL | DYKDDDDKDYKDDDDK |
| | (SEQ ID NO: 37) | (SEQ ID NO: 25) |
| 131 | AWRHPQFGGAWRHPQFGGAWRHPQFGG | DYKDDDDKDYKDDDDK |
| | (SEQ ID NO: 22) | (SEQ ID NO: 25) |
| 132 | WSGPQFEKWSGPQFEKWSGPQFEK | DYKDDDDKDYKDDDDK |
| | (SEQ ID NO: 24) | (SEQ ID NO: 25) |
| 133 | DLYDHDGDLYDHDIDLYDDDDK | DYKDDDDKDYKDDDDK |
| | (SEQ ID NO: 7) | (SEQ ID NO: 25) |
| 134 | DLYDDDDK | DYKDDDDKDYKDDDDKDYKDDDDK |
| | (SEQ ID NO: 2) | (SEQ ID NO: 26) |
| 135 | YPYDVPDYA | DYKDDDDKDYKDDDDKDYKDDDDK |
| | (SEQ ID NO: 3) | (SEQ ID NO: 26) |
| 136 | SWKDASGWS | DYKDDDDKDYKDDDDKDYKDDDDK |
| | (SEQ ID NO: 4) | (SEQ ID NO: 26) |
| 137 | EQKLISEEDL | DYKDDDDKDYKDDDDKDYKDDDDK |
| | (SEQ ID NO: 5) | (SEQ ID NO: 26) |
| 138 | AWRHPQFGG | DYKDDDDKDYKDDDDKDYKDDDDK |
| | (SEQ ID NO: 19) | (SEQ ID NO: 26) |
| 139 | WSGPQFEK | DYKDDDDKDYKDDDDKDYKDDDDK |
| | (SEQ ID NO: 20) | (SEQ ID NO: 26) |
| 140 | DLYDDDDKDLYDDDDK | DYKDDDDKDYKDDDDKDYKDDDDK |
| | (SEQ ID NO: 30) | (SEQ ID NO: 26) |
| 141 | YPYDVPDYAYPYDVPDYA | DYKDDDDKDYKDDDDKDYKDDDDK |
| | (SEQ ID NO: 8) | (SEQ ID NO: 26) |
| 142 | SWKDASGWSSWKDASGWS | DYKDDDDKDYKDDDDKDYKDDDDK |
| | (SEQ ID NO: 10) | (SEQ ID NO: 26) |
| 143 | EQKLISEEDLEQKLISEEDL | DYKDDDDKDYKDDDDKDYKDDDDK |
| | (SEQ ID NO: 36) | (SEQ ID NO: 26) |
| 144 | AWRHPQFGGAWRHPQFGG | DYKDDDDKDYKDDDDKDYKDDDDK |
| | (SEQ ID NO: 21) | (SEQ ID NO: 26) |
| 145 | WSGPQFEKWSGPQFEK | DYKDDDDKDYKDDDDKDYKDDDDK |
| | (SEQ ID NO: 23) | (SEQ ID NO: 26) |
| 146 | DLYDDDDKDLYDDDDKDLYDDDD | DYKDDDDKDYKDDDDKDYKDDDDK |
| | (SEQ ID NO: 31) | (SEQ ID NO: 26) |
| 147 | YPYDVPDYAYPYDVPDYAYPYDVPDYA | DYKDDDDKDYKDDDDKDYKDDDDK |
| | (SEQ ID NO: 9) | (SEQ ID NO: 26) |
| 148 | SWKDASGWSSWKDASGWSSWKDASGWS | DYKDDDDKDYKDDDDKDYKDDDDK |
| | (SEQ ID NO: 11) | (SEQ ID NO: 26) |
| 149 | EQKLISEEDLEQKLISEEDLEQKLISEEDL | DYKDDDDKDYKDDDDKDYKDDDDK |
| | (SEQ ID NO: 37) | (SEQ ID NO: 26) |
| 150 | AWRHPQFGGAWRHPQFGGAWRHPQFGG | DYKDDDDKDYKDDDDKDYKDDDDK |
| | (SEQ ID NO: 22) | (SEQ ID NO: 26) |
| 151 | WSGPQFEKWSGPQFEKWSGPQF | DYKDDDDKDYKDDDDKDYKDDDDK |
| | (SEQ ID NO: 24) | (SEQ ID NO: 26) |
| 152 | DLYDHDGDLYDHDIDLYDDDDK | DYKDDDDKDYKDDDDKDYKDDDDK |
| | (SEQ ID NO: 7) | (SEQ ID NO: 26) |
| 153 | DLYDDDDK | DYKDHDGDYKDDDDK |
| | (SEQ ID NO: 2) | (SEQ ID NO: 27) |
| 154 | YPYDVPDYA | DYKDHDGDYKDDDDK |
| | (SEQ ID NO: 3) | (SEQ ID NO: 27) |
| 155 | SWKDASGWS | DYKDHDGDYKDDDDK |
| | (SEQ ID NO: 4) | (SEQ ID NO: 27) |
| 156 | EQKLISEEDL | DYKDHDGDYKDDDDK |
| | (SEQ ID NO: 5) | (SEQ ID NO: 27) |
| 157 | AWRHPQFGG | DYKDHDGDYKDDDDK |
| | (SEQ ID NO: 19) | (SEQ ID NO: 27) |
| 158 | WSGPQFEK | DYKDHDGDYKDDDDK |
| | (SEQ ID NO: 20) | (SEQ ID NO: 27) |
| 159 | DLYDDDDKDLYDDDDK | DYKDHDGDYKDDDDK |
| | (SEQ ID NO: 30) | (SEQ ID NO: 27) |
| 160 | YPYDVPDYAYPYDVPDYA | DYKDHDGDYKDDDDK |
| | (SEQ ID NO: 8) | (SEQ ID NO: 27) |
| 161 | SWKDASGWSSWKDASGWS | DYKDHDGDYKDDDDK |
| | (SEQ ID NO: 10) | (SEQ ID NO: 27) |
| 162 | EQKLISEEDLEQKLISEEDL | DYKDHDGDYKDDDDK |
| | (SEQ ID NO: 36) | (SEQ ID NO: 27) |
| 163 | AWRHPQFGGAWRHPQFGG | DYKDHDGDYKDDDDK |
| | (SEQ ID NO: 21) | (SEQ ID NO: 27) |
| 164 | WSGPQFEKWSGPQFEK | DYKDHDGDYKDDDDK |
| | (SEQ ID NO: 23) | (SEQ ID NO: 27) |
| 165 | DLYDDDDKDLYDDDDKDLYDDDDK | DYKDHDGDYKDDDDK |
| | (SEQ ID NO: 31) | (SEQ ID NO: 27) |
| 166 | YPYDVPDYAYPYDVPDYAYPYDVPDYA | DYKDHDGDYKDDDDK |
| | (SEQ ID NO: 9) | (SEQ ID NO: 27) |
| 167 | SWKDASGWSSWKDASGWSSWKDASGWS | DYKDHDGDYKDDDDK |
| | (SEQ ID NO: 11) | (SEQ ID NO: 27) |
| 168 | EQKLISEEDLEQKLISEEDLEQKLISEEDL | DYKDHDGDYKDDDDK |
| | (SEQ ID NO: 37) | (SEQ ID NO: 27) |
| 169 | AWRHPQFGGAWRHPQFGGAWR | DYKDHDGDYKDDDDK |
| | (SEQ ID NO: 22) | (SEQ ID NO: 27) |
| 170 | WSGPQFEKWSGPQFEKWSGPQFEK | DYKDHDGDYKDDDDK |
| | (SEQ ID NO: 24) | (SEQ ID NO: 27) |
| 171 | DLYDHDGDLYDHDIDLYDDDDK | DYKDHDGDYKDDDDK |
| | (SEQ ID NO: 7) | (SEQ ID NO: 27) |
| 172 | DLYDDDDK | DYKDHDGDYKDHDIDYKDDDDK |
| | (SEQ ID NO: 2) | (SEQ ID NO: 6) |
| 173 | YPYDVPDYA | DYKDHDGDYKDHDIDYKDDDDK |
| | (SEQ ID NO: 3) | (SEQ ID NO: 6) |
| 174 | SWKDASGWS | DYKDHDGDYKDHDIDYKDDDDK |
| | (SEQ ID NO: 4) | (SEQ ID NO: 6) |
| 175 | EQKLISEEDL | DYKDHDGDYKDHDIDYKDDDDK |
| | (SEQ ID NO: 5) | (SEQ ID NO: 6) |
| 176 | AWRHPQFGG | DYKDHDGDYKDHDIDYKDDDDK |
| | (SEQ ID NO: 19) | (SEQ ID NO: 6) |
| 177 | WSGPQFEK | DYKDHDGDYKDHDIDYKDDDDK |
| | (SEQ ID NO: 20) | (SEQ ID NO: 6) |
| 178 | DLYDDDDKDLYDDDDK | DYKDHDGDYKDHDIDYKDDDDK |
| | (SEQ ID NO: 30) | (SEQ ID NO: 6) |
| 179 | YPYDVPDYAYPYDVPDYA | DYKDHDGDYKDHDIDYKDDDDK |
| | (SEQ ID NO: 8) | (SEQ ID NO: 6) |
| 180 | SWKDASGWSSWKDASGWS | DYKDHDGDYKDHDIDYKDDDDK |
| | (SEQ ID NO: 10) | (SEQ ID NO: 6) |
| 181 | EQKLISEEDLEQKLISEEDL | DYKDHDGDYKDHDIDYKDDDDK |
| | (SEQ ID NO: 36) | (SEQ ID NO: 6) |
| 182 | AWRHPQFGGAWRHPQFGG | DYKDHDGDYKDHDIDYKDDDDK |
| | (SEQ ID NO: 21) | (SEQ ID NO: 6) |
| 183 | WSGPQFEKWSGPQFEK | DYKDHDGDYKDHDIDYKDDDDK |
| | (SEQ ID NO: 23) | (SEQ ID NO: 6) |
| 184 | DLYDDDDKDLYDDDDKDLYDDDDK | DYKDHDGDYKDHDIDYKDDDDK |
| | (SEQ ID NO: 31) | (SEQ ID NO: 6) |
| 185 | YPYDVPDYAYPYDVPDYAYPYDVPDYA | DYKDHDGDYKDHDIDYKDDDDK |
| | (SEQ ID NO: 9) | (SEQ ID NO: 6) |
| 186 | SWKDASGWSSWKDASGWSSWKDASGWS | DYKDHDGDYKDHDIDYKDDDDK |
| | (SEQ ID NO: 11) | (SEQ ID NO: 6) |
| 187 | EQKLISEEDLEQKLISEEDLEQKLISEEDL | DYKDHDGDYKDHDIDYKDDDDK |
| | (SEQ ID NO: 37) | (SEQ ID NO: 6) |
| 188 | AWRHPQFGGAWRHPQFGGAWRHPQFGG | DYKDHDGDYKDHDIDYKDDDDK |
| | (SEQ ID NO: 22) | (SEQ ID NO: 6) |
| 189 | WSGPQFEKWSGPQFEKWSGPQFEK | DYKDHDGDYKDHDIDYKDDDDK |
| | (SEQ ID NO: 24) | (SEQ ID NO: 6) |
| 190 | DLYDHDGDLYDHDIDLYDDDDK | DYKDHDGDYKDHDIDYKDDDDK |
| | (SEQ ID NO: 7) | (SEQ ID NO: 6) |

24. A kit for isolating the nucleic acid in claim 1 from a sample, the kit comprising:
(a.) a plurality of agents for isolating, capturing and purifying the nucleic acid; and
(b.) instructions for isolating, capturing and purifying the nucleic acid.


SEQUENCE LISTING (49 sequences)

SEQ ID NO: 1 - protein, 8 aa, Artificial Sequence
  Description of Artificial Sequence Synthetic peptide
DYKDDDDK

SEQ ID NO: 2 - protein, 8 aa, Artificial Sequence
  Description of Artificial Sequence Synthetic peptide
DLYDDDDK

SEQ ID NO: 3 - protein, 9 aa, Artificial Sequence
  Description of Artificial Sequence Synthetic peptide
YPYDVPDYA

SEQ ID NO: 4 - protein, 9 aa, Artificial Sequence
  Description of Artificial Sequence Synthetic peptide
SWKDASGWS

SEQ ID NO: 5 - protein, 10 aa, Artificial Sequence
  Description of Artificial Sequence Synthetic peptide
EQKLISEEDL

SEQ ID NO: 6 - protein, 22 aa, Artificial Sequence
  Description of Artificial Sequence Synthetic peptide
DYKDHDGDYKDHDIDYKDDDDK

SEQ ID NO: 7 - protein, 22 aa, Artificial Sequence
  Description of Artificial Sequence Synthetic peptide
DLYDHDGDLYDHDIDLYDDDDK

SEQ ID NO: 8 - protein, 18 aa, Artificial Sequence
  Description of Artificial Sequence Synthetic peptide
YPYDVPDYAYPYDVPDYA

SEQ ID NO: 9 - protein, 27 aa, Artificial Sequence
  Description of Artificial Sequence Synthetic peptide
YPYDVPDYAYPYDVPDYAYPYDVPDYA

SEQ ID NO: 10 - protein, 18 aa, Artificial Sequence
  Description of Artificial Sequence Synthetic peptide
SWKDASGWSSWKDASGWS

SEQ ID NO: 11 - protein, 27 aa, Artificial Sequence
  Description of Artificial Sequence Synthetic peptide
SWKDASGWSSWKDASGWSSWKDASGWS

SEQ ID NO: 12 - protein, 5 aa, Artificial Sequence
  Description of Artificial Sequence Synthetic peptide
DDDDK

SEQ ID NO: 13 - protein, 23 aa, Artificial Sequence
  Description of Artificial Sequence Synthetic peptide
MDYKDHDGDYKDHDIDYKDDDDK

SEQ ID NO: 14 - protein, 23 aa, Artificial Sequence
  Description of Artificial Sequence Synthetic peptide
MDLYDHDGDLYDHDIDLYDDDDK

SEQ ID NO: 15 - protein, 6 aa, Artificial Sequence
  Description of Artificial Sequence Synthetic peptide
  MOD_RES (6)..(6): non-acidic amino acid
LVPRGX

SEQ ID NO: 16 - protein, 5 aa, Artificial Sequence
  Description of Artificial Sequence Synthetic peptide
  MOD_RES (5)..(5): variable amino acid other than Pro or Arg
IEGRX

SEQ ID NO: 17 - protein, 5 aa, Artificial Sequence
  Description of Artificial Sequence Synthetic peptide
  MOD_RES (5)..(5): variable amino acid other than Pro or Arg
IDGRX

SEQ ID NO: 18 - protein, 5 aa, Artificial Sequence
  Description of Artificial Sequence Synthetic peptide
  MOD_RES (5)..(5): variable amino acid other than Pro or Arg
AEGRX

SEQ ID NO: 19 - protein, 9 aa, Artificial Sequence
  Description of Artificial Sequence Synthetic peptide
AWRHPQFGG

SEQ ID NO: 20 - protein, 8 aa, Artificial Sequence
  Description of Artificial Sequence Synthetic peptide
WSGPQFEK

SEQ ID NO: 21 - protein, 18 aa, Artificial Sequence
  Description of Artificial Sequence Synthetic peptide
AWRHPQFGGAWRHPQFGG

SEQ ID NO: 22 - protein, 27 aa, Artificial Sequence
  Description of Artificial Sequence Synthetic peptide
AWRHPQFGGAWRHPQFGGAWRHPQFGG

SEQ ID NO: 23 - protein, 16 aa, Artificial Sequence
  Description of Artificial Sequence Synthetic peptide
WSGPQFEKWSGPQFEK

SEQ ID NO: 24 - protein, 24 aa, Artificial Sequence
  Description of Artificial Sequence Synthetic peptide
WSGPQFEKWSGPQFEKWSGPQFEK

SEQ ID NO: 25 - protein, 16 aa, Artificial Sequence
  Description of Artificial Sequence Synthetic peptide
DYKDDDDKDYKDDDDK

SEQ ID NO: 26 - protein, 24 aa, Artificial Sequence
  Description of Artificial Sequence Synthetic peptide
DYKDDDDKDYKDDDDKDYKDDDDK

SEQ ID NO: 27 - protein, 15 aa, Artificial Sequence
  Description of Artificial Sequence Synthetic peptide
DYKDHDGDYKDDDDK

SEQ ID NO: 28 - protein, 15 aa, Artificial Sequence
  Description of Artificial Sequence Synthetic peptide
DYDDHDIDYKDDDDK

SEQ ID NO: 29 - protein, 707 aa, Simian virus 40
MDKVLNREESLQLMDLLGLERSAWGNIPLMRKAYLKKCKEFHPDKGGDEEKMKKMNTLYKKMEDGVKYAHQPDFGGFWDATEIPTYGTDEWEQWWNAFNEENLFCSEEMPSSDDEATADSQHSTPPKKKRKVEDPKDFPSELLSFLSHAVFSNRTLACFAIYTTKEKAALLYKKIMEKYSVTFISRHNSYNHNILFFLTPHRHRVSAINNYAQKLCTFSFLICKGVNKEYLMYSALTRDPFSVIEESLPGGLKEHDFNPEEAEETKQVSWKLVTEYAMETKCDDVLLLLGMYLEFQYSFEMCLKCIKKEQPSHYKYHEKHYANAAIFADSKNQKTICQQAVDTVLAKKRVDSLQLTREQMLTNRFNDLLDRMDIMFGSTGSADIEEWMAGVAWLHCLLPKMDSVVYDFLKCMVYNIPKKRYWLFKGPIDSGKTTLAAALLELCGGKALNVNLPLDRLNFELGVAIDQFLVVFEDVKGTGGESRDLPSGQGINNLDNLRDYLDGSVKVNLEKKHLNKRTQIFPPGIVTMNEYSVPKTLQARFVKQIDFRPKDYLKHCLERSEFLLEKRIIQSGIALLLMLIWYRPVAEFAQSIQSRIVEWKERLDKEFSLSVYQKMKFNVAMGVGVLDWLRNSDDDDDDSQENADKNEDKNMEDSGHETGIDSQSQGSFQAQPSQSSQSVHDHNQPYHICRGFTCFKKPPTPPPEPET

SEQ ID NO: 30 - protein, 16 aa, Artificial Sequence
  Description of Artificial Sequence Synthetic peptide
DLYDDDDKDLYDDDDK

SEQ ID NO: 31 - protein, 24 aa, Artificial Sequence
  Description of Artificial Sequence Synthetic peptide
DLYDDDDKDLYDDDDKDLYDDDDK

SEQ ID NO: 32 - protein, 15 aa, Artificial Sequence
  Description of Artificial Sequence Synthetic peptide
DLYDHDGDLYDDDDK

SEQ ID NO: 33 - protein, 15 aa, Artificial Sequence
  Description of Artificial Sequence Synthetic peptide
DLYDHDIDLYDDDDK

SEQ ID NO: 34 - protein, 4 aa, Artificial Sequence
  Description of Artificial Sequence Synthetic peptide
DDDK

SEQ ID NO: 35 - protein, 7 aa, Artificial Sequence
  Description of Artificial Sequence Synthetic peptide
  MOD_RES (2)..(2): Ala, Arg, Asn, Asp, Gln, Glu, Ile, Lys, Phe, Pro, Ser,Thr, Trp or Val
  MOD_RES (6)..(6): Ala, Arg, Asn, Asp, Cys, Gln, Glu, Gly, Ile, Leu, Lys,Met, Pro, Ser, Thr, Tyr or Val
HXHRHXH

SEQ ID NO: 36 - protein, 20 aa, Artificial Sequence
  Description of Artificial Sequence Synthetic peptide
EQKLISEEDLEQKLISEEDL

SEQ ID NO: 37 - protein, 30 aa, Artificial Sequence
  Description of Artificial Sequence Synthetic peptide
EQKLISEEDLEQKLISEEDLEQKLISEEDL

SEQ ID NO: 38 - protein, 23 aa, Artificial Sequence
  Description of Artificial Sequence Synthetic peptide
  MOD_RES (1)..(1): variable amino acid or not present
  MOD_RES (4)..(5): variable amino acid
  MOD_RES (7)..(7): variable amino acid or not present
  misc_feature (8)..(14): region may or may not be present
  MOD_RES (8)..(8): variable amino acid or not present
  MOD_RES (11)..(12): variable amino acid
  MOD_RES (14)..(14): variable amino acid or not present
  MOD_RES (15)..(15): variable amino acid
  MOD_RES (16)..(16): variable amino acid or not present
  MOD_RES (19)..(20): variable amino acid
  MOD_RES (22)..(22): may or may not be present
  see specification as filed for detailed description of substitutions and preferred embodiments
XYKXXDXXYKXXDXXXYKXXDDK

SEQ ID NO: 39 - protein, 21 aa, Artificial Sequence
  Description of Artificial Sequence Synthetic peptide
  MOD_RES (4)..(5): Variable amino acid
  misc_feature (7)..(12): region may or may not be present
  MOD_RES (10)..(11): Variable amino acid
  MOD_RES (13)..(13): Variable amino acid
  MOD_RES (17)..(18): Variable amino acid
  MOD_RES (20)..(20): Variable amino acid
  see specification as filed for detailed description of substitutions and preferred embodiments
DYKXXDDYKXXDXDYKXXDXK

SEQ ID NO: 40 - protein, 12 aa, Artificial Sequence
  Description of Artificial Sequence Synthetic peptide
  MOD_RES (4)..(5): Variable amino acid
  MOD_RES (10)..(11): Variable amino acid
  see specification as filed for detailed description of substitutions and preferred embodiments
DYKXXDDYKXXD

SEQ ID NO: 41 - protein, 6 aa, Artificial Sequence
  Description of Artificial Sequence Synthetic peptide
  MOD_RES (4)..(5): Variable amino acid
  see specification as filed for detailed description of substitutions and preferred embodiments
DYKXXD

SEQ ID NO: 42 - protein, 8 aa, Artificial Sequence
  Description of Artificial Sequence Synthetic peptide
  MOD_RES (4)..(5): Variable amino acid
  MOD_RES (7)..(7): may or may not be present
  see specification as filed for detailed description of substitutions and preferred embodiments
DYKXXDDK

SEQ ID NO: 43 - protein, 22 aa, Artificial Sequence
  Description of Artificial Sequence Synthetic peptide
  MOD_RES (1)..(1): Variable amino acid or not present; if present preferably Met
  MOD_RES (5)..(6): Variable amino acid
  MOD_RES (11)..(12): Variable amino acid
  MOD_RES (14)..(14): Variable amino acid or not present
  MOD_RES (18)..(19): Variable amino acid
  MOD_RES (21)..(21): may or may not be present
  see specification as filed for detailed description of substitutions and preferred embodiments
XDYKXXDDYKXXDXDYKXXDDK

SEQ ID NO: 44 - protein, 14 aa, Artificial Sequence
  Description of Artificial Sequence Synthetic peptide
  misc_feature (1)..(5): region may or may not be present
  MOD_RES (2)..(2): Variable amino acid or not present; if present preferably Leu
  MOD_RES (4)..(4): Variable amino acid
  MOD_RES (5)..(5): Variable amino acid or not present
  MOD_RES (6)..(6): Variable amino acid or not present
  MOD_RES (8)..(8): Variable amino acid or not present; if present preferably Leu
  MOD_RES (10)..(10): Variable amino acid
  MOD_RES (11)..(11): Variable amino acid or not present
  MOD_RES (13)..(13): may or may not be present
  see specification as filed for detailed description of substitutions and preferred embodiments
DXYXXXDXYXXDDK

SEQ ID NO: 45 - protein, 8 aa, Artificial Sequence
  Description of Artificial Sequence Synthetic peptide
  MOD_RES (2)..(2): Variable amino acid or not present; if present preferably Leu
  MOD_RES (4)..(4): Variable amino acid or not present; if present preferably Asp
  MOD_RES (5)..(5): Variable amino acid or not present; if present preferably Asp
  MOD_RES (7)..(7): may or may not be present
  see specification as filed for detailed description of substitutions and preferred embodiments
DXYXXDDK

SEQ ID NO: 46 - protein, 15 aa, Artificial Sequence
  Description of Artificial Sequence Synthetic peptide
  MOD_RES (1)..(1): Variable amino acid or not present; if present preferably Met
  misc_feature (2)..(6): region may or may not be present
  MOD_RES (3)..(3): Variable amino acid or not present; if present preferably Leu
  MOD_RES (5)..(5): Variable amino acid
  MOD_RES (6)..(6): Variable amino acid or not present
  MOD_RES (7)..(7): Variable amino acid or not present
  MOD_RES (9)..(9): Variable amino acid or not present; if present preferably Leu
  MOD_RES (11)..(11): Variable amino acid
  MOD_RES (12)..(12): Variable amino acid or not present
  MOD_RES (14)..(14): may or may not be present
  see specification as filed for detailed description of substitutions and preferred embodiments
XDXYXXXDXYXXDDK

SEQ ID NO: 47 - DNA, 73 bp, Artificial Sequence
  Description of Artificial Sequence Synthetic oligonucleotide
atggactacaaggacgacgatgacaaagtcaagtctacccatacgatgttccagattacgctgctgctatgcc

SEQ ID NO: 48 - protein, 22 aa, Artificial Sequence
  Description of Artificial Sequence Synthetic peptide
DYKDDDDKVKLYPYDVPDYAAA

SEQ ID NO: 49 - protein, 8 aa, Artificial Sequence
  Description of Artificial Sequence Synthetic peptide
  MOD_RES (1)..(1): Variable amino acid or not present
  MOD_RES (4)..(4): Variable amino acid
  MOD_RES (5)..(5): Variable amino acid
  MOD_RES (7)..(7): may or may not be present
  see specification as filed for detailed description of substitutions and preferred embodiments
XYKXXDDK